SEQUENCE LISTING (10 sequences)

SEQ ID NO: 1 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 2 - protein, 153 aa, Homo sapiens
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 3 - DNA, 531 bp, Homo sapiens
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgag

SEQ ID NO: 4 - DNA, 534 bp, Homo sapiens
atggaaatctgcagaggcctgcggagccacctgattaccctgctgctgttcctgttccacagcgagacaatctgccggcccagcggccggaagtccagcaagatgcaggccttccggatctgggacgtgaaccagaaaaccttctacctgcggaacaaccagctggtggccggatacctgcagggccccaacgtgaacctggaagagaagatcgacgtggtgcccatcgagccccacgccctgtttctgggcatccacggcggcaagatgtgcctgagctgcgtgaagtccggcgacgagacaagactgcagctggaagccgtgaacatcaccgacctgagcgagaaccggaagcaggacaagagattcgccttcatcagaagcgacagcggccccaccaccagctttgagagcgccgcctgccccggctggttcctgtgtacagccatggaagccgaccagcccgtgtccctgacaaacatgcccgacgagggcgtgatggtcaccaagttctattttcaagaagatgagtaa

SEQ ID NO: 5 - DNA, 531 bp, Homo sapiens
atggaaatttgccgcggcctgcgcagccatctgattaccctgctgctgtttctgtttcatagcgaaaccatttgccgcccgagcggccgcaaaagcagcaaaatgcaggcgtttcgcatttgggatgtgaaccagaaaaccttttatctgcgcaacaaccagctggtggcgggctatctgcagggcccgaacgtgaacctggaagaaaaaattgatgtggtgccgattgaaccgcatgcgctgtttctgggcattcatggcggcaaaatgtgcctgagctgcgtgaaaagcggcgatgaaacccgcctgcagctggaagcggtgaacattaccgatctgagcgaaaaccgcaaacaggataaacgctttgcgtttattcgcagcgatagcggcccgaccaccagctttgaaagcgcggcgtgcccgggctggtttctgtgcaccgcgatggaagcggatcagccggtgagcctgaccaacatgccggatgaaggcgtgatggtgaccaaattttattttcaggaagatgaa

SEQ ID NO: 6 - DNA, 531 bp, Homo sapiens
  misc_feature (15)..(15): n is a, c, g, or t
  misc_feature (18)..(18): n is a, c, g, or t
  misc_feature (21)..(21): n is a, c, g, or t
  misc_feature (24)..(24): n is a, c, g, or t
  misc_feature (27)..(27): n is a, c, g, or t
  misc_feature (33)..(33): n is a, c, g, or t
  misc_feature (39)..(39): n is a, c, g, or t
  misc_feature (42)..(42): n is a, c, g, or t
  misc_feature (45)..(45): n is a, c, g, or t
  misc_feature (48)..(48): n is a, c, g, or t
  misc_feature (54)..(54): n is a, c, g, or t
  misc_feature (63)..(63): n is a, c, g, or t
  misc_feature (69)..(69): n is a, c, g, or t
  misc_feature (78)..(78): n is a, c, g, or t
  misc_feature (81)..(81): n is a, c, g, or t
  misc_feature (84)..(84): n is a, c, g, or t
  misc_feature (87)..(87): n is a, c, g, or t
  misc_feature (90)..(90): n is a, c, g, or t
  misc_feature (96)..(96): n is a, c, g, or t
  misc_feature (99)..(99): n is a, c, g, or t
  misc_feature (111)..(111): n is a, c, g, or t
  misc_feature (117)..(117): n is a, c, g, or t
  misc_feature (129)..(129): n is a, c, g, or t
  misc_feature (141)..(141): n is a, c, g, or t
  misc_feature (150)..(150): n is a, c, g, or t
  misc_feature (153)..(153): n is a, c, g, or t
  misc_feature (165)..(165): n is a, c, g, or t
  misc_feature (168)..(168): n is a, c, g, or t
  misc_feature (171)..(171): n is a, c, g, or t
  misc_feature (174)..(174): n is a, c, g, or t
  misc_feature (180)..(180): n is a, c, g, or t
  misc_feature (186)..(186): n is a, c, g, or t
  misc_feature (189)..(189): n is a, c, g, or t
  misc_feature (195)..(195): n is a, c, g, or t
  misc_feature (201)..(201): n is a, c, g, or t
  misc_feature (219)..(219): n is a, c, g, or t
  misc_feature (222)..(222): n is a, c, g, or t
  misc_feature (225)..(225): n is a, c, g, or t
  misc_feature (234)..(234): n is a, c, g, or t
  misc_feature (240)..(240): n is a, c, g, or t
  misc_feature (243)..(243): n is a, c, g, or t
  misc_feature (249)..(249): n is a, c, g, or t
  misc_feature (252)..(252): n is a, c, g, or t
  misc_feature (261)..(261): n is a, c, g, or t
  misc_feature (264)..(264): n is a, c, g, or t
  misc_feature (276)..(276): n is a, c, g, or t
  misc_feature (279)..(279): n is a, c, g, or t
  misc_feature (285)..(285): n is a, c, g, or t
  misc_feature (291)..(291): n is a, c, g, or t
  misc_feature (294)..(294): n is a, c, g, or t
  misc_feature (303)..(303): n is a, c, g, or t
  misc_feature (306)..(306): n is a, c, g, or t
  misc_feature (309)..(309): n is a, c, g, or t
  misc_feature (315)..(315): n is a, c, g, or t
  misc_feature (321)..(321): n is a, c, g, or t
  misc_feature (324)..(324): n is a, c, g, or t
  misc_feature (333)..(333): n is a, c, g, or t
  misc_feature (339)..(339): n is a, c, g, or t
  misc_feature (342)..(342): n is a, c, g, or t
  misc_feature (351)..(351): n is a, c, g, or t
  misc_feature (366)..(366): n is a, c, g, or t
  misc_feature (372)..(372): n is a, c, g, or t
  misc_feature (381)..(381): n is a, c, g, or t
  misc_feature (384)..(384): n is a, c, g, or t
  misc_feature (390)..(390): n is a, c, g, or t
  misc_feature (393)..(393): n is a, c, g, or t
  misc_feature (396)..(396): n is a, c, g, or t
  misc_feature (399)..(399): n is a, c, g, or t
  misc_feature (402)..(402): n is a, c, g, or t
  misc_feature (405)..(405): n is a, c, g, or t
  misc_feature (414)..(414): n is a, c, g, or t
  misc_feature (417)..(417): n is a, c, g, or t
  misc_feature (420)..(420): n is a, c, g, or t
  misc_feature (426)..(426): n is a, c, g, or t
  misc_feature (429)..(429): n is a, c, g, or t
  misc_feature (438)..(438): n is a, c, g, or t
  misc_feature (444)..(444): n is a, c, g, or t
  misc_feature (447)..(447): n is a, c, g, or t
  misc_feature (456)..(456): n is a, c, g, or t
  misc_feature (465)..(465): n is a, c, g, or t
  misc_feature (468)..(468): n is a, c, g, or t
  misc_feature (471)..(471): n is a, c, g, or t
  misc_feature (474)..(474): n is a, c, g, or t
  misc_feature (477)..(477): n is a, c, g, or t
  misc_feature (486)..(486): n is a, c, g, or t
  misc_feature (495)..(495): n is a, c, g, or t
  misc_feature (498)..(498): n is a, c, g, or t
  misc_feature (504)..(504): n is a, c, g, or t
  misc_feature (507)..(507): n is a, c, g, or t
atggarathtgymgnggnytnmgnwsncayytnathacnytnytnytnttyytnttycaywsngaracnathtgymgnccnwsnggnmgnaarwsnwsnaaratgcargcnttymgnathtgggaygtnaaycaraaracnttytayytnmgnaayaaycarytngtngcnggntayytncarggnccnaaygtnaayytngargaraarathgaygtngtnccnathgarccncaygcnytnttyytnggnathcayggnggnaaratgtgyytnwsntgygtnaarwsnggngaygaracnmgnytncarytngargcngtnaayathacngayytnwsngaraaymgnaarcargayaarmgnttygcnttyathmgnwsngaywsnggnccnacnacnwsnttygarwsngcngcntgyccnggntggttyytntgyacngcnatggargcngaycarccngtnwsnytnacnaayatgccngaygarggngtnatggtnacnaarttytayttycargargaygar

SEQ ID NO: 7 - DNA, 2208 bp, adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - protein, 735 aa, adeno-associated virus [sequence omitted]

SEQ ID NO: 9 - DNA, 2211 bp, adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcgcttcaacgggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacgccaagtctgccaatgtggactttactgtggacaataatggcgtgtattcagagcctcgccccattggcaccagatacctgactcgtaatctgtaa

SEQ ID NO: 10 - protein, 736 aa, adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYAKSANVDFTVDNNGVYSEPRPIGTRYLTRNL

(19) United States
(12) Patent Application Publication
(10) Pub. No.: US 2022/0265773 A1
(43) Pub. Date: Aug. 25, 2022

(54) METHODS AND COMPOSITIONS FOR TREATING ARTHRITIS

(71) Applicant: New York University, New York, NY (US)

(72) Inventors: Steven ABRAMSON, New York, NY (US); Mukundan ATTUR, Woodside, NY (US)

(21) Appl. No.: 17/630,625

(22) PCT Filed: Jul. 31, 2020
(86) PCT No.: PCT/US2020/044610

§ 371 Date: Jan. 27, 2022

Related U.S. Application Data
(60) Provisional application No. 62/882,348, filed on Aug. 2, 2019.

(51) Int. Cl. A61K 38/20; A61P 19/02; C12N 15/86; A61K 9/00

(52) U.S. Cl. CPC A61K 38/2006; A61P 19/02; C12N 2740/15043; A61K 9/0019; C12N 15/86

(57) ABSTRACT

Methods for treating a subject for arthritis are provided. Aspects of the methods include administration to the subject a rs419598/rs315952/rs9005-haplotype-informed therapeutic regimen. In some instances, the methods include administering to the subject a therapeutic regimen that antagonizes interleukin-1 (IL-1) activity and/or a disease modifying osteoarthritis drug (DMOAD) if the subject has been identified as having a TTG rs419598/rs315952/rs9005 haplotype. Also provided are compositions for use in practicing the methods.

### CROSS-REFERENCE TO RELATED APPLICATION

Pursuant to 35 U.S.C. § 119(e), this application claims priority to the filing date of the U.S. Provisional Patent Application Ser. No. 62/882,348 filed Aug. 2, 2019; the disclosure of which application is herein incorporated by reference.

### INTRODUCTION

Arthritis is a term used to refer to the swelling and tenderness of one or more joints. Joint pain and stiffness are the main symptoms of arthritis. These symptoms may worsen in older patients. The most common types of arthritis are osteoarthritis and rheumatoid arthritis.

Osteoarthritis (OA) affects over 27 million Americans and is the leading cause of disability among the elderly (Lawrence, et al., “Estimates of the prevalence of arthritis and other rheumatic conditions in the United States. Part II.” Arthritis and rheumatism (2008) 58(1):26-35). Patients with OA are also at higher risk of death (Nuesch et al., “All cause and disease specific mortality in patients with knee or hip osteoarthritis: population based cohort study,” Bmj (2011) 342:d1165). The cost of OA to our health care system is estimated to be over $100 billion per annum (Kotlarz et al., “Insurer and out-of-pocket costs of osteoarthritis in the US: evidence from national survey data,” Arthritis and rheumatism (2009) 60(12):3546-3553). Such statistics reflect the fact that OA is both incurable and remarkably resistant to treatment. Moreover, the incidence and prevalence of OA will rise with demographic changes in western societies. Several circumstances combine to bring this about.

Most fundamentally, the etiopathophysiology of OA is poorly understood. To some degree this is a hangover from an earlier mindset in which OA was considered an ineluctable result of wear and tear, and therefore resistant to pharmacological intervention. Studies into the biology of the disease process were therefore delayed and only recently have solid therapeutic targets emerged.

The earliest and predominant symptom of OA is pain (McCarberg & Tenzer, “Complexities in the pharmacologic management of osteoarthritis pain,” Current medical research and opinion (2013) 29(5):539-548). Pain normally arises late in the disease process, by which time there is often considerable structural alteration in the affected joint, including loss of articular cartilage, sclerosis of the sub-chondral bone, the formation of osteophytes, and synovial inflammation (Loeser et al., “Osteoarthritis: a disease of the joint as an organ,” Arthritis and rheumatism (2012) 64(6):1697-1707). In knee joints, there is also meniscal damage. In the absence of disease-modifying osteoarthritis drugs (DMOADs) (Roubille et al., “New and emerging treatments for osteoarthritis management: will the dream come true with personalized medicine?,” Expert opinion on pharmacotherapy (2013) 14(15):2059-2077) that halt or reverse disease progression, present treatments are palliative. Because there is no effective way to intervene in the disease process, many patients progress to the point of needing total joint replacement surgery (Richmond, “Surgery for osteoarthritis of the knee,” Rheumatic diseases clinics of North America (2013) 39(1):203-211). While a successful procedure, this involves major, expensive surgery with extensive rehabilitation. In many cases, there is a need for revision surgery to replace a prosthetic joint that has become dysfunctional.

In the absence of DMOADs, the present standard of care is palliative. As reflected in the most recent guidelines for treating OA of the knee issued by the American College of Rheumatology (ACR) in 2012 (Hochberg et al., “American College of Rheumatology 2012 recommendations for the use of nonpharmacologic and pharmacologic therapies in osteoarthritis of the hand, hip, and knee,” Arthritis care & research (2012) 64(4):465-474) and the American Academy of Orthopaedic Surgeons (AAOS) in 2013 (Jevsevar et al., “The American Academy of Orthopaedic Surgeons evidence-based guideline on: treatment of osteoarthritis of the knee, 2nd edition,” The Journal of bone and joint surgery American (2013) 95(20):1885-1886), present approaches to treatment fall into three progressive categories.

Non-pharmacological therapy includes a range of strategies such as patient education and self-help, exercise programs and weight loss. Pharmacological therapy includes the use of acetaminophen, non-steroidal anti-inflammatory drugs (NSAIDs), opiates and the intra-articular injection of glucocorticoids or hyaluronic acid. NSAIDs bring partial relief to many patients but are associated with upper GI bleeding and kidney failure, of especial concern in the present context as many individuals with OA are elderly. The intra-articular injection of glucocorticoids brings rapid relief in many cases, but the effects usually persist for only a few weeks. Repeated injection of glucocorticoids is impractical and counter-indicated because of concerns about infection and evidence that sustained, high doses of glucocorticoids damage articular cartilage. The benefits of the intra-articular injection of hyaluronic acid (visco-supplementation) are disputed; the ACR makes no recommendation on this score, while the AAOS no longer recommends it. The intra-articular injection of mesenchymal stem cells (MSCs) and autologous blood products, such as platelet-rich plasma, is increasingly popular but not approved by the FDA for OA.

The latest recommendations from the Osteoarthritis Research Society International and European League Against Rheumatism for treatment of OA of the knee do not differ greatly from those of the ACR and AAOS.

The recommendations of the various bodies highlight the paucity of treatment options for OA and the complete lack of reliably effective pharmacologic interventions. Even when there is some response to therapy, it addresses only the signs and symptoms, not disease progression. When treatment fails to control the symptoms and progression of OA, surgical intervention may be indicated.

Arthroscopic lavage and debridement have been widely used to provide symptomatic relief, but this approach has declined following evidence that its effects are no greater than placebo. An osteotomy is sometimes performed to realign the forces in the knee joint, so that load is now born by areas of intact cartilage. This measure can provide relief for several years until the newly weight-bearing articular cartilage erodes and symptoms reappear. In general, osteotomy is viewed as a delaying tactic that buys time until the surgical implantation of a prosthetic knee joint. Many patients progress to the point of needing total joint replacement, and over 700,000 artificial knees were surgically implanted last in year in the US (Center for Disease Control: FastStats. http://wwwcdcgov/nchs/fastats/inpatient-surgeryhtm 2015). The latter statistic demonstrates very clearly the prevalence of knee OA and how little we can do about its progression.

Accordingly, one of the most common, expensive and debilitating diseases in the western world is incurable, very difficult to treat and has few therapeutic options. These circumstances reflect the urgency for alternative, new, effective treatments.

### SUMMARY

Methods for treating a subject for arthritis are provided. Aspects of the methods include administering a rs419598/rs315952/rs9005-haplotype-informed therapeutic regimen to the subject. In some instances, the methods include administering to the subject a therapeutic regimen that antagonizes interleukin-1 (IL-1) activity and/or a disease modifying osteoarthritis drug (DMOAD) if the subject has been identified as having a TTG rs419598/rs315952/rs9005 haplotype. Also provided are compositions for use in practicing the methods.

### BRIEF DESCRIPTION OF THE FIGURES

FIG. 1: Association of IL1RN TTG haplotypes with radiographic severity. Panel A: Forest plot displaying association of IL-1RN haplotypes (TTG-0 vs. TTG-½) with radiographic severity in symptomatic knee osteoarthritis patients in three cohorts. Study-specific estimates of odds ratios (OR) with 95% confidence intervals (CI) between severity of knee OA defined as KL ½ vs. KL ¾ for haplotype rs419598, rs315952 and rs9005 “T-T-G” are shown for three independent and all three combined cohort. Panel B: Association of radiographic medial joint space width (mJSW), age and IL1RN haplotypes in NYU, OA and GOGO cohorts. Influence of interleukin 1 receptor antagonist (IL1RN) haplotypes on the age relationship to mJSW of knee osteoarthritis (OA). Carriers of either TTG-1 or TTG-2 compared to TTG-0 had narrower JSW (mm) at each age (years) studied. The joint space width (JSW) of each knee in patients with knee OA who do not (TTG-0) or do carry the IL1RN TTG haplotype is plotted relative to age, and the regression line is shown for JSW relative to age. The figure shows the linear regression line for each of the IL1RN risk haplotypes. OA patients 982 out of 1066 from three cohorts are represented. Panel C: The mean (95% CI) mJSW of TTG-0/1/2 carriers at different ages are shown. Decreased mJSW is significantly associated at 60 and 70 years age (p=0.031 and 0.005 respectively) in patients with knee OA who do carry the IL1RN TTG1 or 2 haplotype relative to TTG-0.

FIG. 2: Incidence flow chart—selection and analysis of incident OA from the OAI.

FIG. 3: Causal analysis of baseline biomarkers along with age, sex and BMI on medial joint space narrowing (JSN). To determine the interdependence of IL1RN haplotype, plasma IL-1Ra biomarker, and covariates [body mass index (BMI), age, sex], on medial joint space width (mJSW). FCI algorithm was used for causal graph analysis of all variables. Edges with a single arrow denote causality, edges with double arrows denote hidden confounders, and marks (circles) on the edges denote uncertainty of causal orientation. Haplotypes TTG or CTA represents carriers of either IL1RN haplotype produced using 3 IL1RN SNPs (rs419598, rs315952 and rs9005).

FIG. 4: % EWL after bariatric surgery continues for 12 months, but knee pain levels off after one month. Patients undergoing all three types of weight loss surgery experienced weight loss throughout a 12-month period, but knee OA relief plateaued after one month. LapBand surgery had a more modest effect on the knee symptoms than anatomy-altering surgeries.

FIG. 5: IL1RN genetic polymorphism associated with significant decrease (baseline—6 months) in serum highly sensitive c-reactive protein (hsCRP) and leptin following sleeve surgery in patients with obesity. IL1RN non-TTG carriers presented with lower hsCRP and leptin at baseline relative to TTG carriers and significantly decreased (y-axis) following surgery over a period of time compared to TTG carriers who presented with higher hsCRP and leptin. Higher inflammation in IL1RN TTG carriers associated with relatively less weight loss following bariatric surgery.

### DETAILED DESCRIPTION

Methods for treating a subject for arthritis are provided. Aspects of the methods include administering a rs419598/rs315952/rs9005-haplotype-informed therapeutic regimen to the subject. In some instances, the methods include administering to the subject a therapeutic regimen that antagonizes interleukin-1 (IL-1) activity and/or a disease modifying osteoarthritis drug (DMOAD) if the subject has been identified as having a TTG rs419598/rs315952/rs9005 haplotype. Also provided are compositions for use in practicing the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, the present disclosure provides methods for treating a subject, such as a mammal, e.g., a human, suffering from arthritis. The term “arthritis” is employed collectively to refer to diseases characterized by swelling and tenderness of one or more joints. Arthritis conditions include: Ankylosing spondylitis; Gout; Joint infections; Juvenile idiopathic arthritis; Osteoarthritis; Psoriatic arthritis; Reactive arthritis; Rheumatoid arthritis; Septic arthritis and Thumb arthritis.

The term “osteoarthritis” is used in its conventional sense to refer to type of arthritis caused by breakdown and eventual loss of cartilage in the joints. Osteoarthritis (OA) is also known as degenerative arthritis and degenerative joint disease. Clinically, OA is characterized by articular cartilage degradation followed by joint space narrowing. Multiple causative factors have been implicated, including: joint trauma, congenital dysplasia and aging. OA is also thought of as a disease that can occur insidiously during aging. Regardless of the underlying cause, the clinical findings in patients with OA are almost universal. Patients typically complain of pain, stiffness, decreased range of motion, palpable grinding within the joint (crepitus), swelling and eventual joint enlargement or deformity. Macroscopically, the articular cartilage surface develops areas of focal damage and softening early in the disease process. As OA progresses, surface cartilaginous fibrillations and vertical clefts develop, and eventually there are large areas of full thickness cartilage loss with exposed, eburnated subchondral bone. Radiographically, this process is seen as progressive joint space narrowing (secondary to loss of the radiolucent articular cartilage), subchondral bony sclerosis and cyst formation, and the development of marginal osteophytes. Eventually, the cumulative effect of all of these changes leads to decreased use of the joint, muscular atrophy, and debilitating pain (Felson et al., (2000) Ann. Intern. Med., 133(8):635-646). Microscopically, the synovial and cartilaginous tissues undergo characteristic changes as OA progresses. These articular tissues show significantly increased cellular proliferation. Either before or concomitant with the development of surface fibrillations, the macromolecular framework of the matrix is disrupted, and the water content increases. This is accompanied by a decrease in the aggregation of proteoglycans, the concentration of aggrecans, and the length of the glycosaminoglycan chains. These changes lead to an increase in the overall permeability of the matrix which decreases the cartilage stiffness and makes it more susceptible to further biochemical and biomechanical damage.

At the molecular level, cartilage matrix degradation is orchestrated by immune and inflammatory signals. Multiple molecular players, including inflammatory cytokines such as IL-1 and TNF, and, matrix metalloproteinases, such as MMP-2, 9 and 13 and aggrecanases: ADAMTS4 and 5 have been implicated in this degradative process. Cascades of inflammatory cytokines and catabolic enzymes are released from the cells in the synovium to orchestrate cartilage degradation. Regardless of the initiating etiological factors, the events producing the pathological changes involve a cascade of biological processes (Malemud et al., (2003) Cells Tissues Organs, 174:34-48).

The OA that is treated by the methods described herein may vary. While the OA may be associated with any joint, in some instances the joint is OA of the hand, knee, hip, shoulder, ankle, elbow, temperomandibular joint, and spine, and combinations thereof. In some instances, the OA that is treated by methods as described herein is OA of the knee.

Rheumatoid arthritis (RA) (also known as inflammatory joint disease) is an autoimmune disorder that causes chronic inflammation of the synovium, the lining of the membrane that surrounds joints (Mayo Clinic, Rheumatoid arthritis, 2014). The inflammation in RA causes swelling that can result in bone erosion, joint deformity, and pain. Id. RA may also affect other parts of the body, such as the eyes, lungs, blood vessels, and skin and eventually lead to osteoporosis, carpal tunnel syndrome, hardened and blocked arteries, inflammation of the sac that encloses the heart, and inflammation and scarring of lung tissue. Id. Some symptoms of RA include fatigue, fever, weight loss, bumps of tissue under the skin of the arms, stiffness in the joints in the morning that may last for hours, and joints that are tender, warm, and swollen. Id.

By “treatment” it is meant that at least an amelioration of one or more symptoms, e.g., pain, associated with arthritis, e.g., OA, is achieved, where amelioration is used in a broad sense to refer to at least a reduction in the magnitude of a parameter, e.g., a symptom associated with the arthritis, e.g., OA. As such, treatment also includes situations where a pathological condition, or at least symptoms associated therewith, are completely inhibited, e.g., prevented from happening, or stopped, e.g., terminated, such that the human no longer suffers from arthritis, e.g., OA, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of arthritis, e.g., OA in the subject, and includes: (a) preventing the arthritis, e.g., OA from occurring in the subject which may be predisposed to the disease but has not yet been diagnosed as having it; (b) inhibiting the arthritis, e.g., OA, i.e., arresting its development; or (c) relieving the OA, i.e., causing regression of the arthritis, e.g., OA. Treatment may result in a variety of different physical manifestations, e.g., reduction in perceived pain, modulation of joint structure, etc. Treatment of ongoing arthritis, e.g., OA, where the treatment stabilizes or reduces the undesirable clinical symptoms of the patient, occurs in some embodiments. Such treatment may be performed prior to complete loss of function in the affected tissues. The subject therapy may be administered prior to the symptomatic state of the disease, during the symptomatic stage of the disease, and in some cases after the symptomatic stage of the disease.

As summarized above, aspects of the methods include administration to the subject a rs419598/lrs315952/rs9005-haplotype-informed therapeutic regimen, i.e. administering a rs419598/Irs315952/rs9005-haplotype-informed therapeutic regimen to the subject. In other words, the methods include treating the subject based on the rs419598/lrs315952/rs9005 haplotypes of the IL1RN gene that they carry. As such, a specific treatment regimen is administered to the subject in view of the nucleotides present at each of the rs419598, rs315952 and rs9005 single nucleotide polymorphisms (SNPs) of each allele, which are all present on the interleukin 1 receptor antagonist (IL1RN) gene. “rs419598)” means a single nucleotide polymorphism in the interleukin 1 receptor antagonist (IL1RN) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=419598). “(rs315952)” means a single nucleotide polymorphism in the interleukin 1 receptor antagonist (IL1RN) gene. This is a C/T nucleotide substitution. The sequence surrounding this SNP is available from the dbSNP database of the National Center for Biotechnology (www.ncbi.nlm.nih.gov/projects/SNP/snp_ref.cgi?rs=315952). “rs9005” means a single nucleotide polymorphism in the interleukin 1 receptor antagonist (IL1RN) gene. This is an A/G nucleotide substitution. The sequence surrounding this SNP is available from the dbSNP database of the National Center for Biotechnology (www.ncbi.nlm.nih.gov/projects/SNP/snp_ref.cgi?rs=9005).

In some embodiments, a TTG indicated treatment regimen is administered to the subject if the subject carries a TTG rs419598/rs315952/rs9005 haplotype. The TTG indicated treatment regimen may be administered to the subject if the subject is heterozygous (i.e., TTG1) or homozygous (TTG2) for the TTG rs419598/rs315952/rs9005 haplotype. In some instances, the TTG indicated treatment regimen is not administered to the subject if the subject has a CTA rs419598/rs315952/rs9005 haplotype. In such instances, even if the subject is TTG1, if the subject is also CTA1, the TTG indicated treatment regimen is not administered to the subject. Instead, another treatment regimen may be administered to the subject.

The rs419598/rs315952/rs9005 haplotypes carried by a subject may be determined using any convenient protocol. A subject may have been identified as carrying or not carrying a TTG rs419598/rs315952/rs9005 haplotype using any convenient protocol. Protocols of interest include genotyping protocols that determine a subject's nucleotides at each of the rs419598, rs315952 and rs9005 ILRN SNPs on each allele, as well as protocols that infer the subject's nucleotides at each of the rs419598, rs315952 and rs9005 ILRN SNPs based on one or more phenotypic parameters of the subject.

### Genopyting

Where genotyping protocols are employed, haplotype patterns can be identified by detecting any of the component alleles using any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments generated by endonuclease digestion). The allele can optionally be subjected to an amplification step prior to performance of the detection step. Amplification methods that may be employed include those selected from the group consisting of: the polymerase chain reaction (PCR), the ligase chain reaction (LCR), strand displacement amplification (SDA), cloning, and variations of the above (e.g. RT-PCR and allele specific amplification).

A variety of methods are available for detecting the presence of a particular single nucleotide polymorphic allele in an individual. Methods for genotyping using for example allele-specific PCR, allele-specific probe hybridization, restriction fragment length polymorphism and/or DNA sequencing may be employed. Moreover, the method for determining the identity of the allele(s) at each of said positions of SNP of the subject may advantageously comprise a multiplex method wherein two or more SNPs are analyzed in parallel. This provides efficiency savings, not least in time and sample processing. In some cases, determining the identity of at least one allele at each of said positions of SNP of the subject includes amplification, hybridization, allele-specific PCR, array analysis, bead analysis, primer extension, restriction analysis and/or sequencing. Such methods may include, but are not limited to, polymerase-based assays such as qPCR, RT-PCR (e.g., TAQMAN or SYBR Green), hybridization-based assays such as DNA microarray analysis, flap-endonuclease-based assays (e.g., INVADER), and direct mRNA capture (QUANTIGENE or HYBRID CAPTURE (Digene)). See, for example, US 2010/0190173 for descriptions of representative methods that can be used to determine expression levels.

Techniques that may be employed include, but are not limited to, dynamic allele-specific hybridization (DASH), microplate array diagonal gel electrophoresis (MADGE), pyrosequencing, oligonucleotide-specific ligation, the TaqMan™ system as well as various DNA “chip” technologies such as the Affymetrix™ SNP chips. These methods may employ amplification of the target genetic region, such as by PCR. Other methods based on the generation of small signal molecules by invasive cleavage followed by mass spectrometry or immobilized padlock probes and rolling-circle amplification may be employed. Several of the methods known in the art for detecting specific single nucleotide polymorphisms are summarized below. The present invention is understood to include all available methods for determining the haplotype pattern of the subject.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In some instances, a TaqMan allelic discrimination assay is employed. This assay makes use of the 5-exonuclease activity of a DNA polymerase to generate a signal by digesting a probe molecule to release a fluorescently labeled nucleotide. This assay is, frequently referred to as a TaqMan assay (see, e.g., Arnold, et al., BioTechniques 25(1):98-106 (1998); and Becker, et al., Hum. Gene Ther. 10:2559-66 (1999)). In these embodiments, DNA from the subject is amplified in the presence of a probe molecules that hybridize to the rs419598/lrs315952/rs9005 SNP sites. The probe molecules contain both a fluorescent reporter-labeled nucleotide at the 5′-end and a quencher-labeled nucleotide at the 3′-end. The probe sequence is selected so that the nucleotide in the probe that aligns with the SNP site in the target DNA is as near as possible to the center of the probe to maximize the difference in melting temperature between the correct match probe and the mismatch probe. As the PCR reaction is conducted, the correct match probe hybridizes to the SNP site in the target DNA and is digested by the Taq polymerase used in the PCR assay. This digestion results in physically separating the fluorescent labeled nucleotide from the quencher with a concomitant increase in fluorescence. The mismatch probe does not remain hybridized during the elongation portion of the PCR reaction and is, therefore, not digested and the fluorescently labeled nucleotide remains quenched. In these assays, any convenient probes may be employed. Suitable probes include, but are not limited to: TaqMan rs419598 (C_8737990_10): C vs. T; TaqMan rs315952 (C_11512470_10): TaqMan C vs. T; and TaqMan rs9005 (C_3133528_10): A vs. G; (ThermoScientific). Fluorescence may be detected and processed using any convenient system, e.g., ABI Prism 7900HT Detection System, and the resultant data analyzed using any convenient genotyping software, e.g., TaqMan Genotyper Software version 1.4.

In another embodiment, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™, is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Primer-guided nucleotide incorporation procedures for assaying polymorphic sites in DNA may also be employed, such as those described in (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

In one illustrative embodiment, the haplotype detection may include the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one target allele under conditions such that hybridization and amplification of the allele occurs, and (iv) detecting the amplification product. In one embodiment, the target alleles are identified by alterations in restriction enzyme cleavage patterns. For example, sample and control DNA is isolated, amplified (optionally), digested with one or more restriction endonucleases, and fragment length sizes are determined by gel electrophoresis.

Alternatively, any of a variety—of sequencing reactions known in the art can be used to directly sequence the allele. Exemplary sequencing reactions include those based on techniques developed by Maxim and Gilbert ((1977) Proc. Natl Acad Sci USA 74:560) or Sanger (Sanger et al (1977) Proc. Nat. Acad. Sci USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out. Where sequencing is employed, next generation sequencing (NGS) protocols may be used. NGS sequencing protocols of interest include those provided by Illumina® (e.g., the NovaSeq, NexSeq, HiSeq, MiSeq and/or Genome Analyzer sequencing systems); Thermo Fisher (e.g., Ion Torrent (such as the Ion PGM and/or Ion Proton sequencing systems) and Life Technologies (such as a SOLiD sequencing system)); Pacific Biosciences (e.g., the PACBIO RS II sequencing system); Roche (e.g., the 454 GS FLX+ and/or GS Junior sequencing systems); Oxford Nanopore technologies (e.g., MinION, GridION, PrometION sequencing systems); and the like.

In yet other protocols, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; and Saleeba et al (1992) Methods Enzymol. 217:286-295. In a preferred embodiment, the control DNA or RNA can be labeled for detection.

In yet other instances, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of an IL-1 locus haplotype is hybridized to a CDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 1 1:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of a target haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

For genotyping protocols, any convenient sample (e.g. a biological liquid, cell or tissue sample) that has been obtained and/or isolated from the subject may be employed. In some cases where the genotyping is performed, the method may additionally include a preceding step of obtaining a sample, in particular a DNA-containing sample, from the subject. In some cases in accordance with the method of this and other aspects of the invention the sample is selected from the group consisting of: blood, skin cells, cheek cells, saliva, hair follicles, and tissue biopsy. In some instances, peripheral blood leukocytes (PBL) are employed as a source of nucleic acid to be used in assays. PBLs can be obtained from an individual in the form of a Peripheral Blood Mononuclear Cell (PBMC) sample. PBMCs are a mixture of monocytes and lymphocytes, and there are a number of known methods for isolating PBMCs from whole blood. While any suitable method may be employed, in one embodiment, PBMCs are isolated from whole blood samples using density gradient centrifugation. Alternatively, PBL may be further isolated from whole blood or PBMCs to yield a cell subpopulation, such as a population of lymphocytes (e.g., T-lymphocytes or sub-population thereof). Examples for isolating such sub-populations include cell sorting or cell-capturing using antibodies to particular cell-specific markers. In another embodiment, PBL can be obtained from whole blood using the PAXgene kit (Qiagen).

Genotyping assays that may be employed in embodiments of methods of the invention are further described in published United States Patent Application Nos. 20180223362; 20160340734; 20160032386; 20150203917; 20150072364; the disclosures of which are herein incorporated by reference.

### Phenotyping

As indicated above, the presence of a target haplotype may also be inferred in embodiments of the methods from one or more phenotypes of the subject. The term phenotype as used herein refers to an observable characteristic of the subject. Phenotypes that may be employed to infer the presence of the target haplotype include, but are not limited to: inefficiently exported IL-1Ra; faster disease progression as compared to a suitable control; and the like.

In some instances, a subject is assayed for the phenotype of reduced or decreased soluble IL-1Ra (sIL-1Ra), such as sIL-1Ra that is inefficiently exported from chondrocytes, which phenotype may be employed in some instances to infer the presence of the a IL1RN gene TTG rs419598/rs315952/rs9005 haplotype. The assay employed may assess levels of sIL-1Ra from chondrocytes because of isotype switching, which results in relatively more intracellular IL-1Ra as compared to secreted IL-1Ra. Whether a subject has a phenotype characterized by such reduced synthesis of sIL-1Ra may be assayed using any convenient protocol. A subject will also be assayed for reduced levels of sIL1RAin blood (non-fasting plasma or serum) samples using any convenient protocol.

Detection of sIL-1Ra may be accomplished by several different techniques, many of which are antibody-based. Additional information regarding the methods discussed below may be found in Ausubel et al., (2003) CURRENT PROTOCOLS IN MOLECULAR BIOLOGY, John Wiley & Sons, New York, N.Y., or Sambrook et al. (1989) MOLECULAR CLONING: A LABORATORY MANUAL, Cold Spring Harbor Press, Cold Spring Harbor, N.Y. One skilled in the art will know which parameters may be manipulated to optimize detection of the protein of interest.

An enzyme-linked immunosorbent assay (ELISA) may be used to detect and quantify protein levels. This method comprises preparing the antigen (i.e., protein of interest), coating the wells of a microtiter plate with the antigen, incubating with an antibody that recognizes the antigen, washing away the unbound antibody, and detecting the antibody-antigen complex. The antibody is generally conjugated to an enzyme, such as horseradish peroxidase or alkaline phosphatase, which generate colorimetric, fluorescent, or chemiluminescent products. An ELISA may also use two antibodies, one of which is specific to the protein of interest and the other of which recognizes the first antibody and is coupled to an enzyme for detection. Further, instead of coating the well with the antigen, the antibody may be coated on the well. In this case, a second antibody conjugated to a detectable compound is added following the addition of the antigen of interest to the coated well.

The Luminex platform (available from Luminex Corp., Austin, Tex.) can be used to detect and quantify protein levels using multiplexed assays based on a capture bead system in which microsphere beads are color-coded with dyes into up to one hundred distinct sets. Each color-coded bead set is coated with a specific binding reagent such as an antibody specific to a selected protein marker, allowing the capture and detection of specific protein analytes from a very small amount, e.g. a drop of fluid, from a biological sample such as plasma, serum, lysates or synovial fluid. Depending upon which analyte(s) are being screened, at least one or several bead sets may be incubated with the sample in order to capture the analytes. A Luminex compact analyzer uses lasers to excite the internal dyes that identify each microsphere beads, and also any reporter dye captured during the assay. Multiple readings can be made on each bead set. Because of the special dye ratio incorporated each bead, each unique bead population can be analyzed separately after acquisition. An exemplary multiplex immunoassay platform is also the xMAP platform available from Qiagen Inc.

Relative protein levels may also be measured by Western blotting. Western blotting generally comprises preparing protein samples, using gel electrophoresis to separate the denatured proteins by mass, and probing the blot with antibodies specific to the protein of interest. Detection is usually accomplished using two antibodies, the second of which is conjugated to an enzyme for detection or another reporter molecule. Methods used to detect differences in protein levels include colorimetric detection, chemiluminescent detection, fluorescent detection, and radioactive detection.

Measurement of protein levels may also be performed using a protein microarray or an antibody microarray. In these methods, the proteins or antibodies are covalently attached to the surface of the microarray or biochip. The protein of interest is detected by interaction with an antibody, and the antibody/antigen complexes are generally detected via fluorescent tags on the antibody.

Relative protein levels may also be assessed by immunohistochemistry, in which a protein is localized in cells of a tissue section by its interaction with a specific antibody. The antigen/antibody complex may be visualized by a variety of methods. One or two antibodies may be used, as described above for ELISA. The detection antibody may be tagged with a fluorophore, or it may be conjugated to an enzyme that catalyzes the production of a detectable product. The labeled complex is typically visualized under a microscope.

The sample employed in such phenotypic assays may be derived from any biological source and in some instances is a biological fluid likely to contain exported IL-1RA. Examples of samples that may be employed include, but are not limited to, whole blood, serum, plasma, interstitial fluid, synovial fluid, peritoneal fluid, etc., where in some instances the sample is a synovial fluid sample. The sample may be used directly as obtained from the subject or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. If such methods of pretreatment are employed with respect to the test sample, such pretreatment methods are such that the analyte of interest remains in the test sample at a concentration proportional to that in an untreated test sample (e.g., namely, a test sample that is not subjected to any such pretreatment method(s)).

The method may further include comparing the measured sIL-1Ra level to a control, wherein a difference between the measured level and the control is indicative of the haplotype carried by the subject. In such embodiments, the measured sIL-1Ra level determined for a biological sample obtained from the subject is compared to the levels in one or more control samples. The control samples may be obtained from a healthy subject (or a group of healthy subjects), from a subject (or group of subjects) with OA, from a subject (or group of subjects) with subtype I OA or subtype II OA, from a subject with early OA (e.g., KL1 or KL2), from a subject with late OA (e.g., KL3 or KL4) and/or from a subject (or group of subjects) with a specific stage of the disease (e.g., early OA or late OA). In some instances, the control is one that is obtained from subjects that have been characterized for the haplotype of interest. The control expression levels of IL-1Ra may be determined from a significant number of individuals, and an average or mean is obtained. In some instances, a subject is identified has having a phenotype characterized by inefficiently exported IL-1Ra if the measured level, as compared to a suitable control, such as the level of a health individual, is 70% of the control value, or less, such as 50% of the control or less, including 10% or less, 20% or less, 30% or less, or 40% or less the control.

As indicated above, reduced levels or synthesis of IL-1Ra may result from IL-1Ra that is inefficiently exported by chondrocytes sIL-1Ra may be assayed using any convenient protocol. In one embodiment, chondrocytes may be obtained from the subject, cultured, and then intracellular vs. extracellular IL1Ra determined, e.g., using the protocols described above. In another embodiment, fibroblast cells may be obtained from the subjected, PS cells derived therefrom, followed by derivation of chondrocytes, and then intracellular vs. extracellular IL1Ra determined, e.g., using the protocols described above. In yet another embodiment, the subject's IL-1Ra and surrounding region may be sequenced, cloned into a vector, which vector is then employed to transduce a chondrocyte model system, and then intracellular vs. extracellular IL-1Ra determined, e.g., using the protocols described above. In yet another embodiment, the subject's IL-1Ra may be sequenced and compared to a database with known reference samples that match the sequence, followed by correlation of the results with the intracellular vs. extracellular IL1 Ra in the database.

As summarized above, another phenotype that may be employed to infer the presence of a TTG rs419598/rs315952/rs9005 haplotype is faster disease progression as compared to a suitable control. Faster disease progression as compared to a suitable control may be determined using any convenient protocol. In some instances, disease progression is assessed at least in part using an imaging protocol. A variety of different imaging protocols may be employed in assessing OA.

In some instances, a radiographic (i.e., X-ray) protocol is employed. For example, the structural progression of OA may be assessed on plain radiographic views by measuring the joint space width (JSW) and/or joint space narrowing (JSN) over a period of time. (Altman et al.: Osteoarthritis Cartilage 1996, 4:217-243.) OA progression is associated with accelerated cartilage degradation leading to JSN, painful joint disruption, and functional compromise. OA disease progression may be measured on a Kellgren-Lawrence Grading Scale (“KL scale”) to measure occurrence and severity of OA in human subjects. Grade 0 means the joints of a human subject is normal. Grade 1 means a human subject has doubtful narrowing of joint space and possible osteophytic lipping. Grade 2 means a human subject has definite osteophytes, definite narrowing of joint space. Grade 3 means a human subject has moderate multiple osteophytes, definite narrowing of joint space, some sclerosis and possible deformity of bone contour. Grade 4 means a human subject has large osteophytes, marked narrowing of joint space, severe sclerosis and definite deformity of bone contour. In some instances, the OA of the human has been assigned a Kellgren-Lawrence score of 2, 3 or 4. In some instances, the human has been assigned a Kellgren-Lawrence score of 2 or 3.

Other imaging protocols may also be employed. Other imaging protocols of interest include, but are not limited to, computed tomography imaging (CT scan), ultrasound scanning or imaging (sonography), magnetic resonance imaging (MRI), including contrast enhanced MRI, biochemical magnetic resonance imaging (e.g., T2 mapping, T1 rho imaging, sodium MRI, and delayed gadolinium-enhanced MRI of cartilage or dGEMRiC), 3-D imaging, or other medical imaging techniques.

In some instances, the disease progression is assessed using a magnetic resonance imaging (MRI) protocol (such as described above), e.g., to identify bone marrow lesions. Bone marrow lesions (or edemas) are very strongly associated with knee arthritis pain (Felson et al., “The association of bone marrow lesions with pain in knee osteoarthritis.” Ann Intern Med. 2001 Apr. 3; 134(7):541-9) and disease progression (Felson et al., “Bone marrow edema and its relation to progression of knee osteoarthritis.” Ann Intern Med. 2003 Sep. 2; 139(5 Pt 1):330-6). See also U.S. Pat. No. 6,564,083 to Stevens, which describes a method of identifying in a patient having joint pain the susceptibility of the patient to developing progressive OA or loss of joint space, by determining the presence or absence of bone marrow edema about or of the joint. The determination may be made through the use of MRI. See also U.S. Published Patent Application 20170202520 the disclosure of which is herein incorporated by reference.

Where faster disease progression is employed to infer the presence of a TTG interleukin 1 receptor antagonist (IL1RN) rs419598/IL1RN rs315952/IL1RN rs9005 haplotype, disease severity in the subject may be observed.

### Treatment

As summarized above, aspects of the methods include treating the subject based on their carriage of an interleukin 1 receptor antagonist (IL1RN) rs419598/IL1RN rs315952/IL1RN rs9005 haplotype. In some embodiments, a TTG indicated treatment regimen is administered to the subject if the subject carries a TTG IL1RN rs419598/IL1RN rs315952/IL1RN rs9005 haplotype. The TTG indicated treatment regimen may be administered to the subject if the subject is heterozygous (i.e., TTG1) or homozygous (TTG2) for the TTG IL1RN rs419598/IL1RN rs315952/IL1RN rs9005 haplotype. In some instances, the TTG indicated treatment regimen is not administered to the subject if the subject has a CTA IL1RN rs419598/IL1RN rs315952/IL1RN rs9005 haplotype. In such instances, even if the subject is TTG1, if the subject is also CTA1, the TTG indicated treatment regimen is not administered to the subject. Instead, another treatment regimen may be administered to the subject. In some instances, if the subject is TTG1/CTA0, the TTG indicated treatment regimen is administered to the subject.

In some instances, e.g., where the arthritis is osteoarthritis or rheumatoid arthritis, the TTG indicated treatment regimen that is administered to the subject if the subject carries a TTG IL1RN rs419598/IL1RN rs315952/IL1RN rs9005 haplotype is a treatment regimen that antagonizes interleukin-1 (IL-1) activity. In such embodiments, any treatment regimen that antagonizes IL-1 activity may be employed. A treatment regimen that antagonizes IL-1 activity may include administered an interleukin-1 antagonist/inhibitor (i.e., inhibitor of IL-1) to the subject. The term “inhibitor of IL-1” within the context of the application refers to any agent that modulates IL-1 production and/or action in such a way that IL-1 production and/or action is attenuated, reduced, or partially, substantially or completely prevented or blocked. The term “IL-1 antagonist/inhibitor” is meant to encompass inhibitors of IL-1 production as well as of inhibitors of IL-1 action. An inhibitor of production can be any agent negatively affecting the synthesis, processing or maturation of IL-1. The inhibitors considered according to the invention can be, for example, suppressors of gene expression of the interleukin IL-1, antisense mRNAs reducing or preventing the transcription of the IL-1 mRNA or leading to degradation of the mRNA, proteins impairing correct folding, or partially or substantially preventing secretion of IL-1, proteases degrading IL-1, once it has been synthesized, inhibitors of proteases cleaving pro-IL-1 in order to generate mature IL-1, such as inhibitors of caspase-1, and the like. An inhibitor of IL-1 action can be an IL-1 antagonist, for example. Antagonists can either bind to or sequester the IL-1 molecule itself with sufficient affinity and specificity to partially or substantially neutralize the IL-1 or IL-1 binding site(s) responsible for IL-1 binding to its ligands (like, e.g. to its receptors). An antagonist may also inhibit the IL-1 signaling pathway, which is activated within the cells upon IL-1/receptor binding. Inhibitors of IL-1 action may be also soluble IL-1 receptors or molecules mimicking the receptors (e.g., IL-1 soluble receptor), or agents blocking the IL-1 receptors (e.g., IL-1 receptor antagonist such as IL-1Ra, or IL-1 receptor antibody), or IL-1 antibodies, such as polyclonal or monoclonal antibodies (e.g., antibodies or binding fragments thereof that bind to IL-1α or IL-1β, or any other agent or molecule preventing the binding of IL-1 to its targets, thus diminishing or preventing triggering of the intra- or extracellular reactions mediated by IL-1. An antagonist/inhibitor of IL-1 may be selected from small molecules, e.g., caspase-1 (ICE) inhibitors, antibodies against IL-1, antibodies against any of the IL-1 receptor subunits, inhibitors of the IL-1 signaling pathway, antagonists of IL-1 which compete with IL-1 and block the IL-1 receptor, IL-1 receptor antagonist (IL-1Ra) and IL-1 binding proteins, or an isoform, mutein, fused protein, functional derivative, active fraction or circularly permutated derivatives thereof. In some instances, the antagonist is IL-1Ra or a recombinant version thereof, or an isoform, mutein, fused protein, functional derivative, active fraction or circularly permutated derivative thereof, etc.

In some instances, the therapeutic regimen increases synovial fluid IL-1Ra concentration. Examples of such instances include methods in which a nucleic acid coding sequence for an IL-1Ra, such as human IL-1Ra, is administered to the subject so as to treat the subject for OA. In some embodiments, a dosage of the coding sequence is intra-articularly administered to the subject, such that administration of the dosage results in the dosage being situated within a joint, e.g., at a synovial location, such as where the dosage is administered by entering a joint. The dosage may be intra-articularly administered using any convenient protocol, e.g., via delivery through a needle where the distal end has been positioned at the within the target joint, e.g., where the distal end of the needle is positioned at a synovial location such that delivery of the dosage out the distal end of the needle results in delivery of the dosage to the target joint. In some instances, the target joint is a joint of the hand, knee, hip, shoulder, ankle, elbow, temperomandibular joint, or spine, and in some instances is a knee joint.

The administered dosage includes a nucleic acid coding sequence for a IL-1Ra (gene IL1RNV). IL-1Ra is a protein that binds to IL-1 receptors and inhibits the binding of IL-1alpha and IL-1beta thereto. IL-1Ra proteins and coding sequences therefore are further described in: international patent application serial no. PCT/US2017/065173 published as WO/2018/106956; international patent application serial no. PCT/US2017/047589 published as WO/2018/03545; international patent application serial no. PCT/US2017/047607 published as WO/2018/035457; and international patent application serial no. PCT/US2017/047572 published as WO/2018/035441; the disclosures of which are herein incorporated by reference.

In some instances, the coding sequence encodes human IL-1Ra. The canonical amino acid sequence of human IL-1Ra is:

```
(SEQ ID NO: 01)
              10 20 30 40
MEICRGLRSH LITLLLFLFH SETICRPSGR KSSKMQAFRI
              50 60 70 80
WDVNQKTFYL RNNQLVAGYL QGPNVNLEEK IDVVPIEPHA
                90 100 110 120
LFLGIHGGKM CLSCVKSGDE TRLQLEAVNI TDLSENRKQD
              130 140 150 160
KRFAFIRSDS GPTTSFESAA CPGWFLCTAM EADQPVSLTN
              170
MPDEGVMVTK FYFQEDE
```

The human IL-1Ra protein that is encoded by the administered nucleic acid may have the canonical sequence provided above, or a variant thereof. In some instances, the encoded human IL-1Ra that is administered to the human has an amino acid sequence that comprises a region substantially the same as or identical to the sequence appearing as SEQ ID NO:01. By substantially the same as is meant a protein having a region with a sequence that is 75% or greater, such as 85% or greater, such as 90% or greater and including 93% or greater, 94% or greater, 95% or greater, 96% or greater, 97% or greater, or 98% or greater sequence identity with the sequence of SED ID NO:01, as determined by BLAST using default settings.

In addition to the naturally occurring human IL-1Ra proteins, e.g., as described above, proteins that vary from the naturally occurring human IL-1Ra may also be employed in practicing methods of the invention. Different variations may be present, including but not limited to substitution, insertion and/or deletion mutations. Human IL-1Ra polypeptides that may be employed include proteins having an amino acid sequence encoded by an open reading frame (ORF) of an IL-1Ra gene, including the full-length IL-1Ra protein and fragments thereof, such as biologically active fragments and/or fragments corresponding to functional domains; and including fusions of the subject polypeptides to other proteins or parts thereof.

Fragments of interest may vary in length, and in some instances are10 aa or longer, such as 50 aa or longer, and including 100 aa or longer, and in some instances do not exceed 150 aa in length, where a given fragment will have a stretch of amino acids that is substantially the same as or identical to a subsequence found in any of SEQ ID NO:01; where the subsequence may vary in length and in some instances is 10 aa or longer, such as 15 aa or longer, up to 50 aa or even longer.

In some instances, the sequence of the protein encoded by the nucleic acid is the sequence of Kineret, which is:

(SEQ ID NO: 02)
MRPSGRKSSK MQAFRIWDVN QKTFYLRNNQ LVAGYLQGPN
VNLEEKIDVV PIEPHALFLG IHGGKMCLSC VKSGDETRLQ
LEAVNITDLS ENRKQDKRFA FIRSDSGPTT SFESAACPGW
FLCTAMEADQ PVSLTNMPDE GVMVTKFYFQ EDE

In some instances, the sequence of the protein encoded by the nucleic acid is a functional fragment of the IL-1Ra protein. A functional fragment is understood to mean a part of the IL-1Ra protein that binds to the IL-1 receptor. Such a fragment would include sequences that contact the IL-1 receptor, as described in Schreuder et al., Eur J Biochem. 1995 Feb. 1; 227(3):838-47, Clancy et al. Acta Crystallogr, 1994; D50, 197-201, Vigers et al, J. Biol. Chem., 1994; 269:12874. In some instances, a functional fragment of IL-1Ra includes one or more of the five critical amino acid residues that were identified by Schreuder et al., Nature (1997); 386:194: Trp 16, Gln 20, Tyr 34, Gln 36, and Tyr 147. In some instances, a functional fragment includes amino acid residues 34-39 of SEQ. ID NO. 1, which is known to fit in the cleft between domains 1 and 2 of the IL-1 receptor. These articles are incorporated herein by reference.

In practicing methods such as described herein, any convenient IL-1Ra coding sequence that encodes the desired IL-1Ra protein, such as described above, may be employed. Depending on the desired human IL-1Ra, the nucleic acid coding sequence may vary. Nucleic acids of interest include those encoding the human IL-1Ra proteins provided above. Specific nucleic acids of interest include, but are not limited to, those assigned the following NCBI Accession Nos: XM_005263661.4; NM_000577.4; XM_011511121.1; NM_001318914.1; NM_173842.2; NM_173841.2 and NM_173843.2.

In some instances, the nucleic acids have a sequence that is 60% or more, such as 70% or more, 80% or more, 90% or more, including 95% or more, similar to:

(SEQ ID NO: 03)
atggaaatctgcagaggcctccgcagtcacctaatcactctcctcctcttc
ctgttccattcagagacgatctgccgaccctctgggagaaaatccagcaag
atgcaagccttcagaatctgggatgttaaccagaagaccttctatctgagg
aacaaccaactagttgctggatacttgcaaggaccaaatgtcaatttagaa
gaaaagatagatgtggtacccattgagcctcatgctctgttcttgggaatc
catggagggaagatgtgcctgtcctgtgtcaagtctggtgatgagaccaga
ctccagctggaggcagttaacatcactgacctgagcgagaacagaaagcag
gacaagcgcttcgccttcatccgctcagacagtggccccaccaccagtttt
gagtctgccgcctgccccggttggttcctctgcacagcgatggaagctgac
cagcccgtcagcctcaccaatatgcctgacgaaggcgtcatggtcaccaaa
ttctacttccaggaggacgag

By nucleic acid composition is meant a composition comprising a sequence of DNA having an open reading frame that encodes a human IL-1Ra protein of interest, i.e., a human IL-1Ra coding sequence, and is capable, under appropriate conditions, of being expressed as a human IL-1Ra protein. Also encompassed in this term are nucleic acids that are homologous, substantially similar or identical to the specific nucleic acids described above. In certain embodiments, sequence similarity between homologues is 20% or higher, such as 25% or higher, and including 30%, 35%, 40%, 50%, 60%, 70% or higher, including 75%, 80%, 85%, 90% and 95% or higher. Sequence similarity is calculated based on a reference sequence, which may be a subset of a larger sequence, such as a conserved motif, coding region, flanking region, etc. A reference sequence may be 18 nt long or longer, such as 30 nt long, and may extend to the complete sequence that is being compared. Algorithms for sequence analysis are known in the art, such as BLAST, described in Altschul et al. (1990), J. Mol. Biol. 215:403-10 (using default settings, i.e. parameters w=4 and T=17). Of particular interest in certain embodiments are nucleic acids of substantially the same length as specific human IL-1Ra nucleic acids mentioned above, where by substantially the same length is meant that any difference in length in terms of number of residues does not exceed about 20%, usually does not exceed about 10% and more usually does not exceed about 5%; and have sequence identity to any of these sequences of at 80% or greater, 85% or greater, 90% or greater, such as 95% or greater and including 99% or greater over the entire length of the nucleic acid. In some embodiments, the nucleic acids have a sequence that is substantially similar or identical to the above specific sequences. By substantially similar is meant that sequence identity is 60% or greater, such as 75% or greater and including 80, 85, 90, or even 95% or greater. Nucleic acids of interest also include nucleic acids that encode the proteins encoded by the above described nucleic acids, but differ in sequence from the above described nucleic acids due to the degeneracy of the genetic code. The employed coding sequence may or may not be naturally occurring.

In some instances, the coding sequence is one that is codon-optimized. A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein. A codon optimized coding sequence of interest includes:

(SEQ ID NO: 04)
ATGGAAATCTGCAGAGGCCTGCGGAGCCACCTGATTACCCTGCTGCTGTTC
CTGTTCCACAGCGAGACAATCTGCCGGCCCAGCGGCCGGAAGTCCAGCAAG
ATGCAGGCCTTCCGGATCTGGGACGTGAACCAGAAAACCTTCTACCTGCGG
AACAACCAGCTGGTGGCCGGATACCTGCAGGGCCCCAACGTGAACCTGGAA
GAGAAGATCGACGTGGTGCCCATCGAGCCCCACGCCCTGTTTCTGGGCATC
CACGGCGGCAAGATGTGCCTGAGCTGCGTGAAGTCCGGCGACGAGACAAGA
CTGCAGCTGGAAGCCGTGAACATCACCGACCTGAGCGAGAACCGGAAGCAG
GACAAGAGATTCGCCTTCATCAGAAGCGACAGCGGCCCCACCACCAGCTTT
GAGAGCGCCGCCTGCCCCGGCTGGTTCCTGTGTACAGCCATGGAAGCCGAC
CAGCCCGTGTCCCTGACAAACATGCCCGACGAGGGCGTGATGGTCACCAAG
TTCTATTTTCAAGAAGATGAGTAA
or
(SEQ ID NO: 05)
ATGGAAATTTGCCGCGGCCTGCGCAGCCATCTGATTACCCTGCTGCTGTTT
CTGTTTCATAGCGAAACCATTTGCCGCCCGAGCGGCCGCAAAAGCAGCAAA
ATGCAGGCGTTTCGCATTTGGGATGTGAACCAGAAAACCTTTTATCTGCGC
AACAACCAGCTGGTGGCGGGCTATCTGCAGGGCCCGAACGTGAACCTGGAA
GAAAAAATTGATGTGGTGCCGATTGAACCGCATGCGCTGTTTCTGGGCATT
CATGGCGGCAAAATGTGCCTGAGCTGCGTGAAAAGCGGCGATGAAACCCGC
CTGCAGCTGGAAGCGGTGAACATTACCGATCTGAGCGAAAACCGCAAACAG
GATAAACGCTTTGCGTTTATTCGCAGCGATAGCGGCCCGACCACCAGCTTT
GAAAGCGCGGCGTGCCCGGGCTGGTTTCTGTGCACCGCGATGGAAGCGGAT
CAGCCGGTGAGCCTGACCAACATGCCGGATGAAGGCGTGATGGTGACCAAA
TTTTATTTTCAGGAAGATGAA

Such a sequence may have the consensus codon sequence:

(SEQ ID NO: 06)
ATGGARATHTGYMGNGGNYTNMGNWSNCAYYTNATHACNYTNYTNYTNTTY
YTNTTYCAYWSNGARACNATHTGYMGNCCNWSNGGNMGNAARWSNWSNAAR
ATGCARGCNTTYMGNATHTGGGAYGTNAAYCARAARACNTTYTAYYTNMGN
AAYAAYCARYTNGTNGCNGGNTAYYTNCARGGNCCNAAYGTNAAYYTNGAR
GARAARATHGAYGTNGTNCCNATHGARCCNCAYGCNYTNTTYYTNGGNATH
CAYGGNGGNAARATGTGYYTNWSNTGYGTNAARWSNGGNGAYGARACNMGN
YTNCARYTNGARGCNGTNAAYATHACNGAYYTNWSNGARAAYMGNAARCAR
GAYAARMGNTTYGCNTTYATHMGNWSNGAYWSNGGNCCNACNACNWSNTTY
GARWSNGCNGCNTGYCCNGGNTGGTTYYTNTGYACNGCNATGGARGCNGAY
CARCCNGTNWSNYTNACNAAYATGCCNGAYGARGGNGTNATGGTNACNAAR
TTYTAYTTYCARGARGAYGAR

In some embodiments, the nucleic acid sequence contains specific nucleic acids corresponding to rs419598 and rs315952, and in some instances rs419598 “C” (not “T”) and rs315952 “C” (not “T”).

Nucleic acids as described herein may be present in a vector. Various vectors (e.g., viral vectors, bacterial vectors, or vectors capable of replication in eukaryotic hosts) can be used in accordance with the present invention. Numerous vectors which can replicate in eukaryotic hosts are known in the art and are commercially available. In some instances, such vectors used in accordance with the invention are composed of a bacterial origin of replication and a eukaryotic promoter operably linked to the coding sequence of interest.

Viral vectors used in accordance with the invention may be composed of a viral particle derived from a naturally-occurring virus which has been genetically altered to render the virus replication-defective and to express a recombinant gene of interest in accordance with the invention. Once the virus delivers its genetic material to a cell, it does not generate additional infectious virus but does introduce exogenous recombinant genes into the cell, and in some instances into the genome of the cell. Numerous viral vectors are known in the art, including, for example, retrovirus, adenovirus, helper-dependent adenovirus, adeno-associated virus (AAV), herpes simplex virus (HSV), cytomegalovirus (CMV), vaccinia and poliovirus vectors, lentivirus, poxvirus, hemagglutinatin virus of Japan-liposome (HVJ) complex, Moloney murine leukemia virus, and HIV-based virus. In some instances, the vector that is employed is a non-integrating vector.

In some embodiments, the employed vector is the AAV, which is a small, non-pathogenic dependovirus that has not been associated with human disease, and in the absence of co-infection with a helper virus such as adenovirus or HSV, is unable to replicate. AAV virions, which are non-enveloped and measure 25 nm in diameter, have a genome of 4.9 kB. The AAV genome, which is single-stranded DNA, consists of three open reading frames (ORFs) flanked by two inverted terminal repeats (ITRs), which are 145 bp palendromic sequences that form elaborate hairpin structures and are essential for viral packaging. The first ORF is rep, which encodes 4 proteins involved in viral replication (Rep40, Rep52, Rep68, and Rep72). The second ORF contains cap, which encodes the three structural proteins that make up the icosahedral AAV capsid (VP1, VP2, and VP3). A third ORF, which exists as a nested alternative reading frame in the cap gene, encodes the assembly-activating protein, which localizes AAV capsid proteins to the nucleolus and participates in the process of capsid assembly. AAV has proven to be a safe and efficient vehicle for delivering therapeutic DNA to numerous tissue targets.

Gene delivery vehicles or vectors based on AAV offer many advantages over other viruses. AAV vectors have the ability to infect quiescent cells and give rise to long-term expression of transgenes, and various serotypes exhibit tropisms for different subsets of cells. The delivery efficacy or tropism for different cells depends on a combination of the capsid and the route of administration, which can be either intravenous to expose virus to the body including multiple joints, or intra-articular to expose virus primarily to the injected joint.

Next generation AAV vectors include, for example, self-complementary vectors (scAAV), whose genomes contain both a sense copy of the transgene and a reverse complement, separated by a linker. These two copies are able to anneal and serve as a double stranded template that can be transcribed without the need for generation of any complementary strand by the host cell. scAAV2, scAAV5 and scAAV8 are specific examples of such vectors.

Specific AAV vectors finding use in embodiments of the invention include, but are not limited to: AAV1, AAV2, AAV2.5, AAV3, AAV4, AAV5, AAV6, AAV7, AAV8, AAV9, AAV10, AAV11, AAV12 and Anc80.

In some instances, the vector encodes a viral cap
gene and has a sequence that is the same as
SEQ ID NO: 07
ATGGCTGCCGATGGTTATCTTCCAGATTGGCTCGAGGACACTCTCTCTGAA
GGAATAAGACAGTGGTGGAAGCTCAAACCTGGCCCACCACCACCAAAGCCC
GCAGAGCGGCATAAGGACGACAGCAGGGGTCTTGTGCTTCCTGGGTACAAG
TACCTCGGACCCTTCAACGGACTCGACAAGGGAGAGCCGGTCAACGAGGCA
GACGCCGCGGCCCTCGAGCACGACAAAGCCTACGACCGGCAGCTCGACAGC
GGAGACAACCCGTACCTCAAGTACAACCACGCCGACGCGGAGTTTCAGGAG
CGCCTTAAAGAAGATACGTCTTTTGGGGGCAACCTCGGACGAGCAGTCTTC
CAGGCGAAAAAGAGGGTTCTTGAACCTCTGGGCCTGGTTGAGGAACCTGTT
AAGACGGCTCCGGGAAAAAAGAGGCCGGTAGAGCACTCTCCTGTGGAGCCA
GACTCCTCCTCGGGAACCGGAAAGGCGGGCCAGCAGCCTGCAAGAAAAAGA
TTGAATTTTGGTCAGACTGGAGACGCAGACTCAGTACCTGACCCCCAGCCT
CTCGGACAGCCACCAGCAGCCCCCTCTGGTCTGGGAACTAATACGATGGCT
ACAGGCAGTGGCGCACCAATGGCAGACAATAACGAGGGCGCCGACGGAGTG
GGTAATTCCTCGGGAAATTGGCATTGCGATTCCACATGGATGGGCGACAGA
GTCATCACCACCAGCACCCGAACCTGGGCCCTGCCCACCTACAACAACCAC
CTCTACAAACAAATTTCCAGCCAATCAGGAGCCTCGAACGACAATCACTAC
TTTGGCTACAGCACCCCTTGGGGGTATTTTGACTTCAACAGATTCCACTGC
CACTTTTCACCACGTGACTGGCAAAGACTCATCAACAACAACTGGGGATTC
CGACCCAAGAGACTCAACTTCAAGCTCTTTAACATTCAAGTCAAAGAGGTC
ACGCAGAATGACGGTACGACGACGATTGCCAATAACCTTACCAGCACGGTT
CAGGTGTTTACTGACTCGGAGTACCAGCTCCCGTACGTCCTCGGCTCGGCG
CATCAAGGATGCCTCCCGCCGTTCCCAGCAGACGTCTTCATGGTGCCACAG
TATGGATACCTCACCCTGAACAACGGGAGTCAGGCAGTAGGACGCTCTTCA
TTTTACTGCCTGGAGTACTTTCCTTCTCAGATGCTGCGTACCGGAAACAAC
TTTACCTTCAGCTACACTTTTGAGGACGTTCCTTTCCACAGCAGCTACGCT
CACAGCCAGAGTCTGGACCGTCTCATGAATCCTCTCATCGACCAGTACCTG
TATTACTTGAGCAGAACAAACACTCCAAGTGGAACCACCACGCAGTCAAGG
CTTCAGTTTTCTCAGGCCGGAGCGAGTGACATTCGGGACCAGTCTAGGAAC
TGGCTTCCTGGACCCTGTTACCGCCAGCAGCGAGTATCAAAGACATCTGCG
GATAACAACAACAGTGAATACTCGTGGACTGGAGCTACCAAGTACCACCTC
AATGGCAGAGACTCTCTGGTGAATCCGGGCCCGGCCATGGCAAGCCACAAG
GACGATGAAGAAAAGTTTTTTCCTCAGAGCGGGGTTCTCATCTTTGGGAAG
CAAGGCTCAGAGAAAACAAATGTGGACATTGAAAAGGTCATGATTACAGAC
GAAGAGGAAATCAGGACAACCAATCCCGTGGCTACGGAGCAGTATGGTTCT
GTATCTACCAACCTCCAGAGAGGCAACAGACAAGCAGCTACCGCAGATGTC
AACACACAAGGCGTTCTTCCAGGCATGGTCTGGCAGGACAGAGATGTGTAC
CTTCAGGGGCCCATCTGGGCAAAGATTCCACACACGGACGGACATTTTCAC
CCCTCTCCCCTCATGGGTGGATTCGGACTTAAACACCCTCCTCCACAGATT
CTCATCAAGAACACCCCGGTACCTGCGAATCCTTCGACCACCTTCAGTGCG
GCAAAGTTTGCTTCCTTCATCACACAGTACTCCACGGGACAGGTCAGCGTG
GAGATCGAGTGGGAGCTGCAGAAGGAAAACAGCAAACGCTGGAATCCCGAA
ATTCAGTACACTTCCAACTACAACAAGTCTGTTAATGTGGACTTTACTGTG
GACACTAATGGCGTGTATTCAGAGCCTCGCCCCATTGGCACCAGATACCTG
ACTCGTAATCTGTAA

or substantially similar thereto, where by substantially similar is meant that sequence identity is 60% or greater, such as 75% or greater and including 80, 85, 90, or even 95% or greater.

The protein sequence encoded thereby is
SEQ ID NO. 08:
MAADGYLPDWLEDTLSEGIRQWWKLKPGPPPPKPAERHKDDSRGLVLPGYK
YLGPFNGLDKGEPVNEADAAALEHDKAYDRQLDSGDNPYLKYNHADAEFQE
RLKEDTSFGGNLGRAVFQAKKRVLEPLGLVEEPVKTAPGKKRPVEHSPVEP
DSSSGTGKAGQQPARKRLNFGQTGDADSVPDPQPLGQPPAAPSGLGTNTMA
TGSGAPMADNNEGADGVGNSSGNWHCDSTWMGDRVITTSTRTWALPTYNNH
LYKQISSQSGASNDNHYFGYSTPWGYFDENREHCHFSPRDWQRLINNNWGF
RPKRLNEKLENIQVKEVTQNDGTTTIANNLTSTVQVFTDSEYQLPYVLGSA
HQGCLPPFPADVFMVPQYGYLTLNNGSQAVGRSSFYCLEYFPSQMLRTGNN
FTFSYTFEDVPFHSSYAHSQSLDRLMNPLIDQYLYYLSRTNTPSGTTTQSR
LQFSQAGASDIRDQSRNWLPGPCYRQQRVSKTSADNNNSEYSWTGATKYHL
NGRDSLVNPGPAMASHKDDEEKFFPQSGVLIFGKQGSEKTNVDIEKVMITD
EEEIRTTNPVATEQYGSVSTNLQRGNRQAATADVNTQGVLPGMVWQDRDVY
LQGPIWAKIPHTDGHFHPSPLMGGFGLKHPPPQILIKNTPVPANPSTTFSA
AKFASFITQYSTGQVSVEIEWELQKENSKRWNPEIQYTSNYNKSVNVDFTV
DTNGVYSEPRPIGTRYLTRNL

The protein sequence may be substantially similar to SEQ ID NO:08, where by substantially similar is meant that sequence identity is 60% or greater, such as 75% or greater and including 80, 85, 90, or even 95% or greater.

In some cases, the nucleic acid sequence is
SEQ ID NO: 09:
ATGGCTGCCGATGGTTATCTTCCAGATTGGCTCGAGGACACTCTCTCTGAA
GGAATAAGACAGTGGTGGAAGCTCAAACCTGGCCCACCACCACCAAAGCCC
GCAGAGCGGCATAAGGACGACAGCAGGGGTCTTGTGCTTCCTGGGTACAAG
TACCTCGGACCCTTCAACGGACTCGACAAGGGAGAGCCGGTCAACGAGGCA
GACGCCGCGGCCCTCGAGCACGACAAAGCCTACGACCGGCAGCTCGACAGC
GGAGACAACCCGTACCTCAAGTACAACCACGCCGACGCGGAGTTTCAGGAG
CGCCTTAAAGAAGATACGTCTTTTGGGGGCAACCTCGGACGAGCAGTCTTC
CAGGCGAAAAAGAGGGTTCTTGAACCTCTGGGCCTGGTTGAGGAACCTGTT
AAGACGGCTCCGGGAAAAAAGAGGCCGGTAGAGCACTCTCCTGTGGAGCCA
GACTCCTCCTCGGGAACCGGAAAGGCGGGCCAGCAGCCTGCAAGAAAAAGA
TTGAATTTTGGTCAGACTGGAGACGCAGACTCAGTACCTGACCCCCAGCCT
CTCGGACAGCCACCAGCAGCCCCCTCTGGTCTGGGAACTAATACGATGGCT
ACAGGCAGTGGCGCACCAATGGCAGACAATAACGAGGGCGCCGACGGAGTG
GGTAATTCCTCGGGAAATTGGCATTGCGATTCCACATGGATGGGCGACAGA
GTCATCACCACCAGCACCCGAACCTGGGCCCTGCCCACCTACAACAACCAC
CTCTACAAACAAATTTCCAGCGCTTCAACGGGAGCCTCGAACGACAATCAC
TACTTTGGCTACAGCACCCCTTGGGGGTATTTTGACTTCAACAGATTCCAC
TGCCACTTTTCACCACGTGACTGGCAAAGACTCATCAACAACAACTGGGGA
TTCCGACCCAAGAGACTCAACTTCAAGCTCTTTAACATTCAAGTCAAAGAG
GTCACGCAGAATGACGGTACGACGACGATTGCCAATAACCTTACCAGCACG
GTTCAGGTGTTTACTGACTCGGAGTACCAGCTCCCGTACGTCCTCGGCTCG
GCGCATCAAGGATGCCTCCCGCCGTTCCCAGCAGACGTCTTCATGGTGCCA
CAGTATGGATACCTCACCCTGAACAACGGGAGTCAGGCAGTAGGACGCTCT
TCATTTTACTGCCTGGAGTACTTTCCTTCTCAGATGCTGCGTACCGGAAAC
AACTTTACCTTCAGCTACACTTTTGAGGACGTTCCTTTCCACAGCAGCTAC
GCTCACAGCCAGAGTCTGGACCGTCTCATGAATCCTCTCATCGACCAGTAC
CTGTATTACTTGAGCAGAACAAACACTCCAAGTGGAACCACCACGCAGTCA
AGGCTTCAGTTTTCTCAGGCCGGAGCGAGTGACATTCGGGACCAGTCTAGG
AACTGGCTTCCTGGACCCTGTTACCGCCAGCAGCGAGTATCAAAGACATCT
GCGGATAACAACAACAGTGAATACTCGTGGACTGGAGCTACCAAGTACCAC
CTCAATGGCAGAGACTCTCTGGTGAATCCGGGCCCGGCCATGGCAAGCCAC
AAGGACGATGAAGAAAAGTTTTTTCCTCAGAGCGGGGTTCTCATCTTTGGG
AAGCAAGGCTCAGAGAAAACAAATGTGGACATTGAAAAGGTCATGATTACA
GACGAAGAGGAAATCAGGACAACCAATCCCGTGGCTACGGAGCAGTATGGT
TCTGTATCTACCAACCTCCAGAGAGGCAACAGACAAGCAGCTACCGCAGAT
GTCAACACACAAGGCGTTCTTCCAGGCATGGTCTGGCAGGACAGAGATGTG
TACCTTCAGGGGCCCATCTGGGCAAAGATTCCACACACGGACGGACATTTT
CACCCCTCTCCCCTCATGGGTGGATTCGGACTTAAACACCCTCCTCCACAG
ATTCTCATCAAGAACACCCCGGTACCTGCGAATCCTTCGACCACCTTCAGT
GCGGCAAAGTTTGCTTCCTTCATCACACAGTACTCCACGGGACAGGTCAGC
GTGGAGATCGAGTGGGAGCTGCAGAAGGAAAACAGCAAACGCTGGAATCCC
GAAATTCAGTACACTTCCAACTACGCCAAGTCTGCCAATGTGGACTTTACT
GTGGACAATAATGGCGTGTATTCAGAGCCTCGCCCCATTGGCACCAGATAC
CTGACTCGTAATCTGTAA

or substantially similar thereto, where by substantially similar is meant that sequence identity is 60% or greater, such as 75% or greater and including 80, 85, 90, or even 95% or greater.

The protein sequence encoded thereby is
SEQ ID NO. 10:
MAADGYLPDWLEDTLSEGIRQWWKLKPGPPPPKPAERHKDDSRGLVLPGYK
YLGPFNGLDKGEPVNEADAAALEHDKAYDRQLDSGDNPYLKYNHADAEFQE
RLKEDTSFGGNLGRAVFQAKKRVLEPLGLVEEPVKTAPGKKRPVEHSPVEP
DSSSGTGKAGQQPARKRLNFGQTGDADSVPDPQPLGQPPAAPSGLGTNTMA
TGSGAPMADNNEGADGVGNSSGNWHCDSTWMGDRVITTSTRTWALPTYNNH
LYKQISSASTGASNDNHYFGYSTPWGYFDENREHCHFSPRDWQRLINNNWG
FRPKRLNEKLENIQVKEVTQNDGTTTIANNLTSTVQVFTDSEYQLPYVLGS
AHQGCLPPFPADVFMVPQYGYLTLNNGSQAVGRSSFYCLEYFPSQMLRTGN
NFTFSYTFEDVPFHSSYAHSQSLDRLMNPLIDQYLYYLSRTNTPSGTTTQS
RLQFSQAGASDIRDQSRNWLPGPCYRQQRVSKTSADNNNSEYSWTGATKYH
LNGRDSLVNPGPAMASHKDDEEKFFPQSGVLIFGKQGSEKTNVDIEKVMIT
DEEEIRTTNPVATEQYGSVSTNLQRGNRQAATADVNTQGVLPGMVWQDRDV
YLQGPIWAKIPHTDGHFHPSPLMGGFGLKHPPPQILIKNTPVPANPSTTFS
AAKFASFITQYSTGQVSVEIEWELQKENSKRWNPEIQYTSNYAKSANVDFT
VDNNGVYSEPRPIGTRYLTRNL

The protein sequence may be substantially similar to SEQ ID NO:10, where by substantially similar is meant that sequence identity is 60% or greater, such as 75% or greater and including 80, 85, 90, or even 95% or greater.

Promoters useful in an AAV delivered coding sequence may include, for example constitutively active promoters, such as CMV promoters, β-actin promoters, SV-40 promoters such as 4×GRM6-SV40, etc. Commonly used ubiquitous promoters have been immediate-early cytomegalovirus (CMV) enhancer-promoter and the CAG promoter, which combines the CMV enhancer with the chicken β-actin (CBA) promoter. Promoters having more cell-type specific expression patterns may include, without limitation the regulatory region of the gamma-synuclein gene (SNCG), Nefh promoter, Mcp-1 promoter, etc.

Further details regarding viral vectors that may be employed in embodiments of the invention may be found in U.S. Pat. No. 10,004,788; the disclosure of which is herein incorporated by reference.

The nucleic acid coding sequence may be administered using a non-viral vector, for example, as a nucleic acid-liposome complex formulation. Such complexes comprise a mixture of lipids which bind to genetic material (DNA or RNA), providing a hydrophobic coat which allows the genetic material to be delivered into cells. Liposomes which can be used in accordance with the invention include DOPE (dioleyl phosphatidyl ethanol amine), CUDMEDA (N-(5-cholestrum-3-beta-ol 3-urethanyl)-N′,N′-dimethylethylene diamine). When the DNA of interest is introduced using a liposome, in some instances one first determines in vitro the optimal values for the DNA: lipid ratios and the absolute concentrations of DNA and lipid as a function of cell death and transformation efficiency for the particular type of cell to be transformed. These values can then be used in or extrapolated for use in in vivo transformation. The in vitro determinations of these values can be readily carried out using techniques which are well known in the art.

Other non-viral vectors may also be used in accordance with the present invention. These include chemical formulations of nucleic acids coupled to a carrier molecule (e.g., an antibody or a receptor ligand) which facilitates delivery to host cells for the purpose of altering the biological properties of the host cells. By the term “chemical formulations” is meant modifications of nucleic acids to allow coupling of the nucleic acid compounds to a carrier molecule such as a protein or lipid, or derivative thereof. Exemplary protein carrier molecules include antibodies specific to the cells of a targeted secretory gland or receptor ligands, i.e., molecules capable of interacting with receptors associated with a cell of a targeted secretory gland.

In some instances, the dosage that includes the IL-1Ra encoding nucleic acid also includes a suitable delivery vehicle (i.e., carrier), such as an aqueous delivery vehicle. Delivery vehicles of interest include sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as delivery vehicles. Non-limiting examples of pharmaceutically acceptable delivery vehicles include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropyl-methylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of delivery vehicles include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of delivery vehicles that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. Such compositions may further optionally include a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

A given dosage employed in methods of the invention may be a formulation containing the encoding nucleic acid, e.g., present in vector such as described above, as well as one or more excipients, carriers, stabilizers or bulking agents, which is suitable for administration to a human patient to achieve a desired diagnostic result or therapeutic or prophylactic effect. For storage stability and convenience of handling, a pharmaceutical composition can be formulated as a lyophilized (i.e. freeze dried) or vacuum dried powder, e.g., the form of a pre-unit dose, which can be reconstituted with saline or water prior to administration to a patient. Alternately, the pharmaceutical composition can be formulated as an aqueous solution. A pharmaceutical composition can contain a proteinaceous active ingredient. Unfortunately, proteins can be very difficult to stabilize, resulting in loss of protein and/or loss of protein activity during the formulation, reconstitution (if required) and during the storage prior to use of a protein containing pharmaceutical composition. Stability problems can occur because of protein denaturation, degradation, dimerization, and/or polymerization. Various excipients, such as albumin and gelatin have been used with differing degrees of success to try and stabilize a protein active ingredient present in a pharmaceutical composition. Additionally, cryoprotectants such as alcohols have been used to reduce protein denaturation under the freezing conditions of lyophilization.

Pharmaceutical compositions suitable for internal use include sterile aqueous solutions or dispersions and sterile powders for the extemporaneous preparation of sterile injectable solutions or dispersion. For intravenous or intra-articular administration, suitable carriers include physiological saline, bacteriostatic water, or phosphate buffered saline (PBS). In all cases, the composition must be sterile and should be fluid to the extent that easy syringe-ability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants such as polysorbates (Tween™) sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100™), N, N-dimethyldodecylamine-N-oxide, hexadecyltrimethylammonium bromide (HTAB), polyoxyl 10 lauryl ether, Brij 721™, bile salts (sodium deoxycholate, sodium cholate), pluronic acids (F-68, F-127), polyoxyl castor oil (Cremophor™) nonylphenol ethoxylate (Tergitol™), cyclodextrins and, ethylbenzethonium chloride (Hyamine™). Prevention of the action of microorganisms can be achieved by various antibacterial and antifungal agents, for example, parabens, chlorobutanol, phenol, ascorbic acid, thimerosal, and the like. In some instances, isotonic agents are included, for example, sugars, polyalcohols such as manitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof

The employed dosage may be provided in dosage unit form for ease of administration and uniformity of dosage. Dosage unit form as used herein refers to physically discrete units suited as unitary dosages for the human subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals. The dosages can be included in a container, pack, or dispenser together with instructions for administration.

In some embodiments, the dosage includes an AAV vector in an aqueous delivery vehicle. In addition to water (such as water for injection), in some instances, the aqueous vehicle includes a buffer, such as but not limited to: citrate buffer, phosphate buffer, acetate buffer, and bicarbonate buffer. In some instances, the delivery vehicle includes a phosphate buffer. When present, the buffer may be provided in any suitable concentration. In some instances, the aqueous delivery vehicle includes a salt, such as but not limited to sodium chloride, potassium chloride, and the like, where in some instances the salt is sodium chloride (NaCl). When present, the salt may be provided in any suitable concentration, wherein in some instances the salt concentration ranges from 100 to 200 mM, such as 125 to 175 mM, e.g., about 150 mM. In some instances, the aqueous delivery vehicle comprises a polyol. Polyols of interest include, but are not limited to: lactose, dextrose, sucrose, sorbitol, mannitol, and the like, where in some embodiments the aqueous delivery vehicle includes sorbitol. While the amount of the polyol may vary, in some instances the amount ranges from 0.1% to 10%, such as 1% to 5%. In some instances, the aqueous delivery vehicle includes a surfactant. Surfactants of interest include, but are not limited to, polymers, such as block copolymers, e.g., polyalkylene glycols, such as poloxamers. Examples of suitable surfactants include, for example, Tergitol® and Triton® surfactants (Union Carbide Chemicals and Plastics, Danbury, Conn.), Pluronic surfactants (BASF) polyoxyethylenesorbitans, for example, TWEEN® surfactants (Atlas Chemical Industries, Wilmington, Del.), polyoxyethylene ethers, for example Brij, pharmaceutically acceptable fatty acid esters, for example, lauryl sulfate and salts thereof (SDS), and like materials. While the amount of the surfactant may vary, in some instances the amount ranges from 0.0001% to 0.1%, such as 0.001% to 0.01%, and in some instances is about 0.001%.

In practicing methods of the invention, the volume of the dosage that is intra-articularly administered to the human may vary. In some embodiments, the dosage has a volume ranging from 0.25 to 25 ml, such as 0.5 to 15 ml, and including from 2 to 12 ml, where in some instances the amount ranges from 0.5 ml to 1 ml, 1 to 2 ml, 2 to 5 ml or 9 to 11 ml.

In some instances, the TTG indicated treatment regimen that is administered to the subject if the subject carries a TTG IL1RN rs419598/IL1RN rs315952/IL1RN rs9005 haplotype is a treatment regimen that comprises a disease modifying osteoarthritis drug (DMOAD) therapy. Examples of DMOAD therapies include, but are not limited to: calcitonin, bisphosphonates, strontium ranelate, iNOS inhibitors, MMP-13 inhibitors, aggrecanse inhibitors, doxycycline, cathepsin K inhibitors, BMP-7, TIMP-3, and FGF-18 and the like. DMOAD therapy may also include an IL-1 inhibitor, including antibodies or soluble receptors blocking IL-1 or IL-1 receptor, and the IL-1 receptor antagonist IL-1Ra. DMOAD therapy may also include a gene therapy vector, such as adeno-associated vectors, encoding an IL-1 inhibitor, including antibodies or soluble receptors blocking IL-1 or IL-1 receptor, and the IL-1 receptor antagonist IL-1Ra.

Where the disease condition is RA, the TTG indicated treatment regimen may be a DMARD treatment regimen. DMARDs that may be employed in such instances may encompass both small molecule drugs and biological agents. DMARDs may be chemically synthesized or may be produced through genetic engineering processes (e.g., recombinant techniques). Chemically synthesized DMARDs encompassed herein include, without limitation, azathioprine, cyclosporine (ciclosporin, cyclosporine A), D-penicillamine, gold salts (e.g., auranofin, Na-aurothiomalate (Myocrism), chloroquine, hydroxychloroquine, leflunomide, methotrexate, minocycline, sulphasalazine (sulfasalazine), and cyclophosphamide. Biological DMARDs include, without limitation, TNF-a blockers such as etanercept (Enbrel®), infliximab (Remicade®), adalimumab (Humira®), certolizamab pego (Cimzia™) and golumumab (Simponi™), IL-1 blockers such as anakinra (Kineret®.); monoclonal antibodies against B cells including rituximab (Rituxan®); T cell costimulation blockers such as abatacept (Orencia®), and IL-6 blockers such as tocilizumab (RoActemra®, Actemra®); a calcineurin inhibitor such as tacrolimus (Prograf®).

As summarized above, aspects of the methods include treating the subject based on their carriage of a rs419598/lrs315952/rs9005 haplotype. In some embodiments, a TTG indicated treatment regimen is not administered to the subject if the subject lacks a TTG haplotype and/or carries a CTA rs419598/rs315952/rs9005 haplotype, such as a CTA-1 or CTA-2 haplotype. As such, if the subject lacks a TTG haplotype, a therapy other than a TTG indicated treatment regimen may be administered. In some instances, even if the subject is TTG1, if the subject is also CTA1, the TTG indicated treatment regimen is not administered to the subject. In such instances, another treatment regimen may be administered to the subject.

The other, non TTG indicated treatment regimen that is administered to the subject may vary. In some such instances, the therapeutic regimen comprises a conservative therapy, such as but not limited to activity modification, weight loss, physical therapy, steroid therapy, non-steroidal anti-inflammatory therapy, injection therapy, and combinations thereof. In some such instances, the therapeutic regimen comprises an osteoarthritis therapy such as but not limited to acetaminophen therapy, non-steroidal anti-inflammatory drug therapy, opiate therapy, glucocorticoid therapy, hyaluronic acid therapy, stem cell therapy, autologous blood product therapy, and combinations thereof.

Subjects As summarized above, the subject is one suffering from or may be predicted to suffer from arthritis, e.g., OA or RA. Where the disease condition is OA, the particular OA may vary, wherein some instances the OA is selected from the group consisting OA of the hand, knee, hip, shoulder, ankle, elbow, temperomandibular joint, and spine, and combinations thereof. In some instances, the OA is OA of the knee. In some instances, the OA is early OA, moderate OA or severe OA.

In some instances, the subject has been diagnosed as having arthritis, e.g., OA or RA, where in some instances the method includes diagnosing the subject as having arthritis, e.g., OA or RA. The arthritis, e.g., OA or RA, of the subject may have been assessed or evaluated using one or more protocols.

In some instances where the arthritis is OA, the OA has been assessed at least in part using an imaging protocol. A variety of different imaging protocols may be employed in assessing OA. In some instances, a radiographic (i.e., X-ray) protocol is employed. For example, the structural progression of OA may be assessed on plain radiographic views by measuring the joint space width (JSW) and/or joint space narrowing (JSN) over a period of time. (Altman et al.: Osteoarthritis Cartilage 1996, 4:217-243.) OA progression is associated with accelerated cartilage degradation leading to JSN, painful joint disruption, and functional compromise. OA disease progression may be measured on a Kellgren-Lawrence Grading Scale (“KL scale”) to measure occurrence and severity of OA in human subjects. Grade 0 means the joints of a human subject is normal. Grade 1 means a human subject has doubtful narrowing of joint space and possible osteophytic lipping. Grade 2 means a human subject has definite osteophytes, definite narrowing of joint space. Grade 3 means a human subject has moderate multiple osteophytes, definite narrowing of joint space, some sclerosis and possible deformity of bone contour. Grade 4 means a human subject has large osteophytes, marked narrowing of joint space, severe sclerosis and definite deformity of bone contour. In some instances, the OA of the human has been assigned a Kellgren-Lawrence score of 2, 3 or 4. In some instances, the human has been assigned a Kellgren-Lawrence score of 2 or 3.

In some instances, the OA is assessed using a magnetic resonance imaging (MRI) protocol, e.g., to identify bone marrow lesions. Bone marrow lesions (or edemas) are very strongly associated with knee arthritis pain (Felson et al., “The association of bone marrow lesions with pain in knee osteoarthritis.” Ann Intern Med. 2001 Apr. 3; 134(7):541-9) and disease progression (Felson et al., “Bone marrow edema and its relation to progression of knee osteoarthritis.” Ann Intern Med. 2003 Sep. 2; 139(5 Pt 1):330-6). See also U.S. Pat. No. 6,564,083 to Stevens, which describes a method of identifying in a patient having joint pain the susceptibility of the patient to developing progressive OA or loss of joint space, by determining the presence or absence of bone marrow edema about or of the joint. The determination may be made through the use of MRI. See also U.S. Published Patent Application 20170202520 the disclosure of which is herein incorporated by reference.

In some instances, the OA has been assessed using a subjective protocol, such as a pain reporting protocol. In some instances a pain assessment scale such as the Visual Analog Scale (“VAS”) is employed, where a patient is asked to indicate a point on a 100 millimeter line, having a left anchor of no pain and a right anchor of worst possible pain, corresponding to their degree of pain or the Likert score, wherein a patient is asked to categorize their pain on a numerical scale of from 0 (no pain) to 10 (worst possible pain). Additional subjective approaches that may be employed in assessing the OA include, but are not limited to: the Western Ontario and MacMaster Universities Osteoarthritis Index (WOMAC) pain questionnaire; the McGill Pain Questionnaire; the 36-Item Short Form Health Survey (SF-36); and the like.

In some embodiments, the OA has been assessed at least in part using a biomarker evaluation protocol. Such assessment may include measuring, observing or detecting the presence or activity in a biomarker. In various embodiments, the biomarker is a molecule that indicates presence or extent of the pain in the individual. The assessment may include measuring, observing or detecting a change in concentration or activity of the biomarker over a period of time may be employed. For example, the assessment may include observing a reduction in amount of the biomarker. Alternatively, the change is an increase in amount of the biomarker. In some instances, the measuring, observing or detecting includes using an assay, a questionnaire, a strip, a well, a gel, a detector, an indicator, a dye, an imager, and a slide.

In various embodiments, the biomarker includes at least one compound selected from the group consisting of: a carbohydrate; a peptide; a protein; and a genetic material, e.g., DNA or RNA (collectively referred to as “nucleic acid”). In various embodiments of the method, the biomarker includes a growth factor, an interleukin (IL), an osteoinductive factor, an interferon, a tumor necrosis factor (TNF), a steroid, a proteoglycan, a collagen or collagen fragment, a fiber, a serum protein, an immunoglobulin, a hormone.

In various embodiments of the method, the biomarker includes at least one selected from the group consisting of: a cell; a peptide or protein expressed by the cell; or a molecule that binds to the cell. For example, the biomarker is located in the serum or the cartilage of the individual. In various embodiments of the method, the biomarker comprises at least one selected from the group consisting of: a high-sensitivity C-reactive protein (hsCRP); a matrix metallopeptidase (MMP; for example MMP-9); a vascular endothelial growth factor (VEGF), a MMP degradation product for example a MMP degradation product of type I, II, or III collagen (C1M, C2M, C3M); a C-reactive protein (CRPM), CTX-1, CTX-II, TIINE, creatinine, and a vimentin (for example a citrullinated and MMP-degraded vimentin, VICM). In some instances, the biomarker evaluation protocol comprises assaying a biomarker selected from the group consisting of IL-1, TNF-α, IL-1Ra, COMP, CTXII, sGAG, NTX-1, MMP1, MMP3 and MMP9, and combinations thereof.

Biomarkers of interest include, but are not limited to: sIL-6R, IL-6, hsCRP and the like. With respect to sIL-6R, decreased levels of this biomarker relative to those observed in a healthy control may be employed to infer the presence of a TTG haplotype. With respect to IL-6, increased levels of this biomarker relative to those observed in a healthy control may be employed to infer the presence of a TTG haplotype. With respect to hsCRP, increased levels of this biomarker relative to those observed in a healthy control may be employed to infer the presence of a TTG haplotype. In making an inference of the presence of a TTG haplotype, one, two or all of these biomarkers may be employed, where these biomarkers may be employed in combination with IL-1Ra, e.g., as described above. The biomarkers may be assayed using any convenient protocols, such as those described above in connection with IL-1Ra, as well as below. With respect to RA, the above markers and/or TTG haplotype determined using another protocol, such as described above, may be employed to infer increased disease activity (DAS28).

Measuring, observing or detecting the biomarker in various embodiments comprises obtaining a sample from the individual. For example, the sample is selected from: a cell, a fluid, and a tissue. In various embodiments of the method, the fluid is at least one selected from the group consisting of: serum, plasma, synovial fluid, saliva, and urine. In various embodiments of the method, the cell or the tissue is at least one selected from the group consisting of: vascular; epithelial; endothelial; dermal; connective; muscular; neuronal; soft tissue including cartilage and collagen; bone; bone marrow; joint tissue; and an articular joint. For example, the sample is collected after administering the binding protein and the biomarker is measured, observed or detected. These biomarker data are then compared to a suitable control sample or predetermined standard.

In any of the above methods, the biological sample or samples may comprise any one of synovial fluid, whole blood, blood plasma, serum, urine, and saliva. In some instances, the protein level determination assay one or more polypeptides is employed. In some embodiments, the protein level is measured using a method selected from the group consisting of: LUMINEX, ELISA, immunoassay, mass spectrometry, high performance liquid chromatography, two-dimensional electrophoresis, Western blotting, protein microarray, and antibody microarray.

In other embodiments, a nucleic acid expression assay may be employed. The expression levels (or expression profile) can be then determined using any convenient method, where such methods include, but are not limited to, polymerase-based assays such as qPCR, RT-PCR (e.g., TAQMAN or SYBR Green), hybridization-based assays such as DNA microarray analysis, flap-endonuclease-based assays (e.g., INVADER), and direct mRNA capture (QUANTIGENE or HYBRID CAPTURE (Digene)). See, for example, US 2010/0190173 for descriptions of representative methods that can be used to determine expression levels.

In some instances, peripheral blood leukocytes (PBL) are employed as a source of nucleic acid to be used in assays. PBLs can be obtained from an individual in the form of a Peripheral Blood Mononuclear Cell (PBMC) sample. PBMCs are a mixture of monocytes and lymphocytes, and there are a number of known methods for isolating PBMCs from whole blood. While any suitable method may be employed, in one embodiment, PBMCs are isolated from whole blood samples using density gradient centrifugation. Alternatively, PBL may be further isolated from whole blood or PBMCs to yield a cell subpopulation, such as a population of lymphocytes (e.g., T-lymphocytes or sub-population thereof). Examples for isolating such sub-populations include cell sorting or cell-capturing using antibodies to particular cell-specific markers. In another embodiment, PBL can be obtained from whole blood using the PAXgene kit (Qiagen).

RNA can be extracted from the collected cells (e.g., from PBMC or PBL samples or from blood plasma) using any convenient protocol. For example, RNA may be purified from cells using a variety of standard procedures as described, for example, in RNA Methodologies, A laboratory guide for isolation and characterization, 2nd edition, 1998, Robert E. Farrell, Jr., Ed., Academic Press. In addition, various commercial products are available for RNA isolation. As would be understood by those skilled in the art, total RNA or polyA+RNA may be used for preparing gene expression profiles.

In some instances, the subject has persistently suffered from one or more symptoms of OA despite undertaking a non-steroidal anti-inflammatory drug (NSAID) treatment regimen. A NSAID treatment regimen is a treatment regimen that includes administration of one or more NSAIDs, where examples of NSAIDs include, but are not limited to: acetaminophen (Tylenol), Aspirin (brand names include Bayer, Bufferin, and Ecotrin, St. Joseph); Ibuprofen (Advil, Motrin); Naproxen (Aleve, Anaprox DS, Naprosyn); Celecoxib (Celebrex); and the like. In yet other embodiments, the human that is treated according to embodiments of the invention is one that has undergone or is undergoing an NSAID treatment regimen, e.g., where the subject has been administered an NSAID treatment regimen for 1 to 60 months.

In some embodiments, the subject is one that has failed a three-month trial of a minimum of two conservative therapies for the OA. Conservative therapies included, but are not limited to: activity modification, weight loss, physical therapy, steroid therapy, non-steroidal anti-inflammatory therapy, injection therapy (such as hyaluronic acid, steroids, Zilretta, and the like), and combinations thereof.

In some instances, the subject is one that is not undergoing anti-rheumatic disease medication therapy. Anti-rheumatic disease medication therapy is a treatment regimen that includes administration of a Disease-modifying antirheumatic drug (DMARD). DMARD is a category of otherwise unrelated drugs defined by their use in rheumatoid arthritis to slow down disease progression. DMARDs include, but are not limited to: Abatacept, adalimumab, anakinra, azathioprine, chloroquine (anti-malarial), ciclosporin (Cyclosporin A), D-penicillamine, etanercept, golimumab, gold salts (sodium aurothiomalate, auranofin), hydroxychloroquine, infliximab, leflunomide, methotrexate (MTX), minocycline, rituximab, sulfasalazine (SSZ), tocilizumab, tofacitinib, and the like.

In various embodiments, the method further comprises observing a reduction in an indicium of the arthritis, e.g., OA. In various embodiments, the method further comprises observing a reduction in a condition associated with the arthritis, e.g., OA. For example, where the disease is OA, the indicium or condition is presence of an osteophyte, bone sclerosis, effusion, joint swelling, synovitis, synovial hypertrophy and hyperplasia, angiogenesis, inflammation, stiffness, joint space narrowing, or pain associated with the OA.

In various embodiments, the method further includes observing or detecting a modulation (e.g., reduction or increase) in presence or activity of a biomarker. In various embodiments, the biomarker indicates presence or extent of the arthritis, e.g., OA or RA. For example, the biomarker corresponds to presence of inflammation. In various embodiments of the method, the biomarker comprises at least one selected from the group consisting of: a carbohydrate; a peptide; a protein; and a genetic material. For example, the genetic material comprises DNA or RNA.

The biomarker comprises in various embodiments at least one selected from the group consisting of: a cell; a peptide or protein expressed by the cell; or a molecule that binds to the cell. In various embodiments of the method, the biomarker comprises a monocyte, a macrophage, B cells, T cells, a cytokine, (e.g., TNF, and IL-1Ra), a growth factor, an interleukin (e.g., IL-4, IL-6, IL-10, and IL-13), an osteoinductive factor, an interferon, a tumor necrosis factor, a steroid, a proteoglycan, a fiber, a collagen or collagen fragment, a serum protein, an immunoglobulin, or a hormone. In various embodiments of the method, the biomarker comprises at least one selected from the group consisting of: C-reactive protein (CRP); a matrix metallopeptidase (MMP; for example MMP-9); a vascular endothelial growth factor (VEGF), a MMP degradation product for example MMP degradation product of type I, II, or III collagen (C1M, C2M, C3M); a prostaglandin, nitric oxide, a disintegrin and metalloproteinase with thrombospondin motifs (ADAMTS), an adipokine, an endothelial growth factor (EGF), a bone morphogenetic protein (BMP), a nerve growth factor (NGF), a substance P, an inducible nitric oxide synthase (iNOS), CTX-1, CTX-II, TIINE, creatinine, and a vimentin (for example a citrullinated and MMP-degraded vimentin; VICM). In various embodiments, the biomarker comprises a local tissue degradation biomarker.

In various embodiments herein, observing or detecting the biomarker comprises obtaining a sample from the individual. In various embodiments, the sample is selected from: a cell, a fluid, and a tissue. For example, the fluid is at least one selected from: serum, plasma, synovial fluid, saliva, and urine. The cell or the tissue comprises for example at least one type selected from: vascular; epithelial; endothelial; dermal; connective; muscular; neuronal; soft tissue for example cartilage, synovium, capsule and collagen; bone; bone marrow; joint tissue; and an articular joint. For example, the biomarker is detected using an assay, a computer, or a probe. For example, the probe is a molecular probe that detects the presence of the biomarker. In an embodiment, the binding protein reduces the OA and/or modulates (e.g., reduces and increases) expression and/or activity of the biomarker by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments of the method, the method reduces the OA in at least one metric or criteria from the group consisting of: Western Ontario and McMaster Universities Arthritis Index (WOMAC), The Knee injury and Osteoarthritis Outcome Score (KOOS), International Knee Documentation Committee (IKDC) score, Whole-Organ Magnetic Imaging Score (WORMS), Intermittent and Constant Osteoarthritis Pain (ICOAP) score; 11-point Numeric Rating Score (NRS) score, and the individual's assessment (for example a questionnaire or a patient's global assessment). In various embodiments, observing or evaluating is performed over a period of time selected from the group consisting of: hours, days, weeks, and months. In various embodiments, observing or evaluating determines that the method does not produce adverse effects in the individual. In various embodiments of the method, observing or evaluating determines that the method is at least one characteristic selected from the group consisting of: efficacious, therapeutic, safe, and producing beneficial biochemical and/or effects in the individual. In an embodiment, the method reduces the OA and/or modulates the metric by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

The method in various embodiments further comprises observing or detecting a reduction in an indicium of the pain. For example, the pain condition is associated with knee OA or erosive hand OA. In various embodiments of the method, the pain is nociceptive pain associated with OA. For example, the pain is mechanical nociceptive pain associated with OA.

In some instances, embodiments of the method result in persistent amelioration of one or more symptoms of the arthritis, e.g., OA or RA, such as pain. In some instances, the method results in a reduction in pain, e.g., as determined using one or more above described assessment protocols, such as a visual analog scale. Where reduction in pain is determined using a visual analog scale, the magnitude of pain reduction is manifested by a movement along of the scale of 10% or more of the length of scale, such as 20% or more of the length of scale, including 30% or more of the length of scale, as well as 40% or more, including 50% or more, of the length of scale. While the duration of symptom, e.g., pain, amelioration may vary, in some instances the persistent amelioration lasts for 3 months or longer, such as 6 months or longer, including 9 months or longer, e.g., 12 months or longer, 18 months or longer, 24 months or longer, 30 months or longer, 36 months or longer, and in some instances the persistent amelioration lasts for 3 to 36 months.

In some instances, the method results in a synovial fluid IL-1Ra concentration ranging from 0.1 ng/ml to 400 ng/ml, such as 1 ng/ml to 400 ng/ml, such as 10 ng/ml to 100 ng/ml or 20 ng/ml to 50 ng/ml, for a period of 1 to 36 months or longer following administration, such as 1 month, 3 months, 6 months, 12 months, 18 months, 24 months, 30 months, 36 months following administration to the human. Synovial fluid IL-1Ra concentration may be determined using any convenient protocol, e.g., by taking a sample of synovial fluid and assays for IL-1Ra protein therein, e.g., using the assays described above.

In some instances, the method further results in a modification of joint structure of the human, such as reduction in tissue degeneration and/or reduction of joint space narrowing. As such, methods may result in restoration, at least to some extent, of joint structure of the human, where restoration means a return, a least in part, to the structure of a health joint of a non-osteoarthritic human. In some instances, the method results in a preservation of joint structure of the human, e.g., so that the joint structure does not further change but is stabilized. In such instances, the modification or preservation may be determined by an imaging protocol, such as the radiographic and/or magnetic resonance imaging protocols described above.

The term “subject” is used interchangeably in this disclosure with the term “patient”. In certain embodiments, a subject is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult. While the devices and methods described herein may be applied to perform a procedure on a human subject, it is to be understood that the subject devices and methods may also be carried out to perform a procedure on other subjects (that is, in “non-human subjects”). In some embodiments where the subject is a human, the human is Caucasian. In other embodiments where the subject is human, the human may be non-Caucasian, such as Asian, African-American or Latino or of Asian, African-American or Latino descent.

### Post-Bariatric Surgery

Aspects of the invention include using the above described IL1RN TTG haplotype as a biomarker to predict weight loss and/or decreased obesity-associated inflammation following bariatric surgery. In some instances, bariatric surgery patients are assayed for the presence or absence of a TTG rs419598/rs315952/rs9005 haplotype (e.g., as described above), where the particular assay method may be any convenient method, such as described above. As such, bariatric surgery patents are, in some instances, assayed to determine whether they carry a TTG rs419598/rs315952/rs9005 haplotype. Bariatric surgery patients having TTG-1 or TTG-2 haplotype are, in some instances, determined to have lower potential for weight loss following bariatric surgery, e.g., relative to those bariatric patients having a TTG-0 haplotype. Bariatric surgery patients having TTG-1 or TTG-2 haplotype are, in some instances, determined to have increased obesity-associated inflammation following bariatric surgery, e.g., relative to those bariatric patients having a TTG-0 haplotype. Other determinations for bariatric surgery patients that may be made based on whether they have a TTG haplotype are described in the Experimental Section, below. Patients evaluated in such embodiments may be patients of any bariatric surgery, including but not limited to gastric bypass, sleeve gastrectomy, adjustable gastric band, and biliopancreatic diversion with duodenal switch. In some instances, the bariatric patients may suffer from arthritis, e.g., as described above. In some instances, post-bariatric surgery therapeutic/dietary regimen may be modified based on whether the bariatric patient has a TTG, e.g., TTG-1 or TTG-2, haplotype. For example, bariatric patients having a TTG haplotype may be prescribed one or more of a lower-calorie diet, an appetite suppressant, etc.

### Kits & Systems

Also provided are kits and systems that find use in practicing embodiments of the methods, such as those described as described above. The term “system” as employed herein refers to a collection of two or more different active agents, present in a single composition or in disparate compositions, that are brought together for the purpose of practicing the subject methods. The term “kit” refers to a packaged active agent or agents.

For example, kits and systems for practicing the subject methods may include one or more reagents that find use in genotyping a subject, e.g., as described above. In addition, the kits and systems may include pharmaceutical formulations, e.g., dosages in the form of unit doses or pre-unit doses, such as described above. In certain embodiments the kits may include a single pharmaceutical composition, present as one or more unit doses. In some embodiments, the kits may include two or more separate pharmaceutical compositions, each containing a different active compound.

In certain embodiments, the kit includes packaging configured to hold kit contents, e.g., reagents and/or pharmaceutical formulations. The packaging may be a sealed packaging, e.g., a water vapor-resistant container, optionally under an air-tight and/or vacuum seal. In certain instances, the packaging is a sterile packaging, configured to maintain the device enclosed in the packaging in a sterile environment. By “sterile” is meant that there are substantially no microbes (such as fungi, bacteria, viruses, spore forms, etc.).

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, portable flash drive, etc., on which the information has been recorded. Yet another means that may be present is a website address which may be used via the internet to access the information at a removed site. Any convenient means may be present in the kits.

As can be appreciated from the disclosure provided above, embodiments of the present disclosure have a wide variety of applications. Accordingly, the examples presented herein are offered for illustration purposes and are not intended to be construed as a limitation on the embodiments of the present disclosure in any way.

### EXAMPLES

### I. Interleukin 1 Receptor Antagonist (IL1RN) Gene Variants Predict Radiographic Severity of Knee Osteoarthritis and Risk of Incident Disease

### A. ABSTRACT

1. Objective: In these studies, we examined the association of single nucleotide polymorphisms (SNPs) of the IL1RN gene with radiographic severity of symptomatic knee OA (SKOA) and the risk of incident OA. We also explored these genetic polymorphisms in patients with rheumatoid arthritis.
2. Methods: Over 1000 subjects who met ACR criteria for tibiofemoral OA were selected from three independent, NIH-funded cohorts. Haplotypes formed from three SNPs of the IL1RN gene [rs419598, rs315952, rs9005] were assessed for association with radiographic severity, and risk for incident radiographic OA (rOA) in a nested case-control cohort. This IL1RN haplotype also was assessed for association with disease activity (DAS28) and plasma inflammatory markers in patients with rheumatoid arthritis (RA).
3. Results: Carriage of the IL1RN TTG haplotype was associated with increased odds of more severe rOA compared to age-, sex- and BMI-matched individuals. Examination of the OAI Incidence Subcohort demonstrated that carriage of the TTG haplotype was associated with 4.1-fold (p=0.001) increased odds of incident rOA. Plasma IL-1Ra levels were lower in TTG carriers and chondrocytes from IL1RN TTG carriers exhibited decreased secretion of IL-1Ra. In patients with RA, the IL1RN TTG haplotype was also associated with increased DAS28, decreased plasma IL-1Ra and elevations of plasma inflammatory markers (hsCRP, IL-6).
4. Conclusion: Carriage of the IL1RN TTG haplotype is associated with more severe rOA, increased risk for incident OA and higher DAS28 in patients with RA. These changes are accompanied by decreased plasma IL-1Ra, suggesting defective endogenous “anti-inflammation” capacity in these patients.

### B. INTRODUCTION

Osteoarthritis (OA) is characterized by focal loss of joint articular cartilage, osteophyte formation, and subchondral bone remodeling. The production of interleukin 1 (IL-1p) and other mediators produced by cartilage and synovium induce a state of chronic low-grade inflammation that contributes to disease pathogenesis (1-4). Multiple genome-wide associations and candidate gene studies have identified genetic variants involved in the pathogenesis of OA (5-8), including variants in GDF5, VDR, IGF-1, COL11A1, and VEGF. However, genetic studies have not identified any compelling high-impact loci, but rather support the polygenic nature of the disease, consistent with the contribution of multiple variants with small effect sizes to variation in OA susceptibility or severity (9-11).

We have reported inflammatory gene associations with radiographic severity in two small populations of patients with symptomatic tibiofemoral knee OA recruited at the NYU Langone Orthopedic Hospital (n=80) and from the Prediction of Osteoarthritis Progression study followed at Duke University (n=50) (12). We examined 15 single nucleotide polymorphisms (SNPs) in six inflammatory response genes, including those for IL-1α, IL-1β, IL-1RN, TNFα, IL-10, estrogen receptor 1 (ESR1) and determined whether polymorphisms of these genes could stratify patients with knee OA into high and low risk for radiographic knee OA severity. We found that radiographic severity was associated only with a three SNP haplotype (rs419598, rs315952, and rs9005) of IL1RN, the product of which is IL-1Ra (12).

The goal of this study was to confirm and extend these findings in over 1000 additional individuals followed in three independent, NIH-funded, prospective cohorts of individuals with, or at risk for, knee OA. In addition, to determine whether the findings were restricted to osteoarthritis, we extended our studies to patients with rheumatoid arthritis (RA).

### C. PATIENTS AND METHODS

1. Participants with symptomatic knee OA. We assembled a total of 1066 subjects, based on comparable eligibility criteria from three independent, NIH-funded, prospective cohorts of individuals with, or at risk for, knee OA. Participants met clinical (American College of Rheumatology) and radiographic criteria for tibiofemoral OA [Kellgren-Lawrence (KL) score 1)]; all had body mass index (BMI)<33 kg/m². Exclusion criteria included histories of intra-articular corticosteroid injection within 3 months of enrollment, bilateral knee replacements, and other forms of arthritis, cancer or other chronic diseases beyond hypertension or hypercholesterolemia. Using these eligibility criteria, we established a study population by including 300-400 subjects from three independent cohorts with the goal of reducing phenotypic heterogeneity across populations that would otherwise result from differences in site-specific enrollment criteria, demographics and study design.

Patients in all three cohorts underwent standardized weight-bearing fixed-flexion postero-anterior knee radiographs with a positioning frame to control the degree of flexion and leg rotation (SynaFlexer; Synarc, San Francisco, Calif., USA). Radiographs were scored for tibiofemoral Kellgren-Lawrence (KL) grade (0-4), and minimal joint space width (mJSW) for each cohort as previously described (1, 13-15).

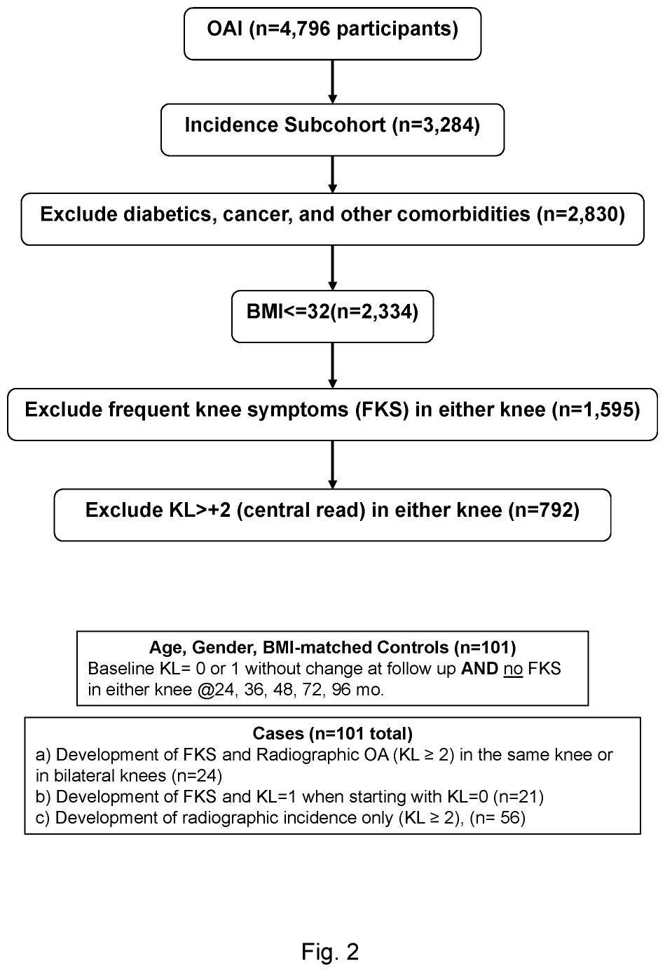

2. NYU OA Cohort. To validate our original observation linking IL1RN haplotypes to OA severity from the “founding” cohort of 80 NYU and 50 Duke SKOA patients (12, 16), we recruited and followed 372 additional SKOA patients between 2008-2016. Individuals who comprised the “founding” cohort are not included in this study.
3. Genetics of Generalized Osteoarthritis (GOGO). We applied the same inclusion/exclusion criteria to select a subset of participants in the GOGO study from Duke University (13). Review of the GOGO cohort identified 339 individuals who met the eligibility criteria and had IL1RN genotyping available for analysis. None of the GOGO patients selected for this study were among the participants included in the previously reported “founding” cohort (12).
4. Osteoarthritis Initiative (OAI). We applied identical criteria to select a corresponding subset from the Osteoarthritis Initiative (OAI). A group of 4,796 men and women between the ages of 45 and 79 years who had or were at risk for symptomatic knee OA were enrolled at 4 academic centers within the United States between February 2004 and May 2006. The OAI recruited groups of individuals divided into two sub-cohorts, those with symptomatic radiographic knee OA in at least one knee at risk of disease progression (the “Progression” sub-cohort), and those at high risk of initiation of symptomatic radiographic knee OA in one or both knees (the “Incidence” sub-cohort). Inclusion and exclusion criteria for entry into the Progression and Incidence Sub-cohorts are available at http://oai.epi-ucsf.org/datarelease/. Genomic DNA was obtained from the OAI Tissue Biorepository and x-ray image data from OAI. To assess radiographic severity, we selected 424 patients from the Progression subcohort whose knee radiographs at baseline exhibited high-quality medial tibial plateau (MTP) alignment using OAI patient identifiers provided by Dr. Eric Vignon, as described (14). Of these, 355 patients met the NYU eligibility criteria for SKOA [age >35 years; body mass index (BMI)<33, and knee pain reported in the OAI database using the Numerical Rating Scale (NRS)]. Data used in the preparation of this article were obtained from the Osteoarthritis Initiative (OAI) database, which is available for public access at http://www.oai.ucsf.edu/. Specific datasets used are [Version 25 release date Aug. 1, 2017].
5. Selection and analysis of incident OA from the OAI. For analyses of IL1RN haplotypes as predictors of incident OA, we analyzed the OAI Incidence Subcohort. We utilized a nested case-control design to study an equal number of age-, BMI-, comorbidity- and sex-matched control patients who did not develop incident OA (see FIG. 2—Incidence flow chart).

At the time of enrollment in the Incidence Subcohort subjects did not meet criteria for OA and were assessed at 24, 36, 48, 72, and 96 months. We focused on the subgroup with neither radiographic knee OA nor frequent knee pain at baseline, but with other risk factors for developing radiographic knee OA, as described (http://oai.epi-ucsf.org/datarelease/). Incident cases of symptomatic knee OA are defined in the OAI as the first occurrence during the study of frequent knee symptoms in the presence of definite tibiofemoral knee OA (by OARSI grade 1-3) in the same knee. Analysis of the OAI database indicated that 792 participants had neither radiographic evidence of knee OA nor frequent knee pain at baseline, but had other risk factors for developing radiographic knee OA. We identified 101 OAI-defined incident cases diagnosed within 2-4 years of baseline assessment based on two subgroup categories: a) symptomatic knee OA incidence and b) radiographic-only incidence, as shown in the chart. We then performed a nested case-controlled study, comparing 101 control subjects who did not meet criteria for incident OA over a minimum of 4 years and for up to 96 months of follow-up, matched for sex, age, and BMI.

6. NYU RA cohort. Plasma samples from RA subjects (N=145) were selected for analysis. The RA patients were DMARD-naïve or not currently treated with DMARDs or prednisone >5 mg per day. Patients who received intra-articular steroids or hyaluronans within 3 months were excluded. Clinical assessments included tender and swollen 28-joint counts, patient global disease activity assessment (0-100), and ESR to enable calculation of the DAS28-ESR. Informed consent was obtained for all patients with approval from the New York University institutional review board. As we have reported previously, the RF factor did not interfere in the ELISA with any of the markers studied (Jain et al., Increased plasma IL-17F levels in rheumatoid arthritis patients are responsive to methotrexate, anti-TNF, and T cell costimulatory modulation. Inflammation. 2015 February; 38 (1):180-6; Sher et al., Periodontal disease and the oral microbiota in new-onset rheumatoid arthritis. Arthritis Rheum. 2012 October; 64 (10):3083-94.).
7. IL-1RN genotyping for NYU, GOGO and OAI cohorts. For the NYU cohort (both OA and RA), genomic DNA from EDTA tubes was isolated using the QIAamp DNA Blood Mini Kit (Qiagen, USA). For the OAI cohort, we received blinded samples of purified genomic DNA from OAI biospecimen collection center. For the GOGO cohort, DNA was isolated, as reported previously (17). Genotyping for the IL-1RN SNPs (rs419598 (C_8737990_10), rs315952 (C_11512470_10), rs9005 (C_3133528_10) for NYU and OAI cohorts were accomplished by polymerase chain reaction (PCR) using validated commercial SNP primers and probes (Applied Biosystems, CA, USA) along with detection using allelic discrimination computation (ABI Prism 7900HT Detection Systems). Genotypes were analyzed using TaqMan Genotyper Software version 1.4. All genotype and association studies were performed blinded to patient clinical data.

All GOGO participants with longitudinal data were genotyped at the David H. Murdock Research Institute (Kannapolis, N.C.) using the Illumina BeadChip that consisted of 550,000 HapMap SNPs and 60,000 custom SNPs. Genotypes were called with Illumina GenomeStudio. Sample- and SNP-level QC performed as previously described (18). For the GOGO study, rs315952 and rs419598 were collected from directly genotyped data, and rs9005 was imputed with high imputation quality (INFO >0.8) as described (18).

8. Haplotype determination. Since all three SNPs (rs419598, rs315952, and rs9005) were in the IL1RN gene, we evaluated haplotype effects on radiographic severity as described in our previous publication (12). The linkage disequilibrium (LD) parameters D′ and r2 for IL1RN SNPs rs419598, rs315952, and rs9005 are shown in Table 6 for all three cohorts. Haplotypes TTG-0 or TTG-1 or TTG-2, respectively, represent carriers of 0 or 1 or 2 copies of IL1RN haplotype produced using 3 IL1RN SNPs (rs419598, rs315952 and rs9005).
9. Blood sample collection: For the NYU cohort, non-fasting blood samples were collected at baseline in pyrogen-free heparinized or EDTA tubes for isolation of plasma and genomic DNA, respectively. Plasma samples were immediately aliquoted and stored at −80° C. for future use.
10. Chondrocyte functional studies. OA cartilage was obtained from 45 separate patients undergoing total knee replacement surgery at NYU Langone Orthopedic Hospital and chondrocytes were isolated as described previously (19, 20). Chondrocytes are were grown at a density of 5×10⁵ cells/well in 6-well plates. The cells were adapted to serum-free media overnight and stimulated with IL-1β(10 ng/ml) for 24 h. Total protein was extracted as described previously (19, 20) and supernatants collected for determination of inflammatory mediators. IL1RN genotyping was performed as described above using the TaqMan allelic discrimination assay.
11. Cytokine and IL-1Ra Measurements. Plasma, chondrocyte culture supernatants, and total cell lysates were analyzed for IL-1Ra levels using IL-1Ra kits (Cat #DRAOOB), IL-6 (#HS600C), soluble IL-6 receptor alpha (#DR600); R&D systems, Minneapolis, Minn., USA).and high sensitive CRP (hsCRP #07BC-1119; MP Biomedicals) as described previously (Bournazou et al., Vascular Adhesion Protein-1 (VAP-1) as Predictor of Radiographic Severity in Symptomatic Knee Osteoarthritis in the New York University Cohort. Int J Mol Sci. 2019 May 29; 20(11).
12. Statistical analyses. IL1RN haplotype frequencies were calculated in SKOA patients in all three cohorts. Three IL1RN SNPs, previously implicated in risk of knee OA, rs419598, rs315952, and rs9005 were tested for association with knee OA severity. Haplotypes TTG-0 or TTG-1 or TTG-2, respectively, represent carriers of 0 or 1 or 2 copies of IL1RN haplotype produced using 3 IL1RN SNPs (rs419598, rs315952 and rs9005). Primary analyses evaluated associations between haplotypes and radiographic severity. To be consistent with our previously published work, we classified patients as severe knee OA if the KL score was 3 or 4 and as mild-to-moderate knee OA if the KL score was 1 or 2. Genotype associations with radiographic severity were determined using Fisher's exact test, adjusted using the false discovery rate (FDR), where appropriate.

For a continuous trait outcome, mJSW versus age, we used a regression model. Age and mJSW correlation were plotted, and at each age interval, the expectation of mJSW was calculated with a 95% confidence interval. The difference of mJSW for each haplotype group within each age interval was evaluated using Student's t-test. For each cohort, we also fit a linear regression of the mJSW against IL1RN haplotypes, beta coefficients, and p-values for IL1RN haplotypes were reported. For this process, we also performed separate modeling after conditioning out effects of age/sex/BMI/by adding age/sex/BMI/cohort as covariates.

13. Ethics approval and consent to participate. The current study was performed in accordance with the ethical standards of the Helsinki Declaration of 1975, as revised in 2000, and studies (#i9018 and 12-03682) were approved by the Institutional Review Board (IRB) of NYU School of Medicine. All patients provided written, informed consent to participate in the study.

### D. RESULTS

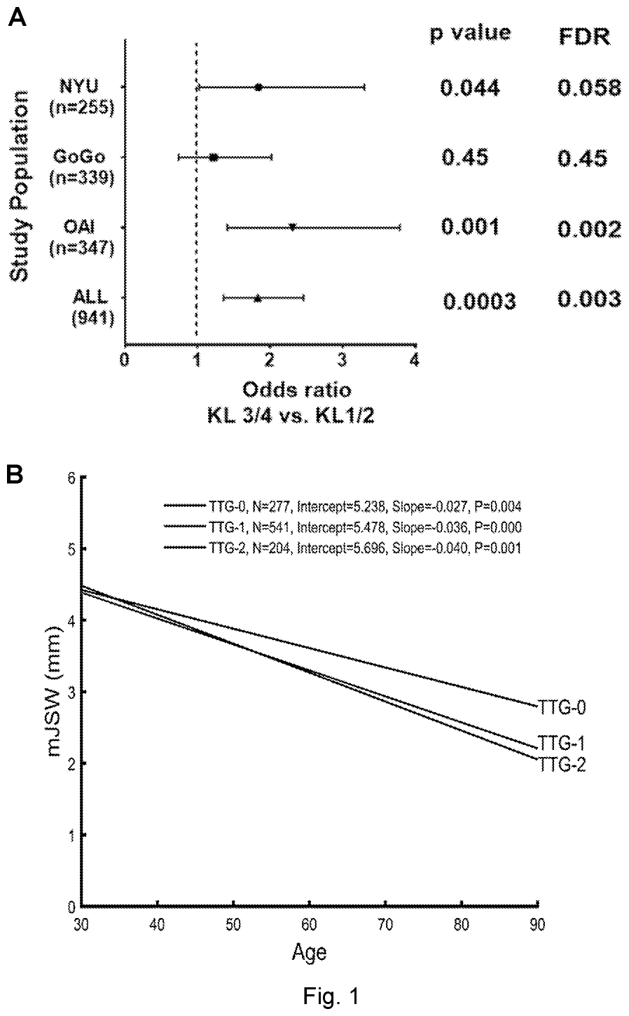

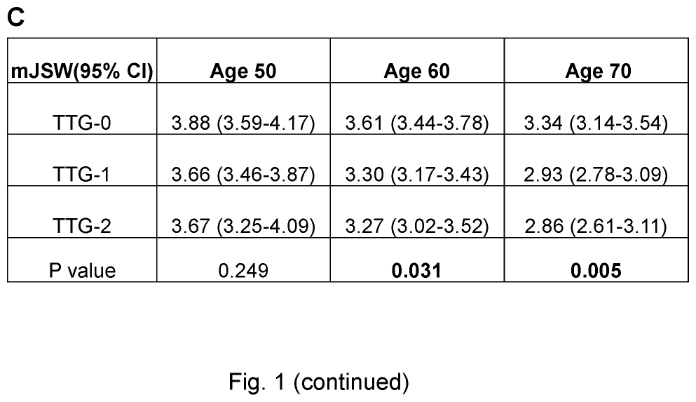

1. Frequency of IL1RN haplotypes across cohorts. The clinical, genetic, and demographic parameters in the three cohorts are shown in Table 1 (below).
The frequencies of the IL1RN TTG haplotypes, based on SNPs rs419598, rs315952, and rs9005, were similar across the three cohorts. For the combined cohort of 1066 participants, the frequencies of TTG-0, TTG-1, and TTG-2 were 26.3%, 42.2%, and 19.5%, respectively. The overall frequency of the CTA-1 or -2 haplotype was 12.0% but varied across cohorts (NYU 7%; GOGO 20%; OA 9%). Approximately 30% of the TTG-0 haplotype subjects in the combined cohort were CTA carriers. Unless otherwise specified in the data here reported, subjects designated TTG-0 to include carriers of the CTA allele.
2. The IL1RN TTG haplotype is associated radiographic severity. We first examined IL1RN TTG-0 versus TTG haplotypes (TTG-1 and TTG-2) for association with tibiofemoral radiographic OA severity as reported by KL scores and mean medial radiographic joint space width (mJSW). The percent of subjects with KL ¾ scores varied from 29% in GOGO to 48% in NYU to 57% in the OAI. As shown in a Forest plot (FIG. 1A), the IL1RN TTG haplotype was associated with an increased odds of more severe (KL ¾ vs. KL ½) radiographic knee OA compared to age-, sex- and BMI-matched knee OA patients with TTG-0 (Odds Ratio of 1.83; 95% CI 1.36-2.46; p=0.0003). In the GOGO cohort, although the TTG haplotype was associated with increased KL ¾, this finding did not reach statistical significance. Of note, there was a lower percentage of participants with KL ¾ OA severity (29.8%) in GOGO compared to the NYU (48.9%) and OA (57.7%) cohorts, which may have reduced the power to demonstrate statistical significance. We also note the higher frequency of the “protective” CTA haplotype in the GOGO population (GOGO 20%; NYU 7%; OA 9%), which could account for the lower percentage of subjects with severe rOA, as we have reported (8).

We next assessed radiographic severity by minimal medial joint space width (mJSW). Table 2 (below) shows that relative to TTG-0, carriage of TTG-1 or TTG-2 was associated with a TTG “dose-dependent” decrease in mJSW in each individual population. In addition, linear regression analysis demonstrated that compared to TTG-0, the TTG-1 or 2 haplotypes were significantly associated with decreased mJSW (Table 7, Panel A). This TTG dose effect was also observed for KL severity (Table 7, Panel B). The risk haplotype TTG carriers did not associate with either WOMAC or VAS pain in the NYU or OA cohort.

We next performed a more detailed analysis of the effects of different combinations of CTA and TTG haplotypes on mJSW. As shown in Table 3 (below), any combination of CTA-1 or CTA-2 was associated with wider mJSW compared to TTG-2 or TTG-1. For example, the mJSW for CTA-2 and CTA-1 or -2 carriers was 3.67 (1.3) and (3.51 (1.31), respectively. In comparison, the mean mJSW for TTG-2 and TTG-1 or 2 carriers was 3.07 (1.62) and 3.19 (1.57), respectively. The differences between CTA and TTG mJSW were significant even after adjustment for common covariates age, sex, and BMI for each of these conditions (Table 3).

3. IL1RN TTG haplotype predicts age-related rOA. We next evaluated the interaction between age, mJSW, and IL1RN genotypes relative to radiographic severity. As shown in FIG. 1B, linear regression analysis demonstrated that tibiofemoral mJSW decreased with age in all haplotype groups at all ages studied, p=0.0001, and p=0.002, respectively. As shown in FIG. 1C, the mean tibiofemoral mJSW was lower in TTG-2 (CTA-0) carriers at the specific ages analyzed: 50, 60, and 70 years. For example, at age 70, mean mJSW was 3.34 mm in TTG-0 vs. 2.86 mm in TTG-2 (p<0.005).

We further analyzed whether rOA was associated with IL1RN risk haplotype after adjustment for risk covariates (age, sex, and BMI) in the regression model. As shown in Table 8, IL1RN risk haplotype carriers (TTG-1 or 2) had significantly narrower tibiofemoral mJSW [beta coefficient 0.36 (±SD 0.10), p=0.001] compared to TTG-0 carriers; the association remained significant after adjustment for the covariates. In gender-specific analyses, we show that both male and female carriers of either TTG-1 or TTG-2 carriers had narrower mJSW compared to TTG-0 (Table 9). In addition, we also performed mJSW and IL1RN genotypes relative to radiographic severity in Blacks and Hispanics. Consistent with other populations, TTG-1 or TTG-2 carriers had narrower mJSW compared to TTG-0 (Tables 10 and 11) although the differences approached, but did not reach significance due to small sample size in each of the haplotype groups.

4. IL1RN TTG haplotype predicts the risk of incident rOA. We examined participants from the original Incidence Subcohort of the OAI, selecting the subgroup without clinical or radiographic evidence of knee OA at baseline. Based on the criteria detailed, 792 participants in the Incidence Subcohort qualified for this analysis (FIG. 2). From these, we identified 101 cases who developed either radiographic or symptomatic tibiofemoral radiographic knee OA within 2-4 years of baseline. Using a nested case-control approach, we selected 101 controls from the OA Incidence Subcohort who did not develop either pain or radiographic tibiofemoral OA (>KL1) over a similar period matched for age, sex and BMI. Subjects were followed for up to 8 years. Table 4 (below) shows that the presence of the IL1RN TTG-2 haplotype significantly increased the risk of incident knee rOA [OR=4.13 (1.75-9.72); p=0.001]. After adjustment for age, sex and BMI with a logistic regression model, carriage of the TTG haplotype remained positively and significantly associated with incident rOA (beta coefficient=1.38; 95% CI 0.48-2.28; p=0.002).
5. TTG risk haplotype is associated with decreased plasma IL-1Ra levels in patients with osteoarthritis. Genetic variants of IL1RN have been associated with plasma levels of IL-1ra and that IL1RN splice variants regulate intracellular IL-1Ra protein trafficking (21-24). We measured plasma IL-1Ra concentrations in the NYU cohort, where heparinized plasma samples were collected. Mean plasma IL1Ra protein concentrations in TTG-2 carriers were lower than in CTA-2 carriers (346.50 vs. 479.45, pg/ml, p=0.05, respectively). In this subset of patients, carriers of the CTA-2 haplotype had wider mean (SD) mJSW than did TTG-2 carriers [3.28 (1.46) vs. 2.60 (1.67 mm), p<0.046 age-, sex- and BMI-adjusted]. This was despite the fact that CTA-2 carriers were a mean 6 years olderthan the TTG-2 carriers (62.33, (10.96) vs. 68.94, (9.92) years, p<0.08), consistent with an age-evident “protective” effect of CTA on rOA (8).

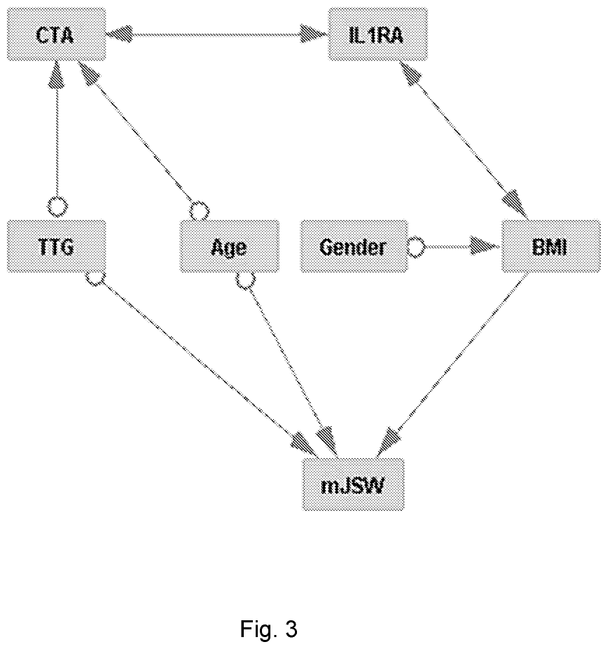

We also performed a causal analysis to determine relationships among IL1RN TTG, CTA haplotypes, age, sex, BMI, IL-1Ra, and mJSW. As shown in FIG. 3, the CTA haplotype and BMI, but not age, are independently associated with plasma IL-1Ra. The causal analysis also indicated that the TTG haplotype directly associated with mJSW. As expected, both age and BMI associated with mJSW, and these effects were independent of the TTG haplotype.

6. TTG risk haplotype is associated with decreased plasma IL-1Ra levels, increased DAS28 and inflammatory markers in patients with rheumatoid arthritis. We examined plasma samples from new-onset and DMARD-untreated patients with RA, followed at NYU (Jain et al., Id.; Sher et al., Id.). As shown in Table 5 (below), carriers of the TTG risk haplotype exhibited lower levels of plasma IL-1Ra and the sIL-6 receptor antagonist alpha (sIL-6Ra) than age-, BMI- and sex-matched individuals with RA. Conversely, in TTG carriers plasma IL-6, and hsCRP were higher. Clinically, carriers of the TTG haplotype exhibited greater disease activity (DAS28).
7. Chondrocytes of IL1RN TTG carriers exhibit decreased secreted and increased intracellular IL-1Ra expression. We next examined the relationship between TTG haplotypes and IL-1Ra production by chondrocytes. Chondrocytes were isolated from patients undergoing total joint replacement surgery at NYU, as described (19). Cell lysates and matched supernatants were analyzed for IL-1Ra protein concentrations after 24 h culture in the presence or absence of IL-1β. As shown in Table 2, following exposure to IL-β, basal levels of secreted IL-1Ra did not increase in TTG carriers, whereas intracellular concentrations of IL-1Ra in TTG chondrocytes were markedly increased. In contrast, chondrocytes obtained from TTG-0 individuals significantly increased the production of both intracellular and extracellular IL-1Ra following stimulation with IL-1β.

### E. TABLES

TABLE 1
Demographic and radiographic characteristic features of symptomatic
knee osteoarthritis patients from NYU, GOGO and OAI cohorts.

| | | | | | | | Haplotype % frequencies | | | | |
|---|---|---|---|---|---|---|---|---|---|---|---|
| | Age | Sex (% | | Ethnicity | KL ¾ | mJSW | | CTA-½ | TTG-1 | | CTA-1 |
| Cohort | in years | females) | BMI | (% Caucasians) | (%) | in mm | TTG-0 | (TTG-0) | (CTA-0) | TTG-2 | (TTG-1) |
| NYU | 61.4 | 62.7% | 26.41 | 64% | 48.9% | 3.21 | 22.3% | 8.0% | 46.0% | 15.3% | 10.5% |
| (n = 372) | (±10.4) | | (±3.52) | | | (±1.53) | | | | | |
| GOGO | 67.0 | 75.5% | 26.8 | 100% | 29.8% | 3.35 | 32.0% | 20.0% | 41.0% | 27.0% | 0 |
| (n = 339) | (±8.2) | | (±3.40) | | | (±1.4) | | | | | |
| OAI | 61.6 | 56.9% | 29.95 | 80% | 57.7% | 3.54 | 24.2% | 9.3% | 40.0% | 17.2% | 16.4% |
| (n = 355) | (±9.0) | | (±4.90) | | | (±1.65) | | | | | |
| Combined | 62.7 | 64.4% | 27.36 | 68.5% | 43.2% | 3.24 | 26.3% | 12.0% | 42.2% | 19.5% | 9.0% |
| (n = 1066) | (±9.9) | | (±4.15) | | | (±1.61) | | | | | |

Details are shown of the mean (±SD) age, body mass index (BMI) and medial joint space width (mJSW) in millimeters (mm), as well as percentage of females, Caucasians, radiographic Kellgren-Lawrence (KL) score 3 or 4 distribution, and IL1RN TTG haplotype frequency distribution. The TTG-0 groups also include CTA-½ haplotype group. Haplotypes TTG-0 or TTG-1 or TTG-2, respectively, represent carriers of 0 or 1 or 2 copies of IL1RN haplotype produced using 3 IL1RN SNRs (rs419598, rs315952 and rs9005). Cohort abbreviations: NYU = New York University School of Medicine; GOGO = Genetics of Generalized Osteoarthritis study, Duke University; OAI = Osteoarthritis Initiative.

TABLE 2
Association of IL1RN haplotype (TTG) with radiographic mean medial joint space width
(mJSW) in three combined (NYU, GOGO and OAI) cohorts of symptomatic knee OA patients.

| Cohorts | TTG-0 | TTG-1 | TTG-2 | Beta (95% CI) | p value | FDR |
|---|---|---|---|---|---|---|
| NYU | 3.43 (±1.44) | 3.28 (±1.45) | 2.60 (±1.70) | −0.39 | 0.0030 | 0.0046 |
| [n = 372] | [n = 83] | [n = 209] | [n = 57] | (−0.64 to −0.13) | | |
| GOGO | 3.67 (±1.31) | 3.08 (±1.51) | 3.35 (±1.38) | −0.18 | 0.0049 | 0.0950 |
| [n = 339] | [n = 111] | [n = 138] | [n = 90] | (−0.38 to 0.02) | | |
| OAI | 3.40 (±1.46) | 3.20 (±1.63) | 3.09 (±1.81) | −0.16 | 0.4787 | 0.2364 |
| [n = 355] | [n = 86] | [n = 200] | [n = 61] | (−0.42 to 0.10) | | |
| All | 3.52 (±1.40) | 3.20 (±1.53) | 3.07 (±1.63) | −0.23 | 0.0023 | 0.0021 |
| [n = 1066] | [n = 280] | [n = 547] | [n = 208] | (−0.37 to −0.10) | | |
| All meta-analysis | — | — | — | −0.23 | 0.0008 | 0.0021 |
| [n = 1066] | | | | (−0.37 to −0.10) | | |

Medial joint space width (mJSW) data are presented in millimeters as mean (±SD) unless otherwise indicated; number of subjects in each group is represented in square brackets below each value. Haplotypes TTG-0 or TTG-1 or TTG-2, respectively, represent carriers of 0 or 1 or 2 copies of IL1RN haplotype produced using 3 IL1RN SNPs (rs419598, rs315952 and rs9005). Linear regression model [beta and 95% confidence interval (CI)] was performed and p value was adjusted by false discovery rate (FDR). The last row indicates the meta-analysis of all three cohorts by including cohort as a covariate.

TABLE 3
IL1RN TTG-½ carriers consistently associated
with narrower joint space width in combined cohorts.

| | | | P | P | CTA-½ | | P |
|---|---|---|---|---|---|---|---|
| | CTA-2 | TTG-2 | value | value_ASB_Adj | (TTG-0) | TTG-2 | value |
| mJSW, mm | 3.67 | 3.07 | 0.0063 | 0.0072 | 3.51 | 3.07 | 0.0106 |
| | (±1.30) | (±1.62) | | | (±1.31) | (±1.62) | |
| Age, years | 65.01 | 65.03 | 0.9881 | — | 64.97 | 65.03 | 0.9498 |
| | (±8.24) | (±9.29) | | | (±8.95) | (±9.29) | |
| BMI | 27.15 | 27.74 | 0.2933 | — | 27.31 | 27.74 | 0.3272 |
| | (±4.02) | (±3.98) | | | (±3.66) | (±3.98) | |
| Sex | 36.76% | 35.1% | — | — | 33.6% | 35.1% | — |
| (% male) | | | | | | | |

| | P | CTA-½ | TTG-½ | P | P |
|---|---|---|---|---|---|
| | value_ASB_Adj | (TTG-0) | (CTA-0) | value | value_ASB_Adj |
| mJSW, mm | 0.0124 | 3.51 | 3.19 | 0.0304 | 0.0123 |
| | | (±1.31) | (±1.57) | | |
| Age, years | — | 64.97 | 63.64 | 0.1571 | — |
| | | (±8.95) | (±9.85) | | |
| BMI | — | 27.31 | 27.55 | 0.5478 | — |
| | | (±3.66) | (±4.20) | | |
| Sex | — | 33.6% | 33.4% | — | — |
| (% male) | | | | | |

Details are shown of the mean (±SD) age, body mass index (BMI) and medial joint space width (mJSW) in millimeters (mm), as well as percentage of males in each haplotype group in combined cohort (including NYU, GOGO and OAI). Haplotypes TTG-0 or TTG-1 or TTG-2, respectively, represent carriers of 0 or 1 or 2 copies of IL1RN haplotype produced using 3 IL1RN SNPs (rs419598, rs315952 and rs9005). Data are presented as mean (±SD) unless otherwise indicated. P values were determined by 2-sample t-test and adjusted for age, sex and BMI (ASB).

TABLE 4
IL1RN TTG haplotype increases risk of incident OA.

| | Age | | | | | OR (95% CI); | Beta ASB |
|---|---|---|---|---|---|---|---|
| | (years) | BMI | Sex | TTG-2 | TTG-0 | P value | adjusted |
| Cases | 62.6 ± 8.9 | 26.4 ± 3.3 | M = 31; | 48 | 16 | 4.13 | 1.38 |
| | | | F = 70 | (M = 14; | (M = 7; | (1.75-9.72); | (0.48-2.28); |
| | | | | F = 34) | F = 9) | 0.001 | 0.002 |
| Controls | 62.6 ± 8.8 | 26.3 ± 3.3 | M = 31; | 16 | 22 | | |
| | | | F = 70 | (M = 6; | (M = 0; | | |
| | | | | F = 10) | F = 22) | | |

Development of incident OA in cases was defined as development of frequent knee pain and radiographic OA (KL ≥1 or 2) in the same knee or in bilateral knees. Controls were individuals whose baseline KL = 0 or 1 did not change at follow up AND who did not develop frequent pain in either knee at 24, 36, 48, 72 and 96 months. Cases and controls were matched for age, sex and BMI. Estimates of odds ratio with 95% confidence intervals (CI) between severity of knee OA defined as KL ½ vs KL ¾ for haplotype rs419598, rs315952 and rs9005 “T-T-G” are shown. Haplotypes TTG-0 or TTG-1 or TTG-2, respectively, represent carriers of 0 or 1 or 2 copies of IL1RN haplotype produced using 3 IL1RN SNPs (rs419598, rs315952 and rs9005). Data for age and BMI are presented as mean ± SD; data for sex are presented as N of male and female. The odds ratios of patients falling into Case or Control groups vs. IL1RN haplotype were calculated using Fisher's exact test. Beta coefficient (and 95% CI) from logistic regressions were adjusted for age, sex and BMI (ASB).

TABLE 5
IL1RN TTG -risk haplotype predicts decreased IL-1Ra,
sIL-6R and increased IL-6, hsCRP accompanied by increased
disease activity (DAS28) in Rheumatoid arthritis.

| Biomarker | TTG-0 (n = 50) | TTG-½ (n = 95) | p value | FDR |
|---|---|---|---|---|
| Age | 44.81 ± 12.84 | 47.78 ± 13.64 | 0.19 | 0.19 |
| Sex | M = 9; F = 41 | M = 21; F = 74 | | |
| IL-1Ra (pg/ml) | 500.2 ± 275.1 | 386.8 ± 159.9 | 0.039 | 0.054 |
| IL-6 (pg/ml) | 8.23 ± 8.21 | 16.62 ± 19.9 | 0.012 | 0.028 |
| slL-6R alpha | 42.87 ± 11.02 | 36.70 ± 11.00 | 0.013 | 0.028 |
| hsCRP | 7.34 ± 6.6 | 14.41 ± 15.2 | 0.045 | 0.054 |
| DAS28 (2-10) | 4.9 ± 1.2 | 5.6 ± 1.4 | 0.014 | 0.028 |

Haplotypes TTG-½ or TTG-0 represent carriers of either IL1RN haplotype produced using 3 IL1RN SNPs (rs419598, rs315952 and rs9005). Details of IL-1Ra, IL-6, sIL-1Rα, hsCRP and DAS28 score and the mean age and sex in each haplotype group are shown. Data are presented as mean (±SD) unless otherwise indicated. Mann Whitney U test was used to analyze significance between groups, and differences with p<0.05 were considered significant.

TABLE 6
Linkage disequilibrium parameters D′ and r2 for IL1RN
single nucleotide polymorphisms (SNPs) rs419598, rs315952
and rs9005 for all three cohorts: New York University
School of Medicine (NYU), Genetics of Generalized Osteoarthritis
(GOGO) and Osteoarthritis Initiative (OAI).

| | D′/r² | | |
|---|---|---|---|
| SNP pair | NYU | GOGO | OAI |
| rs419598/rs315952 | 0.62/0.21 | 0.72/0.22 | 0.57/0.22 |
| rs419598/rs9005 | 0.81/0.61 | 0.86/0.75 | 0.72/0.67 |
| rs35952/rs9005 | 0.68/0.31 | 0.99/0.35 | 0.82/0.35 |

TABLE 7
IL1RN haplotype TTG association with mJSW and radiographic
severity in combined (NYU, GOGO, and OAI) cohorts.

| Panel A. IL1R/VSNP | mJSW: beta (±SE), |
|---|---|
| All Combined (n = 1066) | p value, FDR |
| TTG-0 vs. | −0.35 (0.11), |
| TTG-1 haplotype | p = 0.001, |
| | FDR = 0.004 |
| TTG-0 vs. | −0.45 (0.14), |
| TTG-2 haplotype | p = 0.001, |
| | FDR = 0.007 |

| Panel B: IL1RN SNP | KL severity: OR (95% CI), |
|---|---|
| All Combined (n = 1066) | p value, FDR |
| TTG-0 vs. | 1.53 (1.12-2.08), |
| TTG-1 haplotype | p = 0.008, |
| | FDR = 0.017 |
| TTG-0 vs. | 1.69 (1.27-2.24), |
| TTG-2 haplotype | p = 0.000, |
| | FDR = 0.001 |

In panel A, association of medial joint space width (mJSW) in millimeters (mm) with IL1RN haplotypes were studied by comparing the indicated haplotype (TTG-0) with TTG-1 or TTG-2 using linear regression analysis. In panel B, Patients were classified by radiographic severity of knee OA by stratifying the KL scores to compare KL 1 or 2 versus KL 3 or 4. Odds ratios (OR) of KL vs. IL1RN haplotype also shows the association of Kellgren-Lawrence (KL) score with IL1RN haplotype.

TABLE 8
Regression analysis of IL1RN haplotype (TTG) with
radiographic medial joint space width (mJSW) in
three cohorts of symptomatic knee OA patients.

ALL (n = 1022)
Beta (SE); P = value

mJSW (mm) (Baseline), TTG Haplotype (0 vs. ½)

| | |
|---|---|
| crude | −0.35 (0.11); 0.001 |
| adjusted for age, sex | −0.35 (0.10); 0.001 |
| adjusted for age, sex, BMI | −0.36 (0.10); 0.001 |

mJSW (mm) (Baseline), TTG Haplotype (0 vs 2)

| | |
|---|---|
| crude | −0.45 (0.14); 0.001 |
| adjusted for age, sex | −0.40 (0.14); 0.004 |
| adjusted for age, sex, BMI | −0.40 (0.14); 0.003 |

Medial joint space width (mJSW) in millimeters comparing the indicated haplotype (TTG-0) with TTG-1 or TTG-2. Haplotypes TTG-0 or TTG-1 or TTG-2, respectively, represent carriers of 0 or 1 or 2 copies of IL1RN haplotype produced using 3 IL1RN SNPs (rs419598, rs315952 and rs9005). Linear regression was performed unadjusted, adjusted for age and sex, as well as adjusted for age, sex and body mass index (BMI) together.

TABLE 9
Association of IL1RN haplotype (TTG) in male and
female with radiographic signal knee medial joint
space width (mJSW) in combined (NYU, GOGO, and
OAI) cohorts of symptomatic knee OA patients.

| mJSW (mm) (Baseline), TTG | Beta (SE), p value |
|---|---|
| Haplotype (0 vs ½) - Male | ALL (n = 355) |
| Crude | −0.48 (0.20), 0.018 |
| adjusted for age, sex | −0.54 (0.19), 0.006 |
| adjusted for age, sex, BMI | −0.50 (0.19), 0.009 |
| mJSW (mm) (Baseline), TTG | |
| Haplotype (0 vs 2) - Male | ALL (n = 164) |
| Crude | −0.58 (0.26), 0.027 |
| adjusted for age, sex | −0.51 (0.25), 0.042 |
| adjusted for age, sex, BMI | −0.49 (0.24), 0.047 |
| mJSW (mm) (Baseline), TTG | |
| Haplotype (0 vs ½) - Female | ALL (n = 667) |
| Crude | −0.28 (0.12), 0.021 |
| adjusted for age, sex | −0.27 (0.12), 0.025 |
| adjusted for age, sex, BMI | −0.28 (0.12), 0.023 |
| mJSW (mm) (Baseline), TTG | |
| Haplotype (0 vs 2) - Female | ALL (n = 317) |
| Crude | −0.38 (0.16), 0.018 |
| adjusted for age, sex | −0.34 (0.16), 0.033 |
| adjusted for age, sex, BMI | −0.34 (0.16), 0.034 |

Medial joint space width (mJSW) in millimeters comparing the indicated haplotype (TTG-0) with TTG-1 or TTG-2. Haplotypes TTG-0 or TTG-1 orTTG-2, respectively, represent carriers of 0 or 1 or 2 copies of IL1RN haplotype produced using 3 IL1RN SNPs (rs419598, rs315952 and rs9005). A linear regression model was performed and adjusted for age and sex, as well as for age, sex and body mass index (BMI) in the combined cohort.

TABLE 10
Association of IL1RN haplotype (TTG) in Blacks with radiographic
signal knee medial joint space width (mJSW) in combined
(NYU and OAI) cohorts of symptomatic knee OA patients.

| mJSW (mm) (Baseline), TTG | Beta (SE), p value |
|---|---|
| Haplotype (0 vs ½) | ALL (n = 82) |
| Crude | −0.48 (0.34), 0.165 |
| adjusted for age, sex | −0.46 (0.34), 0.183 |
| adjusted for age, sex, BMI | −0.44 (0.34), 0.201 |
| mJSW (mm) (Baseline), TTG | |
| Haplotype (0 vs 2) | ALL (n = 45) |
| Crude | −0.77 (0.44), 0.091 |
| adjusted for age, sex | −0.79 (0.44), 0.080 |
| adjusted for age, sex, BMI | −0.82 (0.45), 0.072 |

Medial joint space width (mJSW) in millimeters comparing the indicated haplotype (TTG-0) with TTG-1 or TTG-2. Haplotypes TTG-0 or TTG-1 or TTG-2, respectively, represent carriers of 0 or 1 or 2 copies of IL1RN haplotype produced using 3 IL1RN SNPs (rs419598, rs315952 and rs9005). A linear regression model was performed and adjusted for age and sex, as well as for age, sex and body mass index (BMI), in the combined cohort.

TABLE 11
Association of IL1RN haplotype (TTG) in Hispanic and
non-Hispanic with baseline radiographic signal knee
medial joint space width (mJSW) in combined (NYU
and OAI) cohorts of symptomatic knee OA patients.

| Hispanic | |
|---|---|
| mJSW (mm), TTG | Beta (SE); P value |
| Haplotype (0 vs ½) | ALL (n = 347) |
| crude | −0.39 (0.24); 0.106 |
| adjusted for age, sex | −0.33 (0.24); 0.160 |
| adjusted for age, sex, BMI | −0.38 (0.24); 0.108 |
| mJSW (mm), TTG | |
| Haplotype (0 vs 2) | ALL (n = 122) |
| crude | −0.56 (0.29); 0.052 |
| adjusted for age, sex | −0.37 (0.27); 0.176 |
| adjusted for age, sex, BMI | −0.42 (0.27); 0.126 |

| Non-Hispanic | |
|---|---|
| mJSW (mm), TTG | Beta (SE); P value |
| Haplotype (0 vs ½) | ALL (n = 587) |
| crude | −0.33 (0.17); 0.057 |
| adjusted for age, sex | −0.29 (0.17); 0.088 |
| adjusted for age, sex, BMI | −0.31 (0.17); 0.066 |
| mJSW (mm), TTG | |
| Haplotype (0 vs 2) | ALL (n = 211) |
| crude | −0.63 (0.22); 0.005 |
| adjusted for age, sex | −0.49 (0.21); 0.022 |
| adjusted for age, sex, BMI | −0.49 (0.21); 0.021 |

Medial joint space width (mJSW) in millimeters comparing the indicated haplotype (TTG-0) with TTG-1 or TTG-2. Haplotypes TTG-0 or TTG-1 or TTG-2, respectively, represent carriers of 0 or 1 or 2 copies of IL1RN haplotype produced using 3 IL1RN SNPs (rs419598, rs315952 and rs9005). A linear regression model was performed and adjusted for age and sex, as well as for age, sex and body mass index (BMI), in the combined cohort.

TABLE 12
The IL1RN risk haplotype TTG (1 or 2) associated with
decreased soluble IL-1Ra levels in chondrocytes

| Unstimulated, mean (±SD) | | | IL-1 induced, mean (±SD) | | |
|---|---|---|---|---|---|
| Extra- | Intra- | Ratio | Extra- | Intra- | Ratio |
| cellular | cellular | (s vs. | cellular | cellular | (s vs. |
| (s) | (ic) | ic) | (s) | (ic) | ic) |

IL1RN haplotype

| | | | | | | |
|---|---|---|---|---|---|---|
| TTG-1,2 | 5.05 | 7.93 | 1:1.2 | 6.19 | 33.71 | 1:5.4 |
| (n = 15) | (±8.1) | (±14.9) | | (±7.1) | (±22.0) | |
| TTG-0 | 1.46 | 1.41 | 1:1 | 10.36 | 12.28 | 1:1.2 |
| (n = 14) | (±2.2) | (±2.5) | | (±11.9) | (±8.4) | |

P value

| | | | | | | |
|---|---|---|---|---|---|---|
| TTG-1, 2 vs. | 0.05 | 0.06 | — | 0.22 | 0.07 | — |
| TTG-0 | | | | | | |
| TTG-1,2- | — | — | — | 0.62 | 0.04 | — |
| Con vs. IL-1 | | | | | | |
| TTG-0 - | — | — | — | 0.01 | 0.0005 | — |
| Con vs. IL-1 | | | | | | |

We analyzed total cell lysate for extracellular and cell-associated IL-1Ra protein expression in primary chondrocytes by ELISA. Haplotypes TTG-0 or TTG-1 or TTG-2, respectively, represent carriers of 0 or 1 or 2 copies of IL1RN haplotype produced using 3 IL1RN SNPs (rs419598, rs315952 and rs9005). Chondrocytes with IL1RN TTG-½ haplotype produced significantly lower levels of soluble IL1Ra than TTG-0 carriers in both spontaneous conditions and following 24h IL-1β induction. The data represent mean (±SD), for n = 15 individuals with TTG-½ and n = 14 individuals with TTG-0. Mann Whitney U test was used to analyze significance between groups, and differences with p < 0.05 were considered significant.

### F. DISCUSSION

The IL-1 gene cluster region has been associated with susceptibility to OA in various joints, but the results have been inconsistent (25-30). In this study of over 1000 individuals with symptomatic knee OA, we confirm that carriers of the IL1RN CTA haplotype (rs419598, rs315952 and rs9005) exhibit decreased age-dependent radiographic severity. Conversely, the TTG haplotype, lacking CTA, is associated with more severe rOA. Moreover, we now show that the IL1RN TTG haplotype confers significantly increased the risk for incident tibiofemoral knee OA. These data are consistent with recent literature suggesting a genetic association of IL1RN gene variations with rOA (16, 31).

Previous genetic studies of OA susceptibility or severity have not identified any compelling association with a particular locus, but rather support the polygenic contributions of multiple variants with small effect sizes. Therefore, the IL1RN associations that we describe are unique: no other gene of such prevalence (21.5% for TTG-2, 63%, for TTG-1 or -2) has been shown to be associated with such high-impact risk for age-dependent radiographic severity or risk of incident disease. These observations have clinical implications. Drug development in OA would benefit from genetic biomarkers that identify individuals at greater risk for more severe or incident OA (32). Stratification by IL1RN risk haplotype in clinical trial design and future personalized medicine strategies could identify subsets of anti-IL1 responders/non-responders based on IL1RN risk haplotypes, as has been described in juvenile systemic arthritis (Arthur et al., IL1RN Variation Influences Both Disease Susceptibility and Response to Recombinant Human Interleukin-1 Receptor Antagonist Therapy in Systemic Juvenile Idiopathic Arthritis. Arthritis Rheumatol, 2018. 70(8): p. 1319-1330).

Biological explanation for the “yin/yang” genetic effects of CTA vs. TTG on rOA. We have previously shown that individuals carrying the IL1RN CTA (TTG-0) haplotype had significantly lower synovial fluid levels of IL-10 and showed a trend towards lower levels of IL-1β and IL-6 (12). We here report that in patients with both OA and RA carriers of the TTG haplotype exhibit reduced plasma levels of IL-1Ra compared to CTA carriers. We provide evidence in chondrocytes that this may result from intracellular sequestration of IL-1Ra protein. These data are supported by prior studies that have shown that blood and cellular production of IL-1Ra are associated with various polymorphic loci within the IL1RN gene (22, 33). We postulate that the greater severity of rOA in carriers of the TTG haplotype results from impaired antagonism of chronic inflammatory IL-1α-driven processes that perpetuate cartilage destruction (1, 2).

We are also able to show that the association of the TTG haplotype with more severe disease is not limited to OA, but can be demonstrated in patients with rheumatoid arthritis. It is important to note that in RA, decreased plasma IL-1Ra, an endogenous “anti-inflammatory”protein, is accompanied by increased plasma IL-6 and hsCRP in association with increased clinical disease activity (DAS28).

There are several limitations to our study, since we restricted our analyses to participants with BMI<33 kg/m² with symptomatic knee osteoarthritis. The exclusion of participants with higher BMI was done to limit the confounding influences of both excessive mechanical contribution to OA severity as well as the known state of low grade chronic inflammation that often accompanies obesity and the metabolic syndrome (34). The restriction of our studies to knee OA resulted from the fact that standardized radiographs of other joints were not available in each of the study cohorts. The focus on knee OA also reduces the heterogeneity of the study population, which has been a recognized weakness of genetic studies in OA. Finally, we note that participants enrolled in our three cohorts were predominantly North American Caucasian. However, although the numbers were small, subset analysis of Black and Hispanic subjects did indicate a trend towards increased rOA severity in each that approached significance (Table 10 and Table 11).

In summary, we demonstrate that the IL1RN TTG haplotype identifies a substantial subset of individuals with knee OA who are at increased risk for age-dependent joint destruction and increased risk for incident OA. The observations, in both OA and RA, suggest that carriers of the IL1RN TTG haplotype experience more severe disease due to genetically determined impaired “anti-inflammatory” mechanisms.

### G. REFERENCES

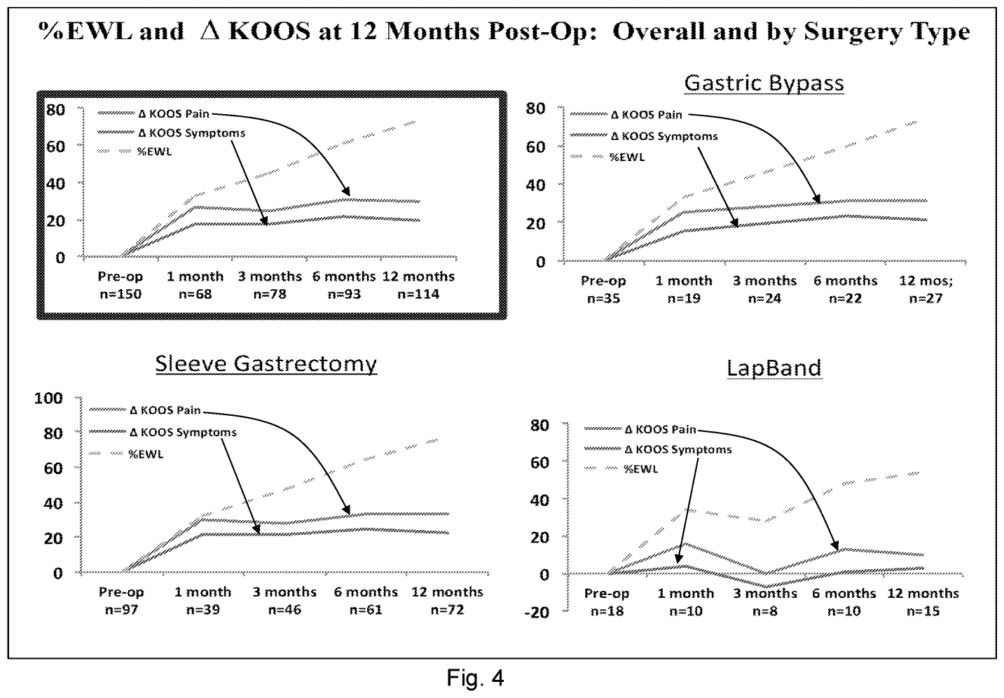

1. Attur M, Krasnokutsky S, Statnikov A, et al. Low-grade inflammation in symptomatic knee osteoarthritis: prognostic value of inflammatory plasma lipids and peripheral blood leukocyte biomarkers. Arthritis Rheumatol. 2015; 67(11):2905-2915.
2. Robinson W H, Lepus C M, Wang Q, et al. Low-grade inflammation as a key mediator of the pathogenesis of osteoarthritis. Nat Rev Rheumatol. 2016; 12(10):580-592.
3. Watson Levings R S, Broome T A, Smith A D, et al. Gene Therapy for Osteoarthritis: Pharmacokinetics of Intra-Articular Self-Complementary Adeno-Associated Virus Interleukin-1 Receptor Antagonist Delivery in an Equine Model. Hum Gene Ther Clin Dev. 2018; 29(2):90-100.
4. Attur M G, Dave M, Cipolletta C, et al. Reversal of autocrine and paracrine effects of interleukin 1 (IL-1) in human arthritis by type IL-1 decoy receptor. Potential for pharmacological intervention. J Biol Chem. 2000; 275(51):40307-40315.
5. Gonzalez A, Valdes A M. Big data boost for osteoarthritis genetics. Nat Rev Rheumatol. 2018; 14(7):387-388.
6. Zengini E, Hatzikotoulas K, Tachmazidou I, et al. Genome-wide analyses using UK Biobank data provide insights into the genetic architecture of osteoarthritis. Nat Genet. 2018; 50(4):549-558.
7. Rodriguez-Fontenla C, Calaza M, Evangelou E, et al. Assessment of osteoarthritis candidate genes in a meta-analysis of nine genome-wide association studies. Arthritis Rheumatol. 2014; 66(4):940-949.
8. Yau M S, Yerges-Armstrong L M, Liu Y, et al. Genome-Wide Association Study of Radiographic Knee Osteoarthritis in North American Caucasians. Arthritis Rheumatol. 2017; 69(2):343-351.
9. Fernandez-Moreno M, Rego I, Carreira-Garcia V, Blanco F J. Genetics in osteoarthritis. Curr Genomics. 2008; 9(8):542-547.
10. Loughlin J. Genetic contribution to osteoarthritis development: current state of evidence. Curr Opin Rheumatol. 2015; 27(3):284-288.
11. Reynard L N, Loughlin J. The genetics and functional analysis of primary osteoarthritis susceptibility. Expert Rev Mol Med. 2013; 15:e2.
12. Attur M, Wang H Y, Kraus V B, et al. Radiographic severity of knee osteoarthritis is conditional on interleukin 1 receptor antagonist gene variations. Ann Rheum Dis. 2010; 69(5):856-861.
13. Kraus V B, Jordan J M, Doherty M, et al. The Genetics of Generalized Osteoarthritis (GOGO) study: study design and evaluation of osteoarthritis phenotypes. Osteoarthritis Cartilage. 2007; 15(2):120-127.
14. Mercier C, Piperno M, Vignon E, Brandt K, Hochberg M, Hellio Le Graverand M P. In normal knees, joint space width (JSW) is correlated with the intermargin distance (IMD), a measure of medial tibial plateau alignment. Variations in IMD explain variability in JSW in serial radiographs. Joint Bone Spine. 2013; 80(2):183-187.
15. Attur M, Statnikov A, Samuels J, et al. Plasma levels of interleukin-1 receptor antagonist (IL1Ra) predict radiographic progression of symptomatic knee osteoarthritis. Osteoarthritis Cartilage. 2015; 23(11):1915-1924.
16. Kerkhof H J, Doherty M, Arden N K, et al. Large-scale meta-analysis of interleukin-1 beta and interleukin-1 receptor antagonist polymorphisms on risk of radiographic hip and knee osteoarthritis and severity of knee osteoarthritis. Osteoarthritis Cartilage. 2011; 19(3):265-271.
17. Castano-Betancourt M C, Evans D S, Ramos Y F, et al. Novel Genetic Variants for Cartilage Thickness and Hip Osteoarthritis. PLoS Genet. 2016; 12(10):e1006260.
18. Yau M S. Elucidating the Phenotypic and Genetic Architecture of Osteoarthritis Progression [DISSERTATION]: Human Genetics, University of Maryland, Baltimore; 2015.
19. Attur M, Yang Q, Shimada K, et al. Elevated expression of periostin in human osteoarthritic cartilage and its potential role in matrix degradation via matrix metalloproteinase-13. FASEB J. 2015; 29(10):4107-4121.
20. Katsara O, Attur M, Ruoff R, Abramson S B, Kolupaeva V. Increased Activity of the Chondrocyte Translational Apparatus Accompanies Osteoarthritic Changes in Human and Rodent Knee Cartilage. Arthritis Rheumatol. 2017; 69(3):586-597.
21. Rafiq S, Stevens K, Hurst A J, et al. Common genetic variation in the gene encoding interleukin-1-receptor antagonist (IL-1RA) is associated with altered circulating IL-1RA levels. Genes Immun. 2007; 8(4):344-351.
22. Matteini A M, Li J, Lange E M, et al. Novel gene variants predict serum levels of the cytokines IL-18 and IL-1ra in older adults. Cytokine. 2014; 65(1):10-16.
23. Ramirez-Perez S, De la Cruz-Mosso U, Hernandez-Bello J, et al. High expression of interleukine-1 receptor antagonist in rheumatoid arthritis: Association with IL1RN*2/2 genotype. Autoimmunity. 2017; 50(8):468-475.
24. Redlitz K H, Yamshchikov V F, Cominelli F. Differential contribution of IL-1Ra isoforms to allele-specific IL-1Ra mRNA accumulation. J Interferon Cytokine Res. 2004; 24(4):253-260.
25. Loughlin J, Dowling B, Mustafa Z, Chapman K. Association of the interleukin-1 gene cluster on chromosome 2q13 with knee osteoarthritis. Arthritis Rheum. 2002; 46(6):1519-1527.
26. Moos V, Rudwaleit M, Herzog V, Hohlig K, Sieper J, Muller B. Association of genotypes affecting the expression of interleukin-1beta or interleukin-1 receptor antagonist with osteoarthritis. Arthritis Rheum. 2000; 43(11):2417-2422.
27. Smith A J, Keen L J, Billingham M J, et al. Extended haplotypes and linkage disequilibrium in the IL1R1-IL1A-IL1B-IL1RN gene cluster: association with knee osteoarthritis. Genes Immun. 2004; 5(6):451-460.
28. Kanoh T, Hasegawa Y, Masui T, Yamaguchi J, Ishiguro N, Hamajima N. Interleukin-1beta gene polymorphism associated with radiographic signs of osteoarthritis of the knee. J Orthop Sci. 2008; 13(2):97-100.
29. Moxley G, Han J, Stern A G, Riley B P. Potential influence of IL1B haplotype and IL1A-IL1B-IL1RN extended haplotype on hand osteoarthritis risk. Osteoarthritis Cartilage. 2007; 15(10):1106-1112.
30. Moxley G, Meulenbelt I, Chapman K, et al. Interleukin-1 region meta-analysis with osteoarthritis phenotypes. Osteoarthritis Cartilage. 2010; 18(2):200-207.
31. Wu X, Kondragunta V, Kornman K S, et al. IL-1 receptor antagonist gene as a predictive biomarker of progression of knee osteoarthritis in a population cohort. Osteoarthritis Cartilage. 2013; 21(7):930-938.
32. Karsdal M A, Christiansen C, Ladel C, Henriksen K, Kraus V B, Bay-Jensen A C. Osteoarthritis—a case for personalized health care? Osteoarthritis Cartilage. 2014; 22(1):7-16.
33. Gordon A, Kiss-Toth E, Stockley I, Eastell R, Wilkinson J M. Polymorphisms in the interleukin-1 receptor antagonist and interleukin-6 genes affect risk of osteolysis in patients with total hip arthroplasty. Arthritis Rheum. 2008; 58(10):3157-3165.
34. Pottie P, Presle N, Terlain B, Netter P, Mainard D, Berenbaum F. Obesity and osteoarthritis: more complex than predicted! Ann Rheum Dis. 2006; 65(11):1403-1405.
II. IL1RN Polymorphism Predicts Weight Loss, Inflammatory Biomarker Changes and Knee Osteoarthritis Pain Relief after Bariatric Surgery
   A. Purpose: Symptomatic knee osteoarthritis (SKOA) patients with obesity who undergo bariatric surgery experience knee pain relief, though the reduced mechanical load explains only part of the improvement. A reduction in inflammatory biomarkers from adipose tissue may also impact pain. We previously identified an IL1RN haplotype (TTG; rs419598, rs315952, and rs9005) that associates with OA severity and inflammatory markers. We aimed to determine whether TTG distinguishes patients who lose more weight and have more significant decreases in inflammation with greater knee OA pain relief.
   B. Methods: From 2013-2019 we enrolled patients ≥30 years old with BMI≥30 kg/m2 and painful knee OA who planned surgical (sleeve gastrectomy, gastric bypass, or laparoscopic band) or medical weight loss (MWL) at Bellevue Hospital or NYU Langone Health. Patients with lupus, rheumatoid arthritis, or psoriatic arthritis were excluded. Weight-bearing knee x-rays assessed OA severity to confirm a Kellgren-Lawrence grade of at least 1 (scale 0-4). Participants completed the Knee Injury and Osteoarthritis Outcomes (KOOS) questionnaire and provided blood at baseline and 1, 3, 6, and 12 months. Patients were genotyped to determine whether they carried 1 or 2 copies of the TTG haplotype (TTG-½) or none (TTG-0). Sleeve was the most common weight loss intervention, therefore our analysis is focused on this surgical subset to minimize variable effects on weight and biomarkers.
   C. Results: We enrolled 113 patients (95 F, 18 M) with painful knee OA prior to their weight loss intervention. The mean age, BMI, and KOOS pain at baseline were 50.3±12.0 years, 44.8 8.9 kg/m², and 48.4±18.2 (0-100, with 100=no pain). Of 113 patients, 48 underwent sleeve, 20 bypass, 9 laparoscopic banding, 12 did not have the surgery, and 24 pursued medical weight loss. The 77 who completed surgery had a mean % excess weight loss (% EWL) of 51.7 after 6 months, with significant decreases in hsCRP (4.4 mg/L) and leptin (32.8 ng/dL), and mean KOOS pain improvement of 22.4 (MCID=16.7). The corresponding changes for patients who tried various MWL regimens were modest at best. We obtained the IL1RN haplotype for 45 of the 48 sleeve patients, and found 34 (70.8%) carried the TTG-1 or TTG-2 haplotype while 11 were TTG-0 (with similar baseline age, BMI, and KOOS for the two groups). At each follow-up time point through 6 months (FIG. 4), TTG-½ patients had more difficulty losing weight than the TTG-0 group (p<0.005 by ANOVA), with corresponding smaller reductions in hsCRP (p=0.36) and leptin (p=0.006). TTG-½ carriers also reported less KOOS pain relief relative to the TTG-0 group (p=0.021), markedly at 1 and 3 months with some improvement later. All of these findings held true when plotting data from the 23 patients (18 TTG-½, 5 TTG-0) who completed each of the followup visits.
   D. IL1RN haplotypes predict % EWL and markers of obesity-associated inflammation following sleeve gastrectomy. Since SG is the most common bariatric surgical procedure both at NYU and nationwide, and was the bariatric surgery most represented in our NYU symptomatic knee OA Bariatric cohort, we have focused on this surgery. We genotyped and examined 53 patients who had undergone SG. Table 13 shows baseline data according to the IL1RN haplotype (TTG-0 copies vs. TTG-1 or 2 copies).

TABLE 13
IL1RN TTG-½ risk haplotype predicts decreased % EWL, increased
hsCRP, leptin and decreased IL-1Ra in 53 patients with obesity undergoing SG.

| | baseline | 1 Month | 3 Months | 6 Months | 12 Months | ANOVA |
|---|---|---|---|---|---|---|
| | % Excessive weight loss (% EWL) | | | | | |
| TTG-0 | | 31.63 | 50.01 | 72.54 | 80.74 | 0.014 |
| (n = 14) | | (13.1) | (17.4) | (21.6) | (27.4) | |
| TTG-½ | | 23.79 | 43.72 | 55.39 | 67.61 | |
| (n = 39) | | (11.2) | (15.3) | (19.1) | (26.1) | |
| | hsCRP (mg/dl) | | | | | |
| TTG-0 | 5.02 | 2.36 | 2.70 | 2.95 | 1.0 | 0.25 |
| | (2.8) | (0.6) | (2.1) | (3.7) | (0.1) | |
| TTG-½ | 7.52 | 6.52 | 5.21 | 4.88 | 2.09 | |
| | (4.0) | (3.3) | (3.9) | (4.0) | (2.2) | |
| | Leptin (pg/ml) | | | | | |
| TTG-0 | 75.98 | 34.97 | 20.02 | 17.71 | 31.56 | 0.002 |
| | (23.1) | (21.7) | (14.8) | (8.7) | (12.1) | |
| TTG-½ | 93.80 | 49.30 | 39.81 | 36.74 | 27.99 | |
| | (32.5) | (28.8) | (21.9) | (20.9) | (23.3) | |
| | IL-1Ra pg/ml | | | | | |
| TTG-0 | 1324.00 | 1490.54 | 968.10 | 648.80 | 502.83 | 0.018 |
| | (1222.2) | (785.6) | (703.0) | (395.2) | (369.8) | |
| TTG-½ | 795.29 | 1335.20 | 853.52 | 553.21 | 316.24 | |
| | (875.1) | (875.1) | (729.8) | (277.4) | (154.6) | |

Haplotypes TTG -½ or TTG-0 represent carriers of either IL1RN haplotype produced using 3 IL1RN SNPs (rs419598, rs315952, and rs9005). Data are presented as mean (±SD) of % EWL, hsCRP, Leptin and IL-1Ra for each haplotype group. ANOVA was performed to find trend for significant effect by surgery on biomarkers between two haplotype group. Age, BMI and gender were similar between haplotypes.

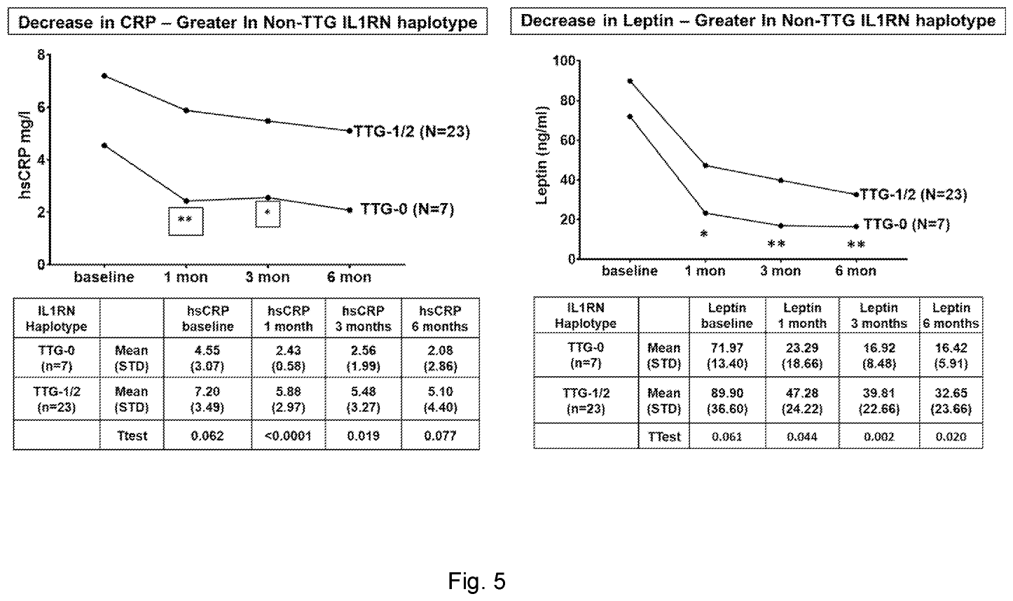

As shown, hsCRP and leptin were detected at higher and IL-1Ra lower levels in TTG-½ vs. TTG-0 carriers at baseline. This table also compares % EWL and decreases in the three inflammatory biomarkers between the IL1RN haplotypes over 12 months of follow up. At each follow-up time point, the % EWL was lower in TTG-½ carriers than TTG-0 (p<0.01 by ANOVA). Table 3 also shows that the decreases of hsCRP, leptin and IL-1Ra were smaller in the TTG carriers following SG. Of the 53 patients reported in Table 3, a full data set at each time point through 6 months was available for 30 patients. These data are shown in FIG. 2, where significant differences between haplotypes were observed for both hsCRP and leptin at each time point. While the absolute drops in these biomarkers from 0 to 6 months are similar for TTG-½ and TTG-0, the percent improvement from 0 to 6 months is significantly lower for TTG-½ carriers (p<0.05). Thus, these preliminary data clearly indicate that TTG-½ carriers respond less well to SG in association with less effective improvement in parameters of inflammation. The data are consistent with an interpretation that lower IL-1Ra levels in TTG carriers impair endogenous anti-inflammatory mechanisms, resulting in increased obesity-associated inflammation. See also FIG. 5. In FIG. 5, IL1RN genetic polymorphism associated with significant decrease (baseline—6 months) in serum highly sensitive c-reactive protein (hsCRP) and leptin following sleeve surgery in patients with obesity. IL1RN non-TTG carriers presented with lower hsCRP and leptin at baseline relative to TTG carriers and significantly decreased (y-axis) following surgery over a period of time compared to TTG carriers who presented with higher hsCRP and leptin. Higher inflammation in IL1RN TTG carriers associated with relatively less weight loss following bariatric surgery.
D. Conclusions: SKOA patients with obesity achieve marked excess weight loss, reductions in inflammatory mediators, and knee pain relief with bariatric surgery. The subset of patients with the TTG-0 IL1RN haplotype demonstrated more significant and/or rapid improvement in each of these outcomes, suggesting a potential predictor of which OA patients will have a more successful response to bariatric surgery.

### III. Genetic Polymorphisms of IL1RN Predict Post-Bariatric Changes in % EWL and Insulin Resistance (HbA1c and HOMA-IR).

A. Introduction The IL1RN TTG haplotype intensifies obesity-associated inflammation, which impedes weight loss and improvement of insulin resistance. IL1RN haplotypes (TTG-2 or TTG-1 copies vs. TTG-0 copies) are determined from DNA obtained from patients followed prospectively by investigators of the NYU Langone Comprehensive Program on Obesity. Following SG, % EWL is assessed at 3, 12 and 24 months after surgery as well as yearly 3-5 years post-surgery. Measures of insulin resistance are performed at the same time points.

### B. Patient Populations and Methods

1. Inclusion/Exclusion Criteria: Subjects between the ages of 18-75 years old who are candidates for bariatric surgery and scheduled to undergo the SG procedure, able to communicate, read, and write in English or Spanish, and able to give voluntary, written informed consent are included. Participants with: prior bariatric surgery, insulin dependent diabetes mellitus (on insulin), inability to discontinue the use of aspirin or non-steroidal anti-inflammatory medications (NSAIDs) for 72 hours before study testing, chronic gastrointestinal disorder (e.g. inflammatory bowel disease, ulcerative colitis, Crohn's or celiac disease), and human immunodeficiency virus (HIV) and/or medications to treat HIV are excluded. Subjects are recruited from Bellevue and Tisch hospitals. Each of these sites performs over 1000 bariatric surgical operations each year and more than ⅔ of the procedures performed at these sites are SGs. This cohort represents a diverse patient population.
2. Genomic DNA isolation and IL1RN genotyping: Genomic DNA is isolated and genotyped for IL1RN SNPs (rs419598, rs315952 and rs9005) as described above.
3. Baseline assessments. Demographic, clinical, and laboratory measurements are performed in all patients. Clinical specimens (blood, plasma and serum) for biomarker analysis are obtained and analyzed as described below. Specific markers of interest include IL-1Ra, IL-1, IL-6, sRAGE, hsCRP, MCP-1 (CCL2), leptin, adiponectin and ghrelin. Serum samples are assayed using commercial kits. To obtain unbiased determination of hormones and other cytokines baseline and all follow-up samples are analyzed in duplicate or triplicate as a group, using ELISA assay system to reduce variability, reduce batch variations and increase rigor and reproducibility. Measures of precision of each assay are performed using the lower limit of detection (LLOD), which represents the calculated concentration of the signal that is 2.5 standard deviations over the zero calibrators. Intra-assay (within-run) variation is determined by calculating % CV for all samples run on each individual plate and then taking the mean of those % CVs. Inter-assay (between-run) variation is determined by calculating the % CV of all control values run during the study (SD/Mean*100). For the purpose of conservative statistical analyses, any value of zero or below the level of detection is replaced with ½ LLOD.
4. Intraoperative samples: Intraoperative visceral and subcutaneous fat is collected on a subset of 40 patients. 1 month post surgery: Subcutaneous fat biopsies are collected on a subset of 30 patients.
5. Blood HbA1c and Homeostatic Model Assessment of Insulin Resistance (HOMA-IR) assay: Fasting glucose and fasting insulin levels are measured and then HOMA-IR is calculated as a measure of Insulin sensitivity.

### Findings

### C. Results

The IL1RN TTG haplotype formed from three SNPs (rs419598, rs315952, and rs9005) is found to be a predictive genetic marker of reduced response to SG surgery. Increases of % EWL following bariatric surgery are observed to be approximately 20% EWL at 3 months and continue to improve and stabilize by one year. Patients who carry the IL1RN TTG haplotypes are observed to exhibit a 15-20% less improvement of EWL than non-carriers. The improvement in inflammatory biomarkers after SG is observed to be significantly less in TTG carriers. As a consequence, TTG carriers are observed to have significantly less improvement of insulin resistance. A causal analysis of the multiple biomarkers studied indicates the interaction among pathways, as well as provides insights regarding the prominence of one or more biomarkers primarily responsible for differential effects of the IL1RN haplotypes on % EWL or insulin resistance.

IV. Characterization of Adipose Tissue Pre- and Post-Bariatric Surgery with Respect to Inflammatory Infiltrate and Gene Expression in Patients with or without the IL1RN TTG Risk Haplotype (Rs419598, Rs315952 and Rs9005)

### A. Introduction

Adipose tissue microenvironment of patients with obesity is composed of macrophages, precursor and hypertrophic adipocytes, and other immune cells that predominantly produce pro-inflammatory cytokines, such as IL-6, IL-1β, and TNF-α, for chronic, low-grade inflammation. Immunohistochemistry and single cell (sc) RNAseq studies of fresh visceral and subcutaneous adipose tissue obtained at SG (and subcutaneous tissue 1 month thereafter) are performed to assess cellular components. Cell-specific variations of IL1RN haplotype regulation of gene expression in the stromovascular fraction of adipose tissue, focusing on CD64+ proinflammatory and CD206+ anti-inflammatory adipose tissue macrophages are elucidated. The cellular source of IL1RN whose gene product, IL-1Ra, is so strikingly elevated in obesity, is determined. An assessment is made as to whether IL1RN haplotypes differentially affect changes in white adipose tissue (WAT) gene expression.

### B. Research Design and Methods

1. Intraoperative and Subcutaneous Adipose Tissue Aspiration Biopsy: Thirty newly recruited patients (equally divided between the IL1RN TTG and TTG-0 haplotypes) with obesity referred for SG are studied. Intraoperative visceral and subcutaneous WAT biopsies are collected and processed. Five grams of visceral WAT are collected at the time of surgery and processed immediately for the endpoints below. Specifically 2 g of subcutaneous WAT accessible near the laparoscopic port sites are collected for further analyses. Subcutaneous WAT aspiration biopsies at 6 weeks postop are performed on the contralateral (left) side to minimize intra-individual variation. Subcutaneous WAT is collected using a liposuction trocar (3-5 g total), rinsed in cold saline, filtered using a coarse 1 mm filter and allocated for analyses including immunohistochemistry and gene expression. Adipose tissue immunohistochemistry with H&E and CD68 staining is used to quantify CLS/cm² index, macrophage content and adipocyte diameters.

In collaboration with our NYU Experimental Pathology Core, a computer vision algorithm that facilitates the quantification of macrophage infiltration and adipocyte diameter, confirming the expected increase in macrophage content and decreased adipocyte diameter following bariatric weight loss in subjects with obesity and knee OA is employed. Further, stromovascular fraction is isolated by collagenase digestion of 2 g of obtained subcutaneous WAT using the CD45 marker for immune cells, with focus on CD14 (macrophage), CD64 (pro-inflammatory macrophages), and CD206 (anti-inflammatory macrophages).

2. Single Cell RNA Sequencing: To obtain a global and detailed analysis of circulating WBCs, CD45+ cells are isolated using the BD FACS Aria IIu SORP; scRNAseq is used to assess gene expression in cells that are CD14+(monocytes), pro-inflammatory CD64+, or anti-inflammatory CD206+ subpopulations postoperatively in five IL1RN TTG subjects compared to 5 TTG-0 controls. The sorted cellular suspensions are loaded on a 10× Genomics Chromium instrument to generate single-cell gel beads in emulsion (GEMs). Approximately 10,000 cells are loaded per channel. scRNAseq libraries are and run on an Illumina HiSeq 4000 as 2×150 paired-end reads for approximately >90% sequencing saturation. SEURAT v2.0 is used to analyze scRNAseq results within and across experimental conditions and provides a global view of changes in WBCs and allows for analysis of associated gene expression patterns in subgroups of cells. Candidate mechanisms are inferred from clinical, scRNAseq data, metabolomic data, and integration of results obtained by all 3 methods. iPathwayGuide is used to identify the dominant KEGG pathways associated with differentially expressed genes. The KEGG and Small Molecule Pathway Database (SMPDB) is used to predict differential metabolite changes. The integrated pathways are visualized with Metscape to infer causal mechanisms with emphasis on lipid genes. Data is integrated using generalized regression model (log-normal for counts data and linear regression model for continuous normal data) while adjusting for covariates such as age, sex, and baseline BMI. A log-normal mixed-effects model is then used to compare time-specific scRNAseq changes with changes in % weight loss between IL1RN TTG-½ vs. TTG-0 subjects following SG.

### C. Results

IL1RN risk haplotypes are observed to differentially regulate cellular pathways that promote inflammation and adipokine production. Subjects with the IL1RN TTG haplotype are observed to have higher levels of immune infiltration and proinflammatory immune cells compared to controls with the TTG-0 haplotype. The cellular site(s) of IL1RN expression are determined and ATM infiltration is observed to demonstrate greater inflammatory infiltrates in IL1RN TTG vs. TTG-0 carriers. WAT markers of inflammation, including assessment by scRNA-seq, are observed to decrease with bariatric surgery, with the extent to which activation of this pathway is reduced varying between haplotypes. The decreased expression of leptin is observed to be less in IL1RN TTG carriers.

V. The Associations of IL1RN Haplotypes with Appetite, Satiety, and Caloric Intake.

### A. Introduction

Leptin and ghrelin act at the level of the hypothalamus to impact feeding behaviors. Patients with the IL1RN TTG haplotype have higher levels of leptin pre- and post-bariatric surgery. This most likely reflects higher leptin resistance, which can lead to lower satiety and higher caloric intake. Validated measures of satiety, appetite, and dietary intake are used at multiple timepoints and a causal pathway analysis is conducted to elucidate the mechanisms of how the IL1RN TTG haplotype impacts weight loss and possibly weight regain in bariatric surgery patients. For all participants in the bariatric surgery cohort, appetite, satiety, and dietary intake is assessed at all available timepoints with the measures described below.

### B. Measures of Food Intake, Appetite and Satiety:

1. Dietary Measurement ASA 24*: To measure dietary energy intake at each time point, an automated, cost-effective and valid 24-hour dietary recall instrument is employed. To limit participant burden, participants perform one weekday measurement (self-administered in person with staff available to answer questions as needed) and one weekend measurement (self-administered at home).
2. Subjective Satiety and Hunger: For this assessment, participants are asked to fast at home for 8-hours prior to the day of testing. During the fasting period, participants are asked to abstain from any calorie containing foodstuff. On testing day, participants are asked to consume a mixed meal provided as 200 mL of Ensure Plus (Abbott Laboratories, North Chicago, Ill., USA) containing 300 kcal, 12.5 g protein, 40.4 g carbohydrate, 9.84 g fat and 154.86 g water. Subjective assessment of satiety and hunger is assessed before ingestion of Ensure and at 30 and 60 minutes from ingestion. Participants are instructed to mark the line corresponding to their satiety and hunger sensation at each particular time. Quantification of the measures is done by measuring the distance from the left end of the line to the mark.
3. Appetite and Eating behaviors: Appetite is assessed via a 35 item, self-reported Adult Eating Behaviour Questionnaire (AEBQ). The AEQB is adapted from the Children Eating Behaviour questionnaire and validated for use in adults by Hunot et al. Hunot et al. included patients with obesity in their validation and reported good internal reliability (α: 0.76-0.88) and re-test reliability (ICCs: 0.73-0.91). Eating behaviors are assessed using the revised and shortened Three-Factor Eating Questionnaire (TFEQ) which is an 18-item, self-administered questionnaire.

### C. Results

The ILRN TTG-½ haplotype is observed to be associated with decreased satiety, increased appetite and higher calorie intake, the mechanisms of which associations are observed to occur via differential changes in leptin and ghrelin.

Notwithstanding the appended claims, the disclosure is also defined by the following clauses:

1. A method for treating a subject for osteoarthritis, wherein the subject has been identified as having a TTG rs419598/rs315952/rs9005 haplotype, the method comprising:

administering to the subject a TTG indicated therapeutic regimen.

2. The method according to Clause 1, wherein the subject has a TTG-1 haplotype.
3. The method according to Clause 2, wherein the subject does not have a CTA haplotype.
4. The method according to Clause 1, wherein the subject has a TTG-2 haplotype.
5. The method according to any the preceding clauses, wherein the TTG indicated therapeutic regimen antagonizes interleukin-1 (IL-1) activity.
6. The method according to Clause 5, wherein the TTG indicated therapeutic regimen increases synovial fluid IL-1Ra concentration.
7. The method according to Clause 6, wherein the TTG indicated therapeutic regimen comprises intra-articularly administering a dosage to the subject comprising a nucleic acid coding sequence for a human interleukin-1 receptor antagonist (IL-1Ra).
8. The method according to Clause 7, wherein the coding sequence comprises a naturally occurring coding sequence.
9. The method according to Clause 7, wherein the coding sequence comprises a non-naturally occurring coding sequence.
10. The method according to Clause 9, wherein the coding sequence comprises a codon-optimized coding sequence.
11. The method according to any of Clauses 7 to 10, wherein the coding sequence is present in a vector.
12. The method according to Clause 10, wherein the vector is a viral vector.
13. The method according to Clause 12, wherein the viral vector is non-integrating viral vector.
14. The method according to Clause 13, wherein the non-integrating viral vector is an adeno-associated virus (AAV) vector.
15. The method according to Clause 14, wherein the AAV vector is selected from the group consisting of AAV1, AAV2, AAV2.5, AAV3, AAV4, AAV5, AAV6, AAV7, AAV8, AAV9, AAV10, AAV11, AAV12 and Anc80.
16. The method according to any of Clauses 7 to 15, wherein the coding sequence is operatively linked to a promoter.
17. The method according to Clause 16, wherein the promoter is a non-human promoter.
18. The method according to Clause 17, wherein the promoter is a viral promoter.
19. The method according to Clause 5, wherein the TTG indicated therapeutic regimen comprises administration of an IL-1 specific binding agent.
20. The method according to Clause 19, wherein the IL-1 specific binding agent specifically binds to IL-1a.
21. The method according to Clause 19, wherein the IL-1 specific binding agent specifically binds to IL-1β.
22. The method according to any of Clauses 19 to 21, wherein the IL-1 specific binding agent comprises an antibody or binding fragment thereof.
23. The method according to Clauses 19 to 21, wherein the IL-1 specific binding agent comprises an IL-1 soluble receptor (IL-1sR).
24. The method according to Clause 5, wherein the TTG indicated therapeutic regimen comprises administration of an IL-1 receptor specific binding agent.
25. The method according to Clause 24, wherein the IL-1 receptor specific binding agent comprises an antibody or binding fragment thereof.
26. The method according to Clause 5, wherein the TTG indicated therapeutic regimen comprises an IL-1 production inhibitor.
27. The method according to Clause 26, wherein the IL-1 production inhibitor comprises a small molecule.
28. The method according to Clause 27, wherein the small molecule comprises a caspase 1 inhibitor.
29. The method according to Clause 26, wherein the IL-1 production inhibitor comprises a nucleic acid IL-1 production inhibitor.
30. The method according to any of Clauses 1 to 4, wherein the TTG indicated therapeutic regimen comprises a disease modifying osteoarthritis drug (DMOAD) therapy.
31. The method according to Clause 30, wherein the DMOAD therapy is selected from the group consisting of: calcitonin, bisphosphonates, strontium ranelate, iNOS inhibitors, MMP-13 inhibitors, aggrecanse inhibitors, anti-IL 1β, doxycycline, cathepsin K inhibitors, BMP-7, TIMP-3, and FGF-18.
32. The method according to any of the preceding clauses, wherein the osteoarthritis is selected from the group consisting osteoarthritis of the hand, knee, hip, shoulder, ankle, elbow, temperomandibular joint, and spine, and combinations thereof.
33. The method according to Clause 32, wherein the osteoarthritis is osteoarthritis of the knee.
34. The method according to any of the preceding clauses, wherein the subject is a mammal.
35. The method according to Clause 34, wherein the mammal is a human.
36. The method according to Clause 35, wherein the human is an adult human.
37. The method according to any of Clauses 35 to 36, wherein human is non-Caucasian.
38. The method according to any of the preceding clauses, wherein the subject exhibits reduced plasma IL-1Ra as compared to a suitable control.
39. The method according to any of the preceding clauses, wherein the subject exhibits faster joint space narrowing as compared to a suitable control.
40. The method according to any of the preceding clauses, wherein the method further comprises haplotyping the subject.
41. The method according to Clause 40, wherein the haplotyping comprises assaying a blood sample from the subject.
42. The method according to Clause 41, wherein the blood sample comprises a peripheral blood sample.
43. The method according to Clause 42, wherein the peripheral blood sample comprises peripheral blood leukocytes (PBL).
44. A method for treating a subject for osteoarthritis, wherein the subject has been identified as either: (a) having a TTG rs419598/rs315952/rs9005 haplotype; or (b) not having a TTG rs419598/rs315952/rs9005 haplotype, the method comprising:

administering to the subject a TTG indicated therapeutic regimen that antagonizes interleukin-1 (IL-1) activity if the subject has a TTG rs419598/rs315952/rs9005 haplotype.

45. The method according to Clause 44, wherein the subject has a TTG-1 haplotype.
46. The method according to Clause 45, wherein the subject does not have a CTA haplotype.
47. The method according to Clause 44, wherein the subject has a TTG-2 haplotype.
48. The method according to any of Clauses 44 to 47, wherein the TTG indicated therapeutic regimen increases synovial fluid IL-1Ra concentration.
49. The method according to Clause 48, wherein the TTG indicated therapeutic regimen comprises intra-articularly administering a dosage to the subject comprising a nucleic acid coding sequence for a human interleukin-1 receptor antagonist (IL-1Ra).
50. The method according to Clause 49, wherein the coding sequence comprises a naturally occurring coding sequence.
51. The method according to Clause 49, wherein the coding sequence comprises a non-naturally occurring coding sequence.
52. The method according to Clause 451, wherein the coding sequence comprises a codon-optimized coding sequence.
53. The method according to any of Clauses 49 to 52, wherein the coding sequence is present in a vector.
54. The method according to Clause 53, wherein the vector is a viral vector.
55. The method according to Clause 54, wherein the viral vector is non-integrating viral vector.
56. The method according to Clause 55, wherein the non-integrating viral vector is an adeno-associated virus (AAV) vector.
57. The method according to Clause 56, wherein the AAV vector is selected from the group consisting of AAV1, AAV2, AAV2.5, AAV3, AAV4, AAV5, AAV6, AAV7, AAV8, AAV9, AAV10, AAV11, AAV12 and Anc80.
58. The method according to any of Clauses 49 to 57, wherein the coding sequence is operatively linked to a promoter.
59. The method according to Clause 58, wherein the promoter is a non-human promoter.
60. The method according to Clause 59, wherein the promoter is a viral promoter.
61. The method according to any of Clauses 44 to 47, wherein the TTG specific therapeutic regimen comprises administration of an IL-1 specific binding agent.
62. The method according to Clause 61, wherein the IL-1 specific binding agent specifically binds to IL-1α.
63. The method according to Clause 61, wherein the IL-1 specific binding agent specifically binds to IL-1β.
64. The method according to any of Clauses 61 to 63, wherein the IL-1 specific binding agent comprises an antibody or binding fragment thereof.
65. The method according to Clause 61, wherein the IL-1 specific binding agent comprises an IL-1 soluble receptor (IL-1sR).
66. The method according to any of Clauses 44 to 47, wherein the TTG indicated therapeutic regimen comprises administration of an IL-1 receptor specific binding agent.
67. The method according to Clause 666, wherein the IL-1 receptor specific binding agent comprises an antibody or binding fragment thereof.
68. The method according to any of Clauses 44 to 47, wherein the first therapeutic regimen comprises an IL-1 production inhibitor.
69. The method according to Clause 68, wherein the IL-1 production inhibitor comprises a small molecule.
70. The method according to Clause 69, wherein the small molecule comprises a caspase 1 inhibitor.
71. The method according to Clause 68, wherein the IL-1 production inhibitor comprises a nucleic acid IL-1 production inhibitor.
72. The method according to Clause 44, wherein the method comprises administering to the subject a second therapeutic regimen that is different from the TTG indicated therapeutic regimen if the subject does not have a TTG rs419598/rs315952/rs9005 haplotype
73. The method according to Clause 72, wherein the subject has a CTA haplotype.
74. The method according to Clause 73, wherein the subject has a CTA-1 haplotype.
75. The method according to Clause 73, wherein the subject has a CTA-2 haplotype.
76. The method according to any of Clauses 72 to 75, wherein the second therapeutic regimen comprises a conservative therapy.
77. The method according to Clause 76, wherein the conservative therapy is selected from the group consisting of activity modification, weight loss, physical therapy, steroid therapy, non-steroidal anti-inflammatory therapy, injection therapy, and combinations thereof.
78. The method according to any of Clauses 72 to 75 wherein the second therapeutic regimen comprises osteoarthritis therapy is selected from the group consisting of: acetaminophen therapy, non-steroidal anti-inflammatory drug therapy, opiate therapy, glucocorticoid therapy, hyaluronic acid therapy, stem cell therapy, autologous blood product therapy, and combinations thereof.
79. The method according to any of Clauses 44 to 78, wherein the osteoarthritis is selected from the group consisting osteoarthritis of the hand, knee, hip, shoulder, ankle, elbow, temperomandibular joint, and spine, and combinations thereof.
80. The method according to Clause 79, wherein the osteoarthritis is osteoarthritis of the knee.
81. The method according to any of Clauses 44 to 80, wherein the subject is a mammal.
82. The method according to Clause 81, wherein the mammal is a human.
83. The method according to Clause 82, wherein the human is an adult human.
84. The method according to any of Clauses 82 to 83, wherein human is non-Caucasian.
85. The method according to any of Clauses 44 to 84, wherein the subject exhibits reduced plasma IL-1Ra as compared to a suitable control.
86. The method according to any of Clauses 44 to 85, wherein the subject exhibits faster joint space narrowing as compared to a suitable control.
87. The method according to any of Clauses 44 to 86, wherein the method further comprises haplotyping the subject.
88. The method according to Clause 87, wherein the haplotyping comprises assaying a blood sample from the subject.
89. The method according to Clause 88, wherein the blood sample comprises a peripheral blood sample.
90. The method according to Clause 89, wherein the peripheral blood sample comprises peripheral blood leukocytes (PBL).
91. A method for treating a subject for osteoarthritis, wherein the subject has been identified as either: (a) having a TTG rs419598/rs315952/rs9005 haplotype; or (b) not having a TTG rs419598/rs315952/rs9005 haplotype, the method comprising:

administering to the subject a disease modifying osteoarthritis drug (DMOAD) therapy if the subject has a TTG rs419598/rs315952/rs9005 haplotype.

92. The method according to Clause 91, wherein the subject has a TTG-1 haplotype.
93. The method according to Clause 92, wherein the subject does not have a CTA haplotype.
94. The method according to Clause 91, wherein the subject has a TTG-2 haplotype.
95. The method according to any of Clauses 91 to 94, wherein the DMOAD therapy is selected from the group consisting of: calcitonin, bisphosphonates, strontium ranelate, iNOS inhibitors, MMP-13 inhibitors, aggrecanse inhibitors, anti-IL1β, doxycycline, cathepsin K inhibitors, BMP-7, TIMP-3, and FGF-18.
96. The method according to Clause 91, wherein the method comprises administering to the subject a second therapeutic regimen that is different from the DMOAD therapy if the subject does not have a TTG rs419598/lrs315952/rs9005 haplotype
97. The method according to Clause 96, wherein the subject has a CTA haplotype.
98. The method according to Clause 97, wherein the subject has a CTA-1 haplotype.
99. The method according to Clause 98, wherein the subject has a CTA-2 haplotype.
100. The method according to any of Clauses 96 to 99, wherein the second therapeutic regimen comprises a conservative therapy.
101. The method according to Clause 100, wherein the conservative therapy is selected from the group consisting of activity modification, weight loss, physical therapy, steroid therapy, non-steroidal anti-inflammatory therapy, injection therapy, and combinations thereof.
102. The method according to any of Clauses 96 to 00, wherein the second therapeutic regimen comprises an osteoarthritis therapy is selected from the group consisting of: acetaminophen therapy, non-steroidal anti-inflammatory drug therapy, opiate therapy, glucocorticoid therapy, hyaluronic acid therapy, stem cell therapy, autologous blood product therapy, and combinations thereof.
103. The method according to any of Clauses 91 to 102, wherein the osteoarthritis is selected from the group consisting osteoarthritis of the hand, knee, hip, shoulder, ankle, elbow, temperomandibular joint, and spine, and combinations thereof.
104. The method according to Clause 103, wherein the osteoarthritis is osteoarthritis of the knee.
105. The method according to any of Clauses 91 to 104, wherein the subject is a mammal.
106. The method according to Clause 105, wherein the mammal is a human.
107. The method according to Clause 106, wherein the human is an adult human.
108. The method according to any of Clauses 106 to 107, wherein human is non-Caucasian.
109. The method according to any of Clauses 91 to 108, wherein the subject exhibits reduced plasma IL-1Ra as compared to a suitable control.
110. The method according to any of Clauses 91 to 109, wherein the subject exhibits faster joint space narrowing as compared to a suitable control.
111. The method according to any of Clauses 91 to 110, wherein the method further comprises haplotyping the subject.
112. The method according to Clause 111, wherein the haplotyping comprises assaying a blood sample from the subject.
113. The method according to Clause 112, wherein the blood sample comprises a peripheral blood sample.
114. The method according to Clause 113, wherein the peripheral blood sample comprises peripheral blood leukocytes (PBL).
115. A method for treating a subject for osteoarthritis, wherein the subject reduced sIL-1Ra, the method comprising:

administering to the subject a TTG indicated therapeutic regimen.

116. The method according to Clause 115, wherein the TTG indicated therapeutic regimen antagonizes interleukin-1 (IL-1) activity.
117. The method according to Clause 116, wherein the TTG indicated therapeutic regimen increases synovial fluid IL-1Ra concentration.
118. The method according to Clause 117, wherein the TTG indicated therapeutic regimen comprises intra-articularly administering a dosage to the subject comprising a nucleic acid coding sequence for a human interleukin-1 receptor antagonist (IL-1Ra).
120. The method according to Clause 118, wherein the coding sequence comprises a naturally occurring coding sequence.
121. The method according to Clause 118, wherein the coding sequence comprises a non-naturally occurring coding sequence.
122. The method according to Clause 121, wherein the coding sequence comprises a codon-optimized coding sequence.
123. The method according to any of Clauses 118 to 122, wherein the coding sequence is present in a vector.
124. The method according to Clause 123, wherein the vector is a viral vector.
125. The method according to Clause 124, wherein the viral vector is non-integrating viral vector.
126. The method according to Clause 125, wherein the non-integrating viral vector is an adeno-associated virus (AAV) vector.
127. The method according to Clause 126, wherein the AAV vector is selected from the group consisting of AAV1, AAV2, AAV2.5, AAV3, AAV4, AAV5, AAV6, AAV7, AAV8, AAV9, AAV10, AAV11, AAV12 and Anc80.
128. The method according to any of Clauses 118 to 127, wherein the coding sequence is operatively linked to a promoter.
129. The method according to Clause 128, wherein the promoter is a non-human promoter.
130. The method according to Clause 129, wherein the promoter is a viral promoter.
131. The method according to Clause 116, wherein the TTG indicated therapeutic regimen comprises administration of an IL-1 specific binding agent.
132. The method according to Clause 131, wherein the IL-1 specific binding agent specifically binds to IL-1α.
133. The method according to Clause 131, wherein the IL-1 specific binding agent specifically binds to IL-1β.
134. The method according to any of Clauses 132 or 133, wherein the IL-1 specific binding agent comprises an antibody or binding fragment thereof.
135. The method according to Clause 131, wherein the IL-1 specific binding agent comprises an IL-1 soluble receptor (IL-1sR).
136. The method according to Clause 116, wherein the TTG indicated therapeutic regimen comprises administration of an IL-1 receptor specific binding agent.
137. The method according to Clause 136, wherein the IL-1 receptor specific binding agent comprises an antibody or binding fragment thereof.
138. The method according to Clause 116, wherein the TTG indicated therapeutic regimen comprises an IL-1 production inhibitor.
139. The method according to Clause 138, wherein the IL-1 production inhibitor comprises a small molecule.
140. The method according to Clause 139, wherein the small molecule comprises a caspase 1 inhibitor.
141. The method according to Clause 138, wherein the IL-1 production inhibitor comprises a nucleic acid IL-1 production inhibitor.
142. The method according to Clause 115, wherein the TTG indicated therapeutic regimen comprises a disease modifying osteoarthritis drug (DMOAD) therapy.
143. The method according to Clause 142, wherein the DMOAD therapy is selected from the group consisting of: calcitonin, bisphosphonates, strontium ranelate, iNOS inhibitors, MMP-13 inhibitors, aggrecanse inhibitors, anti-IL 1β, doxycycline, cathepsin K inhibitors, BMP-7, TIMP-3, and FGF-18.
144. The method according to any of Clauses 115 to 143, wherein the osteoarthritis is selected from the group consisting osteoarthritis of the hand, knee, hip, shoulder, ankle, elbow, temperomandibular joint, and spine, and combinations thereof.
145. The method according to Clause 144, wherein the osteoarthritis is osteoarthritis of the knee.
146. The method according to any of Clauses 115 to 145, wherein the subject is a mammal.
147. The method according to Clause 146, wherein the mammal is a human.
148. The method according to Clause 147, wherein the human is an adult human.
149. The method according to any of Clauses 147 to 148, wherein human is non-Caucasian.
150. The method according to any of Clauses 115 to 149, wherein the subject exhibits faster joint space narrowing as compared to a suitable control.
151. The method according to any of Clauses 115 to 150, wherein the method further comprises determining that the subject produces an inefficiently exported IL-1Ra.
152. The method according to clause 151, wherein the determining comprises assaying a sample from the subject for IL-1Ra.
153. The method according to Clause 152, wherein the sample comprises synovial fluid. 153. A method for treating a subject for osteoarthritis, wherein the subject is undergoing faster disease progression as compared to a suitable control, the method comprising: administering to the subject a TTG indicated therapeutic regimen.
154. The method according to Clause 153, wherein disease progression has been assessed using an imaging protocol.
155. The method according to Clause 154, wherein the imaging protocol comprises an X-ray protocol.
156. The method according to Clause 155, wherein the assessing comprises employing a scale.
157. The method according to Clause 156, wherein the scale comprises the Kellgren-Lawrence scale.
158. The method according to Clause 157, wherein the assessment results in a Kellgren-Lawrence score of 2, 3 or 4.
159. The method according to Clause 154, wherein the imaging protocol comprises a magnetic resonance imaging (MRI) protocol.
160. The method according to Clause 159, wherein the TTG indicated therapeutic regimen antagonizes interleukin-1 (IL-1) activity.
161. The method according to Clause 160, wherein the TTG indicated therapeutic regimen increases synovial fluid IL-1Ra concentration.
162. The method according to Clause 161, wherein the TTG indicated therapeutic regimen comprises intra-articularly administering a dosage to the subject comprising a nucleic acid coding sequence for a human interleukin-1 receptor antagonist (IL-1Ra).
163. The method according to Clause 162, wherein the coding sequence comprises a naturally occurring coding sequence.
164. The method according to Clause 162, wherein the coding sequence comprises a non-naturally occurring coding sequence.
165. The method according to Clause 164, wherein the coding sequence comprises a codon-optimized coding sequence.
166. The method according to any of Clauses 162 to 165, wherein the coding sequence is present in a vector.
167. The method according to Clause 166, wherein the vector is a viral vector.
168. The method according to Clause 167, wherein the viral vector is non-integrating viral vector.
169. The method according to Clause 168, wherein the non-integrating viral vector is an adeno-associated virus (AAV) vector.
170. The method according to Clause 169, wherein the AAV vector is selected from the group consisting of AAV1, AAV2, AAV2.5, AAV3, AAV4, AAV5, AAV6, AAV7, AAV8, AAV9, AAV10, AAV11, AAV12 and Anc80.
171. The method according to any of Clauses 162 to 170, wherein the coding sequence is operatively linked to a promoter.
172. The method according to Clause 171, wherein the promoter is a non-human promoter.
173. The method according to Clause 172, wherein the promoter is a viral promoter.
174. The method according to Clause 153, wherein the TTG indicated therapeutic regimen comprises administration of an IL-1 specific binding agent.
175. The method according to Clause 154, wherein the IL-1 specific binding agent specifically binds to IL-1α.
176. The method according to Clause 154, wherein the IL-1 specific binding agent specifically binds to IL-1β.
177. The method according to any of Clauses 175 to 176, wherein the IL-1 specific binding agent comprises an antibody or binding fragment thereof.
178. The method according to Clause 174, wherein the IL-1 specific binding agent comprises an IL-1 soluble receptor (IL-1sR).
179. The method according to Clause 153, wherein the TTG indicated therapeutic regimen comprises administration of an IL-1 receptor specific binding agent.
180. The method according to Clause 179, wherein the IL-1 receptor specific binding agent comprises an antibody or binding fragment thereof.
181. The method according to Clause 153, wherein the TTG indicated therapeutic regimen comprises an IL-1 production inhibitor.
182. The method according to Clause 181, wherein the IL-1 production inhibitor comprises a small molecule.
183. The method according to Clause 182, wherein the small molecule comprises a caspase 1 inhibitor.
184. The method according to Clause 181, wherein the IL-1 production inhibitor comprises a nucleic acid IL-1 production inhibitor.
185. The method according to Clause 153, wherein the TTG indicated therapeutic regimen comprises a disease modifying osteoarthritis drug (DMOAD) therapy.
186. The method according to Clause 185, wherein the DMOAD therapy is selected from the group consisting of: calcitonin, bisphosphonates, strontium ranelate, iNOS inhibitors, MMP-13 inhibitors, aggrecanse inhibitors, anti-IL1β, doxycycline, cathepsin K inhibitors, BMP-7, TIMP-3, and FGF-18.
187. The method according to any of Clauses 153 to 186, wherein the osteoarthritis is selected from the group consisting osteoarthritis of the hand, knee, hip, shoulder, ankle, elbow, temperomandibular joint, and spine, and combinations thereof.
188. The method according to Clause 187, wherein the osteoarthritis is osteoarthritis of the knee.
189. The method according to any of Clauses 153 to 188, wherein the subject is a mammal.
190. The method according to Clause 189, wherein the mammal is a human.
191. The method according to Clause 190, wherein the human is an adult human.
192. The method according to any of Clauses 190 to 191, wherein human is non-Caucasian.
193. The method according to any of Clauses 153 to 192, wherein the subject exhibits reduced plasma IL-1Ra as compared to a suitable control.
194. The method according to any of Clauses 153 to 193, wherein the method further comprises assessing disease progression using an imaging protocol.
195. The method according to Clause 194, wherein the imaging protocol comprises an X-ray protocol.
196. The method according to Clause 195, wherein the assessing comprises employing a scale.
197. The method according to Clause 196, wherein the scale comprises the Kellgren-Lawrence scale.
198. The method according to Clause 197, wherein the assessment results in a Kellgren-Lawrence score of 2, 3 or 4.
200. The method according to Clause 194, wherein the imaging protocol comprises a magnetic resonance imaging (MRI) protocol.
201. A method for treating a subject for arthritis, wherein the subject has been identified as having a TTG rs419598/rs315952/rs9005 haplotype, the method comprising:

administering to the subject a TTG indicated therapeutic regimen.

202. The method according to Clause 201, wherein the subject has a TTG-1 haplotype.
203. The method according to Clause 202, wherein the subject does not have a CTA haplotype.
204. The method according to Clause 201, wherein the subject has a TTG-2 haplotype.
205. The method according to any Clauses 201 to 204, wherein the TTG indicated therapeutic regimen antagonizes interleukin-1 (IL-1) activity.
206. The method according to Clause 205, wherein the TTG indicated therapeutic regimen increases synovial fluid IL-1Ra concentration.
207. The method according to Clause 206, wherein the TTG indicated therapeutic regimen comprises intra-articularly administering a dosage to the subject comprising a nucleic acid coding sequence for a human interleukin-1 receptor antagonist (IL-1Ra).
208. The method according to Clause 207, wherein the coding sequence comprises a naturally occurring coding sequence.
209. The method according to Clause 207, wherein the coding sequence comprises a non-naturally occurring coding sequence.
210. The method according to Clause 209, wherein the coding sequence comprises a codon-optimized coding sequence.
211. The method according to any of Clauses 207 to 210, wherein the coding sequence is present in a vector.
212. The method according to Clause 210, wherein the vector is a viral vector.
213. The method according to Clause 212, wherein the viral vector is non-integrating viral vector.
214. The method according to Clause 213, wherein the non-integrating viral vector is an adeno-associated virus (AAV) vector.
215. The method according to Clause 214, wherein the AAV vector is selected from the group consisting of AAV1, AAV2, AAV2.5, AAV3, AAV4, AAV5, AAV6, AAV7, AAV8, AAV9, AAV10, AAV11, AAV12 and Anc80.
216. The method according to any of Clauses 207 to 215, wherein the coding sequence is operatively linked to a promoter.
217. The method according to Clause 216, wherein the promoter is a non-human promoter.
218. The method according to Clause 217, wherein the promoter is a viral promoter.
219. The method according to Clause 215, wherein the TTG indicated therapeutic regimen comprises administration of an IL-1 specific binding agent.
220. The method according to Clause 219, wherein the IL-1 specific binding agent specifically binds to IL-1a.
221. The method according to Clause 219, wherein the IL-1 specific binding agent specifically binds to IL-1β.
222. The method according to any of Clauses 219 to 221, wherein the IL-1 specific binding agent comprises an antibody or binding fragment thereof.
223. The method according to Clauses 219 to 221, wherein the IL-1 specific binding agent comprises an IL-1 soluble receptor (IL-1sR).
224. The method according to Clause 215, wherein the TTG indicated therapeutic regimen comprises administration of an IL-1 receptor specific binding agent.
225. The method according to Clause 224, wherein the IL-1 receptor specific binding agent comprises an antibody or binding fragment thereof.
226. The method according to Clause 205, wherein the TTG indicated therapeutic regimen comprises an IL-1 production inhibitor.
227. The method according to Clause 226, wherein the IL-1 production inhibitor comprises a small molecule.
228. The method according to Clause 227, wherein the small molecule comprises a caspase 1 inhibitor.
229. The method according to Clause 226, wherein the IL-1 production inhibitor comprises a nucleic acid IL-1 production inhibitor.
230. The method according to any of Clauses 201 to 229, wherein the arthritis is osteoarthritis.
231. The method according to Clause 230, wherein the TTG indicated therapeutic regimen comprises a disease modifying osteoarthritis drug (DMOAD) therapy.
232. The method according to Clause 231, wherein the DMOAD therapy is selected from the group consisting of: calcitonin, bisphosphonates, strontium ranelate, iNOS inhibitors, MMP-13 inhibitors, aggrecanse inhibitors, anti-IL1β, doxycycline, cathepsin K inhibitors, BMP-7, TIMP-3, and FGF-18.
233. The method according to any of Clauses 230 to 232, wherein the osteoarthritis is selected from the group consisting osteoarthritis of the hand, knee, hip, shoulder, ankle, elbow, temperomandibular joint, and spine, and combinations thereof.
234. The method according to Clause 233, wherein the osteoarthritis is osteoarthritis of the knee.
235. The method according to any of Clause 201 to 229, wherein the arthritis is rheumatoid arthritis.
236. The method accordin to Clause 235, wherein the TTG indicated therapeutic regimen comprises a disease-modifying antirheumatic drug (DMARD) therapy.
237. The method according to Clause 236, wherein the DMARD therapy comprises TNF-alpha inhibitor therapy.
238. The method according to any of Clauses 201 to 237, wherein the subject is a mammal.
239. The method according to Clause 238, wherein the mammal is a human.
240. The method according to Clause 239, wherein the human is an adult human.
241. The method according to any of Clauses 239 to 240, wherein human is non-Caucasian.
242. The method according to any of Clauses 201 to 241, wherein the method further comprises haplotyping the subject.
243. The method according to Clause 242, wherein the haplotyping comprises assaying a blood sample from the subject.
244. The method according to Clause 243, wherein the blood sample comprises a peripheral blood sample.
245. The method according to Clause 244, wherein the peripheral blood sample comprises peripheral blood leukocytes (PBL).
246. A method for treating a subject for rheumatoid arthritis, wherein the subject has been identified as either: (a) having a TTG rs419598/rs315952/rs9005 haplotype; or (b) not having a TTG rs419598/rs315952/rs9005 haplotype, the method comprising:

administering to the subject a TTG indicated therapeutic regimen if the subject has a TTG rs419598/rs315952/rs9005 haplotype.

247. The method according to Clause 246, wherein the subject has a TTG-1 haplotype.
248. The method according to Clause 247, wherein the subject does not have a CTA haplotype.
249. The method according to Clause 247, wherein the subject has a TTG-2 haplotype.
250. The method according to any of Clauses 246 to 249, wherein the TTG indicated therapeutic regimen comprises DMARD therapy.
251. The method according to Clause 250, wherein the DMARD therapy comprises IL-1 inhibitor therapy.
252. The method according to Clause 250, wherein the DMARD therapy comprises TNF-alpha inhibitor therapy.
253. The method according to Clause 246, wherein the method comprises administering to the subject a second therapeutic regimen that is different from the TTG indicated therapeutic regimen if the subject does not have a TTG rs419598/rs315952/rs9005 haplotype
254. The method according to Clause 253, wherein the subject has a CTA haplotype.
255. The method according to Clause 254, wherein the subject has a CTA-1 haplotype.
256. The method according to Clause 254, wherein the subject has a CTA-2 haplotype.
257. The method according to any of Clauses 253 to 256, wherein the second therapeutic regimen comprises a conservative therapy.
258. The method according to any of Clauses 246 to 257, wherein the subject is a mammal.
259. The method according to Clause 258, wherein the mammal is a human.
260. The method according to Clause 259, wherein the human is an adult human.
261. The method according to any of Clauses 259 to 260, wherein human is non-Caucasian.
262. The method according to any of Clauses 246 to 261, wherein the method further comprises haplotyping the subject.
263. The method according to Clause 262, wherein the haplotyping comprises assaying a blood sample from the subject.
264. The method according to Clause 263, wherein the blood sample comprises a peripheral blood sample.
265. The method according to Clause 264, wherein the peripheral blood sample comprises peripheral blood leukocytes (PBL).
266. A method comprising determining a bariatric surgery patient's rs419598/rs315952/rs9005 haplotype.
267. The method according to Clause 266, wherein when the bariatric surgery patient has a TTG rs419598/rs315952/rs9005 haplotype, the method comprises predicting reduced weight loss relative to a bariatric surgery patient not having a TTG rs419598/rs315952/rs9005 haplotype.
268. The method according to Clauses 266 or 267, wherein when the bariatric surgery patient has a TTG rs419598/rs315952/rs9005 haplotype, the method comprises predicting increased obesity-associated inflammation relative to a bariatric surgery patient not having a TTG rs419598/rs315952/rs9005 haplotype.
269. The method according to any of Clauses 266 to 268, wherein the bariatric surgery patient is a patient that will or has undergone a bariatric surgery selected from the group consisting of: gastric bypass, sleeve gastrectomy, adjustable gastric band, and biliopancreatic diversion with duodenal switch.
270. The method according to Clause 269, wherein the bariatric surgery is sleeve gastrectomy.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method for treating a subject for osteoarthritis, wherein the subject has been identified as having a TTG rs419598/rs315952/rs9005 haplotype, the method comprising:
administering to the subject a TTG indicated therapeutic regimen.

2. The method according to claim 1, wherein the subject has a TTG-1 haplotype.

3. The method according to claim 2, wherein the subject does not have a CTA haplotype.

4. The method according to claim 1, wherein the subject has a TTG-2 haplotype.

5. The method according to claim 1, wherein the TTG indicated therapeutic regimen antagonizes interleukin-1 (IL-1) activity.

6. The method according to claim 5, wherein the TTG indicated therapeutic regimen increases synovial fluid IL-1Ra concentration.

7. The method according to claim 6, wherein the TTG indicated therapeutic regimen comprises intra-articularly administering a dosage to the subject comprising a nucleic acid coding sequence for a human interleukin-1 receptor antagonist (IL-1Ra).

8. The method according to claim 7, wherein the coding sequence comprises a naturally occurring coding sequence.

9. The method according to claim 7, wherein the coding sequence comprises a non-naturally occurring coding sequence.

10. The method according to claim 9, wherein the coding sequence comprises a codon-optimized coding sequence.

11. The method according to claim 7, wherein the coding sequence is present in a vector.

12. The method according to claim 11, wherein the vector is a viral vector.

13. The method according to claim 12, wherein the viral vector is non-integrating viral vector.

14. The method according to claim 1, wherein the TTG indicated therapeutic regimen comprises a disease modifying osteoarthritis drug (DMOAD) therapy.

15. The method according to claim 1, wherein the method further comprises haplotyping the subject.
